SEQUENCE LISTING (154 sequences)

SEQ ID NO: 1 - RNA, 35 nt, Clostridium sporogenes
aucuuuccuccacuaggucggcguccaagaggaug

SEQ ID NO: 2 - RNA, 11 nt, Clostridium sporogenes
aaaggaggugu

SEQ ID NO: 3 - RNA, 60 nt, Artificial Sequence
  riboswitch
ggugauaccagcaucgucuugaugcccuuggcagcacccugcuaagguaacaacaagaug

SEQ ID NO: 4 - RNA, 63 nt, Artificial Sequence
  riboswitch
ggugauaccagcaucgucuugaugcccuuggcagcacccugcuaaggagguaacaacaagaug

SEQ ID NO: 5 - RNA, 63 nt, Artificial Sequence; identical to SEQ ID NO: 4
  riboswitch

SEQ ID NO: 6 - RNA, 60 nt, Artificial Sequence
  riboswitch
ggugauaccagcaucgucuugaugcccuuggcagcacccugcuaaggagguaacaacaug

SEQ ID NO: 7 - RNA, 60 nt, Artificial Sequence
  riboswitch
ggugauaccagcaucgucuugaugcccuuggcagcacccugcuaaggagguaacuuaaug

SEQ ID NO: 8 - RNA, 60 nt, Artificial Sequence
  riboswitch
ggugauaccagcaucgucuugaugcccuuggcagcacccugcuaaggagguguguuaaug

SEQ ID NO: 9 - RNA, 61 nt, Artificial Sequence
  riboswitch
ggugauaccagcaucgucuugaugcccuuggcagcacccugcuaaggaggucaacaagaug

SEQ ID NO: 10 - DNA, 841 bp, Bacillus megaterium
atgcaaatattgtggaaaaagtatgttaaagaaaactttgaaatgaatgtagatgaatgtggtatagaacaaggtataccaggattaggatataactatgaagtattgaaaaatgctgttattcattacgtaactaagggatatggaacttttaaatttaatggtaaggtatataacttaaaacaaggtgatatttttatactactaaaaggtatgcaagttgagtatgtggcttctattgatgatccttgggaatactactggataggatttagtggttcaaatgctaatgagtatttaaatagaacttctattactaactcctgtgttgctaattgtgaagaaaactcaaaaattccacagataatattaaatatgtgcgaaatatcaaaaacttataatccttcaagatctgatgacatactattactaaaagaactttactcattattgtacgcacttatagaagaattcccaaaaccttttgaatacaaagataaggaattacacacatatattcaagatgctcttaatttcattaattctaattacatgcatagcataactgttcaagaaattgctgattatgtgaacttaagtagaagttatttatataaaatgttcataaaaaaccttggaatttctcctcaaagatatttaataaaccttagaatgtacaaagccacccttttattaaaaagcactaaacttcctataggagaagtcgcaagtagtgtaggttatagtgactccctgttattttcaaaaactttttcaaaacatttttcaatgtctccactaaattacagaaataatcaagtaaataaaccaagtatataaa

SEQ ID NO: 11 - DNA, 30 bp, Bacillus megaterium
taagaaacattataacatattttagaactt

SEQ ID NO: 12 - DNA, 822 bp, Bacillus subtilis
atggaagaaacaaagatcaatgttatcattgcagatgataataaggaattttgtaacatattaagtgattatttgttaaatcaaagagacataatggttacaggtatagctaatgatggagtagaagctcttaaattggtggaagaaaaaaaaccagatttaataatactagatataataatgcctcatttagatggattaggagttttagaaaaattaaatagtatagatattactcctatgccaagggtaattgttttgtctgctgtaggacaggataagattactcaaagggctataaacttaggtgcagattattatgtagttaaaccttttgatatggacgtttttaccaagagaataagacaaatgtttaacaataccataagtagcgaagaagttagaagaccagtagtaatgccagaaacatgttcacaagaaaaaatatcattatccaaaaatgaacctgcagatttagaacaagaaataacagatattatacatgaaataggtgtaccagctcatataaaaggatatatgtatttgagagaagctataactatggtagttaatgacatggaacttttatccgcagtgacaaaggagttgtacccttcaatagctaaaaaatataatactacagcaagtagagtagaaagagctataagacatgctatagaagtagcttggtcaagaggtcaagttgaaactataaacaatatatttgggtatactataaataatggaaagggaaaaccaacaaattctgagtttattgcaatggtagctgataaattaagactaaaaaatagagtttgctaa

SEQ ID NO: 13 - DNA, 20 bp, Bacillus megaterium
agaaacattataacatattt

SEQ ID NO: 14 - DNA, 28 bp, Artificial Sequence
  primer
tataggtctcaatggtatttgaaaaaat

SEQ ID NO: 15 - DNA, 31 bp, Artificial Sequence
  primer
tatactcgagttaactatttatcaattcctg

SEQ ID NO: 16 - DNA, 36 bp, Artificial Sequence
  primer
cagcggccgcatggtatttgaaaaaattgataaaaa

SEQ ID NO: 17 - DNA, 31 bp, Artificial Sequence
  primer
tatagcggccgcaaatattcttatatttaga

SEQ ID NO: 18 - DNA, 27 bp, Artificial Sequence
  primer
tataggtctcaccatttctacctccta

SEQ ID NO: 19 - DNA, 60 bp, Artificial Sequence
  primer
aaagcagtttatttaaaataaattcttctaaatataagaatattttaaagaaatatcttt

SEQ ID NO: 20 - DNA, 51 bp, Artificial Sequence
  primer
ttataagattatatagaactttttaactattctaaaagattaatttacata

SEQ ID NO: 21 - DNA, 51 bp, Artificial Sequence; identical to SEQ ID NO: 20
  primer

SEQ ID NO: 22 - DNA, 49 bp, Artificial Sequence
  primer
aaaagttctatataatcttataaattttaataactaatatataaagata

SEQ ID NO: 23 - DNA, 40 bp, Artificial Sequence
  primer
cacagcggccgcttctacctcctaacctataaaattagcc

SEQ ID NO: 24 - DNA, 50 bp, Artificial Sequence
  primer
cacacatatgaccctcccaatacatttaaaataattatgtattcatgaaa

SEQ ID NO: 25 - DNA, 60 bp, Artificial Sequence
  primer
ggccggtctcttaataactaatatataaagatatttctttaaaatattcttatatttaga

SEQ ID NO: 26 - DNA, 58 bp, Artificial Sequence
  primer
ccgcggccgctaatttagatattaattctaaattaagtgaaattaatatagtaattat

SEQ ID NO: 27 - DNA, 54 bp, Artificial Sequence
  primer
ggccggccggtctctattatataagattataagaaacattataacatattttag

SEQ ID NO: 28 - DNA, 54 bp, Artificial Sequence
  primer
ggccggccggtctctattagattataagaaacattataacatattttagaactt

SEQ ID NO: 29 - DNA, 54 bp, Artificial Sequence
  primer
ggccggccggtctctattataagaaacattataacatattttagaactttttaa

SEQ ID NO: 30 - DNA, 54 bp, Artificial Sequence
  primer
ggccggccggtctctattaaacattataacatattttagaactttttaactatt

SEQ ID NO: 31 - DNA, 54 bp, Artificial Sequence
  primer
ggccggccggtctctattatataacatattttagaactttttaactattctaaa

SEQ ID NO: 32 - DNA, 55 bp, Artificial Sequence
  primer
ggccggccggtctctattaacatattttagaactttttaactattctaaaagatt

SEQ ID NO: 33 - DNA, 55 bp, Artificial Sequence
  primer
ggccggccggtctctattattttagaactttttaactattctaaaagattaattt

SEQ ID NO: 34 - DNA, 55 bp, Artificial Sequence
  primer
ggccggccggtctctattagaactttttaactattctaaaagattaatttacata

SEQ ID NO: 35 - DNA, 55 bp, Artificial Sequence
  primer
ggccggccggtctctattattttaactattctaaaagattaatttacatattaac

SEQ ID NO: 36 - DNA, 55 bp, Artificial Sequence
  primer
ggccggccggtctctattaactattctaaaagattaatttacatattaacattta

SEQ ID NO: 37 - DNA, 55 bp, Artificial Sequence
  primer
ggccggccggtctctattatctaaaagattaatttacatattaacatttaattat

SEQ ID NO: 38 - DNA, 62 bp, Artificial Sequence
  primer
ggccggccggtctctattatttaatataagattataagaaacattataacatattttagaac

SEQ ID NO: 39 - DNA, 68 bp, Artificial Sequence
  primer
ggccggccggtctctattaaaattatattgattataagaaacattataacatattttagaacttttta

SEQ ID NO: 40 - DNA, 74 bp, Artificial Sequence
  primer
ggccggccggtctctattaaaatttataactaattaagaaacattataacatattttagaactttttaactatt

SEQ ID NO: 41 - DNA, 74 bp, Artificial Sequence
  primer
ggccggccggtctctattaaaatttataagattaattctaacattataacatattttagaactttttaactatt

SEQ ID NO: 42 - DNA, 74 bp, Artificial Sequence
  primer
ggccggccggtctctattaaaatttataagattataagattgtatataacatattttagaactttttaactatt

SEQ ID NO: 43 - DNA, 74 bp, Artificial Sequence
  primer
ggccggccggtctctattaaaatttataagattataagaaacatatattcatattttagaactttttaactatt

SEQ ID NO: 44 - DNA, 74 bp, Artificial Sequence
  primer
ggccggccggtctctattaaaatttataagattataagaaacattataagtatatttagaactttttaactatt

SEQ ID NO: 45 - DNA, 81 bp, Artificial Sequence
  primer
ggccggccggtctctattaaaatttataagattataagaaacattataacatataaatcaactttttaactattctaaaag

SEQ ID NO: 46 - DNA, 88 bp, Artificial Sequence
  primer
ggccggccggtctctattaaaatttataagattataagaaacattataacatattttagttgaatttaactattctaaaagattaatt

SEQ ID NO: 47 - DNA, 92 bp, Artificial Sequence
  primer
ggccggccggtctctattaaaatttataagattataagaaacattataacatattttagaacttaaattctattctaaaagattaatttaca

SEQ ID NO: 48 - DNA, 92 bp, Artificial Sequence
  primer
ggccggccggtctctattaaaatttataagattataagaaacattataacatattttagaactttttaagataactaaaagattaatttaca

SEQ ID NO: 49 - DNA, 37 bp, Artificial Sequence
  primer
ttatatacttggtttatttacttgattatttctgtaa

SEQ ID NO: 50 - DNA, 23 bp, Artificial Sequence
  primer
tattgtatagcttggtatcatct

SEQ ID NO: 51 - DNA, 37 bp, Artificial Sequence
  primer
atatgcggccgcgtgtagtagcctgtgaaataagtaa

SEQ ID NO: 52 - DNA, 28 bp, Artificial Sequence
  primer
tataggtctcaatggtatttgaaaaaat

SEQ ID NO: 53 - DNA, 28 bp, Artificial Sequence
  primer
tataggtctcaatggtatttgaaaaaat

SEQ ID NO: 54 - DNA, 31 bp, Artificial Sequence; identical to SEQ ID NO: 15
  primer

SEQ ID NO: 55 - DNA, 36 bp, Artificial Sequence; identical to SEQ ID NO: 16
  primer

SEQ ID NO: 56 - DNA, 40 bp, Artificial Sequence
  primer
ggcggtaccgatttatattttaccatgtttttattaattt

SEQ ID NO: 57 - DNA, 61 bp, Artificial Sequence
  primer
cccggtaccaatacgactcactataggttccggtgataccagcatcgtcttgatgcccttg

SEQ ID NO: 58 - DNA, 61 bp, Artificial Sequence
  primer
ccggtctcaccatgttgttgcctccttagcagggtgctgccaagggcatcaagacgatgct

SEQ ID NO: 59 - DNA, 61 bp, Artificial Sequence; identical to SEQ ID NO: 58
  primer

SEQ ID NO: 60 - DNA, 63 bp, Artificial Sequence
  primer
ccggtctcaccatcttgttgttacctccttagcagggtgctgccaagggcatcaagacgatgc

SEQ ID NO: 61 - DNA, 59 bp, Artificial Sequence
  primer
ccggtctcaccatgttgttacctccttagcagggtgctgccaagggcatcaagacgatg

SEQ ID NO: 62 - DNA, 60 bp, Artificial Sequence
  primer
ccggtctcaccattaagttacctccttagcagggtgctgccaagggcatcaagacgatgc

SEQ ID NO: 63 - DNA, 60 bp, Artificial Sequence
  primer
ccggtctcaccattaacacacctccttagcagggtgctgccaagggcatcaagacgatgc

SEQ ID NO: 64 - DNA, 61 bp, Artificial Sequence
  primer
ccggtctcaccatcttgttgacctccttagcagggtgctgccaagggcatcaagacgatgc

SEQ ID NO: 65 - DNA, 63 bp, Artificial Sequence
  primer
ccggtctcaccattggtcgtacactcccttagcagggtgctgccaagggcatcaagacgatgc

SEQ ID NO: 66 - DNA, 42 bp, Artificial Sequence
  primer
cccggtctctattgaaaaattacacaactttatacgatttat

SEQ ID NO: 67 - DNA, 54 bp, Artificial Sequence
  primer
ggtctcacaatacgactcactataggtgataccagcatcgtcttgatgcccttg

SEQ ID NO: 68 - DNA, 59 bp, Artificial Sequence
  primer
aaacttaacttcatgttaaaaacttgttaaaatataaatcgtataaagttgtgtaattt

SEQ ID NO: 69 - DNA, 60 bp, Artificial Sequence
  primer
atttatattttaacaagtttttaacatgaagttaagttttttttatataactgtataacc

SEQ ID NO: 70 - DNA, 23 bp, Artificial Sequence
  primer
aaatgtgaaatccgtcacatact

SEQ ID NO: 71 - DNA, 37 bp, Artificial Sequence
  primer
ggtatcatctcatcatatatccccaattcaccatctt

SEQ ID NO: 72 - DNA, 38 bp, Artificial Sequence
  primer
tataggcgcgcctttacaatcattgcaatataataaag

SEQ ID NO: 73 - DNA, 18 bp, Artificial Sequence
  primer
tgtaaaacgacggccagt

SEQ ID NO: 74 - DNA, 18 bp, Artificial Sequence
  primer
caggaaacagctatgacc

SEQ ID NO: 75 - DNA, 41 bp, Artificial Sequence
  primer
ccggtctcaatgcaaatattgtggaaaaagtatgttaaaga

SEQ ID NO: 76 - DNA, 63 bp, Artificial Sequence
  primer
ccggtctctgcatcttgttgttacctccttagcagggtgctgccaagggcatcaagacgatgc

SEQ ID NO: 77 - DNA, 60 bp, Artificial Sequence
  primer
ccggtctctgcattaagttacctccttagcagggtgctgccaagggcatcaagacgatgc

SEQ ID NO: 78 - DNA, 60 bp, Artificial Sequence
  primer
ccggtctctgcattaacacacctccttagcagggtgctgccaagggcatcaagacgatgc

SEQ ID NO: 79 - DNA, 39 bp, Artificial Sequence
  primer
ggcggtaccgagctttgcaaagcagtttatcaacataaa

SEQ ID NO: 80 - DNA, 39 bp, Artificial Sequence
  primer
ggcggtacctataaaattagccaattttatagtagtctt

SEQ ID NO: 81 - DNA, 40 bp, Artificial Sequence
  primer
gccccctgcaggagtaatataaatgttatagatatattaa

SEQ ID NO: 82 - DNA, 46 bp, Artificial Sequence
  primer
ccggtctcaaaccttatttttgttctctactacctggttttacaaa

SEQ ID NO: 83 - DNA, 120 bp, Artificial Sequence
  primer
cccgggtctcaggtttcccgactggaaagcgggcagtgaccaggcatcaaataaaacgaaaggctcagtcgaaagactgggcctttcgttttatctgttgtttgtcggtgaacgctctct

SEQ ID NO: 84 - DNA, 116 bp, Artificial Sequence
  primer
cccgcggccgcagttaattattgattataaacgcagaaaggcccacccgaaggtgagccagtgtgactctagtagagagcgttcaccgacaaacaacagataaaacgaaaggccca

SEQ ID NO: 85 - DNA, 55 bp, Artificial Sequence
  primer
ggccggtctcaattgatacatttaaaataattatgtattcatgaaacatgattgt

SEQ ID NO: 86 - DNA, 37 bp, Artificial Sequence; identical to SEQ ID NO: 49
  primer

SEQ ID NO: 87 - DNA, 23 bp, Artificial Sequence
  primer
tattgtatagcttggtatcatct

SEQ ID NO: 88 - DNA, 33 bp, Artificial Sequence
  primer
gaagacttagaaaattatatgaaagaagaaggt

SEQ ID NO: 89 - DNA, 22 bp, Artificial Sequence
  primer
caaaagcacttgaaaatgcatg

SEQ ID NO: 90 - DNA, 110 bp, Artificial Sequence
  sequence result
aaaataaaaagcttttatgttgataaactgctttgcaaagctctcatgtaaatgtttaatataagactactataaaattggctaattttataggttaggaggtagaaatg

SEQ ID NO: 91 - DNA, 149 bp, Artificial Sequence
  sequence result
taactattctaaaagattaatttacatattaacataattatgggtaaaaactattttgaaaaatgatttatatggaattatgtttcttaaatatacaatcatgtttcatgaatacataattattttaaatgtattgggagggtcatatg

SEQ ID NO: 92 - DNA, 32 bp, Artificial Sequence
  adaptor
tttttttctcatgtaaatgtttaatataagac

SEQ ID NO: 93 - DNA, 27 bp, Artificial Sequence
  adaptor
tttttttgggtaaaaactattttgaag

SEQ ID NO: 94 - DNA, 40 bp, Artificial Sequence
  motif
aatcctcctaggaaacattataacatgttttaaatacaaa

SEQ ID NO: 95 - DNA, 40 bp, Artificial Sequence; identical to SEQ ID NO: 94
  motif

SEQ ID NO: 96 - DNA, 40 bp, Artificial Sequence; identical to SEQ ID NO: 94
  motif

SEQ ID NO: 97 - DNA, 40 bp, Artificial Sequence
  motif
tatatattttggaaacattataacatatttgagcactata

SEQ ID NO: 98 - DNA, 40 bp, Artificial Sequence
  motif
ccgcaagcaaggtaacatagcgaccaattttcttcatgat

SEQ ID NO: 99 - DNA, 40 bp, Artificial Sequence; identical to SEQ ID NO: 98
  motif

SEQ ID NO: 100 - DNA, 40 bp, Artificial Sequence
  motif
taagattataagaaacattataacatattttagaactttt

SEQ ID NO: 101 - DNA, 40 bp, Artificial Sequence; identical to SEQ ID NO: 100
  motif

SEQ ID NO: 102 - DNA, 40 bp, Artificial Sequence
  motif
ctttaaatatggaaacattttaacatatacagtgacgatt

SEQ ID NO: 103 - DNA, 40 bp, Artificial Sequence
  motif
ccgcaagcaaggtaacatagcgaccaaatttcttcatggt

SEQ ID NO: 104 - DNA, 40 bp, Artificial Sequence
  motif
taatatgaaatgaaacattttgccatatatcatcaccatt

SEQ ID NO: 105 - DNA, 40 bp, Artificial Sequence
  motif
tcttattgtacaaaatgaaaacgctttatttaatggtctt

SEQ ID NO: 106 - DNA, 40 bp, Artificial Sequence; identical to SEQ ID NO: 105
  motif

SEQ ID NO: 107 - DNA, 40 bp, Artificial Sequence; identical to SEQ ID NO: 105
  motif

SEQ ID NO: 108 - DNA, 40 bp, Artificial Sequence
  motif
taaaaacaacaaacatgaaagcgctttacttaaaaaggat

SEQ ID NO: 109 - DNA, 40 bp, Artificial Sequence
  motif
actataactataacatgtaagcgctttataataaaatagg

SEQ ID NO: 110 - DNA, 40 bp, Artificial Sequence
  motif
actataactataacatgtaagcgctttataatcaaatagg

SEQ ID NO: 111 - DNA, 40 bp, Artificial Sequence; identical to SEQ ID NO: 110
  motif

SEQ ID NO: 112 - DNA, 40 bp, Artificial Sequence
  motif
agcagtttatcaacataaaagctttttattttaaaataaa

SEQ ID NO: 113 - DNA, 40 bp, Artificial Sequence; identical to SEQ ID NO: 112
  motif

SEQ ID NO: 114 - DNA, 40 bp, Artificial Sequence
  motif
tatcaattaacgaaaagaacgcgttatattccgtacatga

SEQ ID NO: 115 - DNA, 40 bp, Artificial Sequence
  motif
aaaatgattacaaaatgcaggggaattatgttaataaatt

SEQ ID NO: 116 - DNA, 40 bp, Artificial Sequence
  motif
acattataaatgaaatgaaaaggattaattttactttaaa

SEQ ID NO: 117 - DNA, 40 bp, Artificial Sequence
  motif
aagcttcaaggcaaaaggaggggcttcattgtctttatta

SEQ ID NO: 118 - DNA, 40 bp, Artificial Sequence
  motif
actttttaagtgaaacaaaagagttttattatctatctcg

SEQ ID NO: 119 - DNA, 40 bp, Artificial Sequence
  motif
tgctttcagtataaatgaaagcgttaaataatgtaatgca

SEQ ID NO: 120 - DNA, 40 bp, Artificial Sequence
  moif
tattccatttaaaagtgtaaaggaatcactaaaaaatatt

SEQ ID NO: 121 - DNA, 20 bp, Bacillus megaterium
caacataaaagctttttatt

SEQ ID NO: 122 - DNA, 90 bp, Artificial Sequence
  plasmid
aaatttataagattataagaaacattataacatattttagaactttttaactattctaaaagattaatttacatattaacatttaattat

SEQ ID NO: 123 - DNA, 85 bp, Artificial Sequence
  plasmid
tataagattataagaaacattataacatattttagaactttttaactattctaaaagattaatttacatattaacatttaattat

SEQ ID NO: 124 - DNA, 80 bp, Artificial Sequence
  plasmid
gattataagaaacattataacatattttagaactttttaactattctaaaagattaatttacatattaacatttaattat

SEQ ID NO: 125 - DNA, 75 bp, Artificial Sequence
  plasmid
taagaaacattataacatattttagaactttttaactattctaaaagattaatttacatattaacatttaattat

SEQ ID NO: 126 - DNA, 70 bp, Artificial Sequence
  plasmid
aacattataacatattttagaactttttaactattctaaaagattaatttacatattaacatttaattat

SEQ ID NO: 127 - DNA, 65 bp, Artificial Sequence
  plasmid
tataacatattttagaactttttaactattctaaaagattaatttacatattaacatttaattat

SEQ ID NO: 128 - DNA, 60 bp, Artificial Sequence
  plasmid
catattttagaactttttaactattctaaaagattaatttacatattaacatttaattat

SEQ ID NO: 129 - DNA, 55 bp, Artificial Sequence
  plasmid
tttagaactttttaactattctaaaagattaatttacatattaacatttaattat

SEQ ID NO: 130 - DNA, 50 bp, Artificial Sequence
  plasmid
aactttttaactattctaaaagattaatttacatattaacatttaattat

SEQ ID NO: 131 - DNA, 45 bp, Artificial Sequence
  plasmid
tttaactattctaaaagattaatttacatattaacatttaattat

SEQ ID NO: 132 - DNA, 40 bp, Artificial Sequence
  plasmids
ctattctaaaagattaatttacatattaacatttaattat

SEQ ID NO: 133 - DNA, 35 bp, Artificial Sequence
  plasmid
ctaaaagattaatttacatattaacatttaattat

SEQ ID NO: 134 - DNA, 90 bp, Artificial Sequence
  plasmid
tttaatataagattataagaaacattataacatattttagaactttttaactattctaaaagattaatttacatattaacatttaattat

SEQ ID NO: 135 - DNA, 90 bp, Artificial Sequence
  plasmid
aaattatattgattataagaaacattataacatattttagaactttttaactattctaaaagattaatttacatattaacatttaattat

SEQ ID NO: 136 - DNA, 90 bp, Artificial Sequence
  plasmid
aaatttataactaattaagaaacattataacatattttagaactttttaactattctaaaagattaatttacatattaacatttaattat

SEQ ID NO: 137 - DNA, 90 bp, Artificial Sequence
  plasmid
aaatttataagattaattctaacattataacatattttagaactttttaactattctaaaagattaatttacatattaacatttaattat

SEQ ID NO: 138 - DNA, 90 bp, Artificial Sequence
  plasmid
aaatttataagattataagattgtatataacatattttagaactttttaactattctaaaagattaatttacatattaacatttaattat

SEQ ID NO: 139 - DNA, 90 bp, Artificial Sequence
  plasmid
aaatttataagattataagaaacatatattcatattttagaactttttaactattctaaaagattaatttacatattaacatttaattat

SEQ ID NO: 140 - DNA, 90 bp, Artificial Sequence
  plasmid
aaatttataagattataagaaacattataagtatatttagaactttttaactattctaaaagattaatttacatattaacatttaattat

SEQ ID NO: 141 - DNA, 90 bp, Artificial Sequence
  plasmid
aaatttataagattataagaaacattataacatataaatcaactttttaactattctaaaagattaatttacatattaacatttaattat

SEQ ID NO: 142 - DNA, 90 bp, Artificial Sequence
  plasmids
aaatttataagattataagaaacattataacatattttagttgaatttaactattctaaaagattaatttacatattaacatttaattat

SEQ ID NO: 143 - DNA, 90 bp, Artificial Sequence
  plasmid
aaatttataagattataagaaacattataacatattttagaacttaaattctattctaaaagattaatttacatattaacatttaattat

SEQ ID NO: 144 - DNA, 90 bp, Artificial Sequence
  plasmid
aaatttataagattataagaaacattataacatattttagaactttttaagataactaaaagattaatttacatattaacatttaattat

SEQ ID NO: 145 - DNA, 193 bp, Artificial Sequence
  artificial construct
tataagattataagaaacattataacatattttagaactttttaactattctaaaagattaatttacatattaacatttaattatgggtaaaaactattttgaaaaatgatttatatggaattatgtttcttaaatatacaatcatgtttcatgaatacataattattttaaatgtattgggagggtcatatg

SEQ ID NO: 146 - DNA, 82 bp, Artificial Sequence
  Sequence data
attactttaaaaattaataaaaacatggtaaaatataaatcgtataaagttgtgtaatttttaaggaggtgtgttacatatg

SEQ ID NO: 147 - DNA, 25 bp, Artificial Sequence
  sequence data
tttttttttgtataaagttgtgtaa

SEQ ID NO: 148 - DNA, 48 bp, Artificial Sequence
  UTR in a plasmid
tataaaaattactttaaaaattaataaaaacatggtaaaatataaatc

SEQ ID NO: 149 - DNA, 48 bp, Artificial Sequence
  UTR in a plasmid
tataaaaaaaacttaacttcatgttaaaaacttgttaaaatataaatc

SEQ ID NO: 150 - DNA, 69 bp, Artificial Sequence
  UTR in a plasmid
tataaaaattactttaaaaattaataaaaacatggtaaaatataaatcgtataaagttgtgtaattttt

SEQ ID NO: 151 - DNA, 144 bp, Artificial Sequence
  riboswitch
  misc_feature (135)..(141): n is a, c, g, or t
tacagttatataaaaattactttaaaaattaataaaaacatggtaaaatataaatcggtaccaatacgactcactataggttccggtgataccagcatcgtcttgatgcccttggcagcaccctgctaaggaggnnnnnnnatg

SEQ ID NO: 152 - DNA, 154 bp, Artificial Sequence
  riboswitch
  misc_feature (145)..(151): n is a, c, g, or t
tacagttatataaaaattactttaaaaattaataaaaacatggtaaaatataaatcgtataaagttgtgtaatttttcaatacgactcactataggtgataccagcatcgtcttgatgcccttggcagcaccctgctaaggaggnnnnnnnatg

SEQ ID NO: 153 - DNA, 40 bp, Artificial Sequence
  riboswitch
aataaaaagcttttatgttgataaactgctttgcaaagct

SEQ ID NO: 154 - DNA, 90 bp, Artificial Sequence
  riboswitch
aataaaaagcttttatgttgataaactgctttgcaaagctctcatgtaaatgtttaatataagactactataaaattggctaattttata

(19) United States
(12) Patent Application Publication
(10) Pub. No.: US 2022/0213491 A1
(43) Pub. Date: Jul. 7, 2022

(54) GENETIC CONSTRUCT AND USES THEREOF

(71) Applicant: THE UNIVERSITY OF NOTTINGHAM, Nottingham (GB)

(72) Inventors: Nigel Minton, Nottingham (GB); Ines Canadas Blasco, Nottingham (GB); Daphne Groothuis, Nottingham (GB); Maria Zygouropoulou, Nottingham (GB)

(21) Appl. No.: 17/426,092

(22) PCT Filed: Jan. 28, 2020
(86) PCT No.: PCT/GB2020/050196

§ 371 Date: Jul. 27, 2021

(30) Foreign Application Priority Data
Jan. 28, 2019 (GB) 1901166.7

(51) Int. Cl. C12N 15/67; A61K 35/742; C12N 3/00

(52) U.S. Cl. CPC C12N 15/67; C12N 3/00; A61K 35/742

(57) ABSTRACT

A spore-forming cell comprising a first nucleotide sequence encoding a riboswitch and a transcriptional activator, wherein the riboswitch modulates translation of the transcriptional activator in response to a First inducer, and a second nucleotide sequence encoding a target gene that regulates spore formation, wherein the target gene is expressed in response to a second inducer and translation of the transcriptional activator. Also provided is a method of culturing a spore-forming cell and a method of creating a spore, as well as uses of spore-forming cells and spores.

The present invention relates to a genetic construct for use in controlling gene expression. In particular the invention relates to a genetically modified spore-forming cell comprising a genetic construct that can be used to control the expression of a gene required for spore formation. The invention further relates to a spore produced by the genetically modified spore-forming cell.

Spores are metabolically inert entities produced by some cells as a survival response to adverse environmental conditions. As such, spores are well-known for their resilient structure, capable of defying extreme chemical and physical conditions and surviving in a wide variety of environments for long periods of time. Although spores are the cause of much food spoilage, food poisoning and human diseases, their inherent resistance properties make them suitable for several beneficial applications. Of particular note is the growing use of bacterial spores in the area of biotechnology, where they have been used as probiotics, in cancer therapy, in vaccine formulations as well as in environmental bioremediation processes and biosensing systems.

The use of clostridial spores as a delivery system to treat human disease, and in particular cancer, has also been proposed. The system relies on the fact that intravenously injected clostridial spores are exclusively localised to, and germinate in, the hypoxic/necrotic tissue common to solid tumours. The therapy uses engineered clostridial spores that have the ability to produce an enzyme within the tumour which is capable of generating a toxic metabolite from a systemically introduced, non-toxic, prodrug. This strategy has been termed Clostridial-Directed Enzyme Prodrug Therapy, or CDEPT, described in Fox M E et al, Gene Therapy (1996) 3, pp 173-178. However, the risk of release from treated individuals of clostridial spores means the therapy has not yet passed the regulatory hurdles required for clinical authorisation.

While the resilient structure of spores and their mechanisms of resistance make them suitable for the aforementioned applications, they are also the cause of safety concerns associated with their use. These concerns are related to the use of genetically modified organisms (GMOs) and the risks of their release to the environment.

Firstly, the engineering and growth of recombinant strains generally makes use of antibiotic resistance genes; the potential risk of antibiotic gene transfer following release is widely recognized. Secondly, the escape of engineered organisms (e.g. Clostridia) into the environment could lead to unintentional colonization of others, even though most GMOs developed in the laboratory seem to be less fit than wildtype (WT) strains. Finally, the inherent robustness of spores results in the paradoxical situation of an unbreakable structure that needs to be “contained” or “destroyed” once used; recombinant spores could survive indefinitely in the environment after their deliberate release otherwise. Biocontainment is typically used to prevent unintended environmental release.

Physical containment, including the regulated disposal of GMO cultures, constitutes a standard procedure in research or industrial facilities. However, if GMO applications continue to expand and intend to be used in open environments, further containment, including that achieved by biological means, might be required.

A number of strategies have been developed over the last few years to provide biological containment. However, none of the known strategies have been completely successful. More importantly, none of them have specifically addressed the potential risk associated with release of recombinant spores into the environment.

The use of clostridia as gene delivery systems in human disease, and in particular cancer, has also been proposed. This is because intravenously injected clostridial spores become exclusively localised to, and germinate in, the hypoxic/necrotic tissue common to solid turnouts. This phenomenon is proposed to be exploited by endowing the clostridial cell with the ability to produce an enzyme within the tumour capable of generating a toxic metabolite from a systemically introduced, non-toxic prodrug. This strategy has been termed Clostridial-Directed Enzyme Prodrug Therapy, or CDEPT, described in Fox M E et al, Gene Therapy (1996) 3, pp 173-178. However, the risk of escape from treated individuals of clostridial spores means the therapy has little prospect of passing regulatory hurdles required for the therapy to be authorised.

There is therefore a need for an improved biological means of preventing accidental release of spores into the environment.

It is an object of the present invention to provide for the culturing of spore-forming bacteria, such as clostridia, for use in the production of a biochemical product, for use in biosensing or for use in bioremediation. An object of a specific embodiment of the invention is to enable the culture of spore-forming clostridia with a reduced need for physical containment. A further object of specific embodiments of the invention is to provide spore-forming bacteria, and spores therefrom, that can be cultured without the risk of spores being released that can sporulate and cause harm. One way to avoid, or at least reduce, the risk from unintentional sporulation of genetically engineered spores is to engineer the spores to prevent sporulation except in very specific conditions. Sporulation is a highly regulated process; thus the synthetic regulation of spore formation, therefore, requires the tight regulation of expression of “key” sporulation genes.

According to a first aspect of the invention, there is provided a spore-forming cell comprising a first nucleotide sequence encoding a riboswitch and a transcriptional activator,
wherein the riboswitch modulates translation of the transcriptional activator in response to a first inducer, and
a second nucleotide sequence encoding a target gene that regulates spore formation,
wherein the target gene is expressed in response to a second inducer and translation of the transcriptional activator.

The spore-forming cell according to the invention is highly advantageous because sporulation can be tightly regulated (i.e. sporulation may be triggered only when desired) independently of the nutritional status of the culture medium of the spore-forming cell. Due to the repressive activity of the riboswitch on the translation of the transcriptional activator, aberrant sporulation does not occur. Hence it is possible to grow the spore-forming cell in a medium free of the inducer without sporulation occurring. Thus, if a spore-forming cell according to the invention was unintentionally released into the environment it would be unable to sporulate and thus would be harmless. Sporulation will only occur when the first and/or second inducer is/are applied to the cell, which may for example, occur in vivo (when the spore forming cell is used in therapy), in vitro when maintaining a culture of spores or when preparing spore seed stocks for storage.

The present invention may provide for spore forming cells in which the regulation of sporulation is so tightly regulated that only a small amount of a (second) inducer is required to induce transcription of the target gene, which, in turn, induces spore formation.

The spore-forming cell may be a synthetic biological cell. The spore-forming cell may be a bacterium, a plant, an algae, a fungi or a protozoa. Preferably, the spore-forming cell is a bacterium.

The bacterium may be a Gram positive bacterium or a Gram negative bacterium. The bacterium may be any bacterial species, but is preferably a member of the bacterial phylum Firmicutes, which is composed of the class Clostridia (orders Clostridiales, Halanaerobiales, Natranaerobiales and Thermoanaerobacterales), the class Bacilli (orders Bacillales and Lactobacillales) and the class Mollicules (orders Acholeplasmatales, Anaeroplasmatales, Eintomoplasmatales, Haloplasmatales and Mycoplasmatales). Thus, the bacterium may be of the class Clostridia, the Bacilli or Mollicutes.

The bacterium may be within the order of Clostridiales, Halanaerobiales, Natranaerobiales, Thermoanaerobacterales, Bacillales, Lactobacillales, Acholeplasmatales, Anaeroplasmatales, Entomoplasmatales, Haloplasmatales or Mycoplasmatales. Preferably, the bacterium is within the order of Clostridiales.

Within the order Clostridiales is the genus, Clostridium. Thus, the bacteria may be of the genus, Clostridium. Preferred species are C. aceticum, C. acetobutylicum, C. aerotolerans, C. autoethanogenum, C. baratii, C. beijerinckii, C. bifermentans, C. botulinum, C. butyricum, C. cadaveris, C. cellulolyticum, C. cellulovorans, C. chauvoei, C. clostridioforme, C. colicanis, C. difficile (now renamed Clostridioides difficile), C. drakei, C. estertheticum, C. fallax, C. feseri, C. formicaceticum, C. glycolicum, C. histolyticum, C. innocuum, C. kluyveri, C. ljungdahlii, C. lavalense, C. mayombei, C. methoxybenzovorans, C. novyi, C. oedematiens, C. paraputrificum, C. pasteurianum, C. perfringens, C. phytofermentans, C. piliforme, C. ragsdalei, C. ramosum, C. roseum, C. saccharoperbutylacelonicum, C. scatologenes, C. septicum, C. sordellii, C. sporogenes, C. sticklandii, C. tertium, C. tetani, C. thermocellum, C. thermosaccharolyticum, C. tyrobutyricum, C. paprosolvens, C. saccharobutylicum, C. carboxidovorans, C. scindens, and C. autoethanogenum. as well as other acetogenic anaerobes, such as, Acetobacterium woodii, Acetonema longum, Alkalibaculum bacchi, Blautia producta, Butyribacterium methylotrophicum, Eubacterium limosum, Oxobacter pfennigii, Moorella thermoacetica, Moorella thermoautotrophica, Thermoanaerobacter kiuvi.

Within the order Bacillales are Bacillaceae which include the genera Bacillus and Geobacillus, and Staphylococcaceae, which include the genus Staphylococcus. The bacteria may be of the genus Bacillus, Geobacillus or Staphylococcus.

Preferred Bacillus species are: B. alcalophilus, B. aminovorans, B. amyloliquefaciens, B. anthracis, B. caldolyticus, B. cereus, B. circulans, B. coagulans, B. globigui, B. licheniformis, B. natto, B. polymyxa, B. phaericus, B. smithii, B. stearothermophilus, B. subtilis, B. thermoglucosidasius, B. thuringiensis and B. vulgatis.

Preferred Geobacillus species are: G. debilis, G. stearothermophilus, G. thermocatenulatus, G. thermoleovorans, G. kaustophilus, G. thermoglucosidasius, G. thermodenitrificans, G. gargensis, G. jurassicus, G. lituanicus, G. pallidus, G. subterraneas, G. tepidamans, G. thermodenitrificans, G. thermoglucosidasius, G. thermoleovorans, G. toebii, G. uzenensis and G. vulcani.

Preferred Staphylococcus species include: S. arlettae, S. aureus, S. auricularis, S capitis, S. caprae, S. carnosus, S. chromogenes, S. cohnii, S. condimenti, S. delphini, S. devriesei, S. epidermidis, S. equorum, S. felis, S. fleurettii, S. gallinarum, S. haemolyticus, S. hominis, S. hyicus, S. intermedius, S. kloosii, S. leei, S. lentus, S. lugdunensis. S. lutrae, S. lyticans, S. massiliensis, S. microti, S. muscae, S. nepalensis, S. pasteuri, S. pettenkoferi, S. piscifermentans, S. pseudintermedius, S. pulvereri, S. rostri, S. saccharolyticus, S. saprophyticus, S. schleiferi, S. sciuri, S. simiae, S. simulans, S. stepanovicii, S. succinus, S vitulinus, S. warneri and S. xylosus.

The bacterial cell may be C. acetobutylicum, C. difficile, C. beojerinckii, C. jungdahlii, C. kluyveri, C. botulinum, C. bejerinckii, C. autoethanogenum, C. pasteurianum, C. saccharobutylicum, C. carboxidovorans, C. cellulovorans, C. sporogenes, C. phytofermentans, C. ragsdalei, C. tyrobutyricum, C. perfringens, C. butyricum, C. cellulolyticum, C. formicaceticum, C. novyi, C. scatologenes, C. septicum, C. sordellii, C. sticklandii, C. tetani, C. thermocellum, C. thermosaccharolyticum, C. paprosolvens, C scindens, or C. bifermentans.

Preferably, the bacterial cell is a species selected from the group consisting of C. acetobutylicum, C. aerotolerans, C. autoethanogenum, C. baratii, C. beijerinckii, C. bifermentans, C. botulinum, C. butyricum, C. cadaveris, C. cellulolyticum, C. cellulovorans, C. chauvoei, C. clostridioforme, C. colicanis, C. difficile (now renamed Clostridioides difficile), C. estertheticum, C. fallax, C. feseri, C. formicaceticum, C. histolyticum, C. innocuum, C. kluyveri, C. ljungdahlii, C. lavalense, C. novyi, C. oedematiens, C. paraputrificum, C. pasteurianum, C. perfringens, C. phytofermentans, C. piliforme, C. ragsdalei, C. ramosum, C. roseum, C. saccharoperbutylacetonicum, C. scatologenes, C. septicum, C. sordellii, C. sporogenes, C. sticklandii, C. tertium, C. tetani, C. thermocellum, C. thermosaccharolyticum, C. tyrobutyricum, C. paprosolvens, C. saccharobutylicum, C. carboxidovorans, C. scindens, or C. autoethanogenum.

The bacterial cell may be C. phytofermentans, C. hylemonae, C. leptum, C. symbiosum, C. nexile, C. ramosum, C. bolteae, C. asparagiforme, C. methylpentosum, C. butyricum, C. sporogenes and C. scindens.

The bacterial cell may be Cupriavidus necator or metalodurans or is a cyanobacteria A nucleotide sequence according to the invention may be DNA (such as cDNA) or RNA (such as mRNA). Preferably, the first and second nucleotide sequences referred to herein are the same type of nucleotide sequence, for example, both DNA or both RNA.

Preferably, the first nucleotide sequence and the second nucleotide sequence are divergently orientated. The skilled person would appreciate that divergently orientated genes, are regulated by separate cis regulatory elements. The first nucleotide sequence and/or the second nucleotide sequence may be operatively linked to a cis regulatory element, such as a promoter. Preferably, the promotor operatively linked to the first nucleotide sequence is P_{bgaR}. Preferably, the promotor operatively linked to the second nucleotide sequence is P_{bgaL}.

A riboswitch may be an RNA molecule, such as mRNA. The riboswitch may comprise or consist of an aptamer domain, which is capable of specifically binding to an inducer, and an expression platform, which undergoes a conformational change (in response to the binding of the inducer to the aptamer domain) that promotes translation or transcription of the transcriptional activator.

The riboswitch may modulate translation or transcription of a transcriptional activator in response to contact of the aptamer domain with a (first) inducer. The riboswitch may modulate translation or transcription of the transcriptional activator, in response to contact with an inducer, by positively regulating translation or transcription of the transcriptional activator (i.e. promoting translation of the transcriptional activator) or negatively regulating translation or transcription of the transcriptional activator (i.e. inhibiting translation of the transcriptional activator).

The expression platform of the riboswitch may comprise a nucleotide sequence encoding a regulatory domain that can be used to modulate translation or transcription of the transcriptional activator. The regulatory domain may be a ribosome binding site (RBS), which is also referred to as the Shine-Dalgarno (SD) sequence. The SD sequence is complementary to the 3′ end of the 16S rRNA. In Clostridia and Bacillus the nucleotide sequence of the 3′ end of the 16S rRNA sequence may be referred to herein as SEQ ID NO. 1, as follows:

[SEQ ID NO. 1]
3′-AUCUUUCCUCCACUAGGUCGGCGUCCAAGAGGAUG-3.

A nucleotide sequence encoding a consensus SD sequence may be referred to herein as SEQ ID NO. 2, as follows:

[SEQ ID NO. 2]
5′-AAAGGAGGUGU-3′.

The consensus SD sequence may be followed by an initiation codon, most commonly AUG. In around 8% of cases the start site is GUG, whereas UUG and AUU are rare initiators present in autogenously regulated genes. Thus, the initiation codon may be AUG, GUG, UUG or AUU. The skilled person would appreciate that in the absence of a functional RBS, ribosomes are incapable of binding to mRNA, and thus incapable of being translated into a protein.

In embodiments in which the riboswitch positively regulates translation of the transcriptional activator, the regulatory domain may, in the absence of a (first) inducer, be sequestered by the expression platform, thus preventing binding of one or more ribosomes to the regulatory domain. Binding of the (first) inducer to the aptamer domain may cause the expression platform to undergo a conformational change that releases (the formerly sequestered) regulatory domain, such that one or more ribosomes can bind to the regulatory domain and thus translate the (first) nucleotide sequence into a protein.

In embodiments in which the riboswitch negatively regulates translation, the regulatory domain present in the expression platform may, in the absence of a (first) inducer, be available to bind one or more ribosomes. Binding of the (first) inducer to the aptamer may cause the expression platform to undergo a conformational change that sequesters the regulatory domain, such that one or more ribosomes cannot bind to the regulatory domain and thus cannot translate the (first) nucleotide sequence into a protein.

The riboswitch may be a naturally-occurring riboswitch or a synthetic riboswitch. A naturally occurring riboswitch may be a riboswitch responsive to adenosylcobalamin, aquacobalamin, thiamin pyrophosphate, flavin mononucleotide, s-adenosylmethionine, molybdenum cofactor, tungsten cofactor, tetrahydrofolate, s-adenosylhomocysteine, guanine, adenine, prequeuosine-1-, 2′-deoxyguanosine, cyclic di-gmp, cyclic di-amp, cyclic amp-gmp, ztp, mg²⁺, mn²⁺, f⁻, ni²⁺/co²⁺, lysine, glycine, glutamine, glucosamine-6-phosphate, azaaromatics or guanidine.

A synthetic riboswitch may be a riboswitch responsive to tetracycline; neomycin; 2,4,6-trinitrotoluene (TNT); ammeline; 5-azacytosine; theophylline; pyrimido[4,5-d]pyrimidine-2,4,-diamine (PPDA); 2-aminopyrimido[4,5-d]pyrimidin-4(3H)-one- (PPAO) or 2,6-diamino preQ0- (DPQ0).

The aptamer domain of the riboswitch may specifically bind the (first) inducer, such as, theophylline, and thus be referred to as a theophylline-responsive riboswitch. Theophylline is a purine that has high affinity for the aptamer domain of the theophylline-responsive riboswitch.

The discriminatory capacity of the aptamer with respect to related purines, which are structurally similar, is very high. For example, the aptamer of the theophylline-responsive riboswitch has a binding affinity that is 10,000-fold greater for theophylline than that of caffeine, which only differs from theophylline with respect to a methyl group located at nitrogen atom N-7. The aptamer domain specific for theophylline can be used in to create a positive or a negative regulatory riboswitch.

Thus, in one embodiment, the riboswitch may be a positive regulatory theophylline-responsive riboswitch (i.e. a riboswitch that promotes translation of the transcriptional activator). The nucleotide sequence encoding the positive regulatory theophylline-responsive riboswitch is referred to herein SEQ ID NO. 3, 4, 5, 6, 7, 8 or 9, as shown in Table 1:

TABLE 1

| | | SEQ. |
|---|---|---|
| Riboswitch | Sequence (5′ [symbol] 3′) | ID No. |
| D | GGUGAUACCAGCAUCGUCUUGAUGCCCUUGGCA | 3 |
| | GCACCCUGCUAAGGUAACAACAAGAUG | |
| E | GGUGAUACCAGCAUCGUCUUGAUGCCCUUGGCA | 4 |
| | GCACCCUGCUAAGGAGGUAACAACAAGAUG | |
| E* | GGUGAUACCAGCAUCGUCUUGAUGCCCUUGGCA | 5 |
| | GCACCCUGCUAAGGAGGCAACAACAUG | |
| F | GGUGAUACCAGCAUCGUCUUGAUGCCCUUGGCA | 6 |
| | GCACCCUGCUAAGGAGGUAACAACAUG | |
| G | GGUGAUACCAGCAUCGUCUUGAUGCCCUUGGCA | 7 |
| | GCACCCUGCUAAGGAGGUAACUUAAUG | |
| H | GGUGAUACCAGCAUCGUCUUGAUGCCCUUGGCA | 8 |
| | GCACCCUGCUAAGGAGGUGUGUUAAUG | |
| I | GGUGAUACCAGCAUCGUCUUGAUGCCCUUGGCA | 9 |
| | GCACCCUGCUAAGGAGGUCAACAAGAUG | |

The invention may comprise a nucleotide sequence encoding a riboswitch substantially as set out in SEQ ID NO. 3, 4, 5, 6, 7, 8 or 9, or a variant or fragment thereof. Preferably, the invention comprises a nucleotide sequence encoding a riboswitch substantially as set out in SEQ ID NO. 4, 7 or 8, or a variant or fragment thereof. Most preferably, the invention comprises a nucleotide sequence encoding a riboswitch substantially as set out in SEQ ID NO. 7, or a variant or fragment thereof.

An inducer may be any molecule that can specifically induce expression of a transcriptional activator as referred to herein.

The (first) inducer may induce gene expression by binding to the aptamer of a riboswitch. Thus, in one embodiment, the (first) inducer may be theophylline. However, the skilled person would appreciate that the (first) inducer may be a molecule that is capable of specifically binding to an aptamer domain, such as adenosylcobalamin; aquacobalamin; thiamin pyrophosphate; flavin mononucleotide; s-adenosylmethionine; molybdenum cofactor; tungsten cofactor, tetrahydrofolate s-adenosylhomocysteine; guanine; adenine; prequeuosine-1-, 2′-deoxyguanosine; cyclic di-gmp; cyclic di-amp; cyclic amp-gmp; ztp; mg2+; mn2+; f−; ni2+/co2+; lysine; glycine; glutamine, glucosamine-6-phosphate, azaaromatics, guanidine; tetracycline; neomycin; 2,4,6-trinitrotoluene (TNT); ammeline; 5-azacytosine; theophylline; pyrimido[4,5-d]pyrimidine-2,4,-diamine (PPDA); 2-aminopyrimido[4,5-d]pyrimidin-4(31H)-one- (PPAO) or 2,6-diamino preQ0- (DPQ0). Similarly, the skilled person would appreciate that the aptamer domain may be any domain that specifically binds to an inducer.

The second inducer may induce expression by binding to an inducible promoter (i.e. a promoter capable of promoting the expression of a gene in response to contact of the cell with an inducer). The inducer may act directly upon the promoter sequence, or may act by counteracting the effect of a repressor molecule. The inducer may be a chemical agent such as a metabolite, a protein, a growth regulator, or a toxic element, a physiological stress such as heat, wounding, or osmotic pressure, or an indirect consequence of the action of a pathogen or pest. In one embodiment, the second inducer is lactose.

In order to control sporulation of a spore-forming cell according to the invention, so that it is not accidentally released into the environment, the first and/or second inducer may be a molecule that is not generally in the environment or located extracellularly, such as the inducer of a synthetic riboswitch.

A transcriptional activator may be any agent that promotes transcription of a gene, such as the target gene. A transcriptional activator may activate/facilitate transcription by promoting interaction between RNA polymerase and DNA. A transcriptional activator may therefore be a transcription factor. A transcription factor may be a sequence specific-DNA binding protein that modulates the transcription of DNA into mRNA. A transcription factor may promote transcription by directly or indirectly recruiting RNA polymerase to the promoter of a gene to be transcribed.

The transcriptional activator may bind to a DNA-binding site specific for the transcriptional activator. The DNA-binding site may be downstream of the nucleotide sequence of the transcriptional activator, upstream or act in trans. The DNA-binding site may be upstream of the target gene, such as within the promoter of the target gene. The transcriptional activator may bind to or interact with the DNA-binding site in the absence of the second inducer. The second inducer may increase binding of the transcriptional activator to a DNA-binding site. Expression of the target gene may be leaky. Thus, the target gene may be expressed in the absence of the inducer. Expression of the target gene may be increased by binding of the second inducer.

In one embodiment, the transcriptional activator is the transcription factor, BgaR. The nucleotide sequence encoding BgaR is referred to herein as SEQ ID NO. 10, as follows:

[SEQ ID NO. 10]
ATGCAAATATTGTGGAAAAAGTATGTTAAAGAAAACTTTGAAATGAATG
TAGATGAATGTGGTATAGAACAAGGTATACCAGGATTAGGATATAACTA
TGAAGTATTGAAAAATGCTGTTATTCATTACGTAACTAAGGGATATGGA
ACTTTTAAATTTAATGGTAAGGTATATAACTTAAAACAAGGTGATATTT
TTATACTACTAAAAGGTATGCAAGTTGAGTATGTGGCTTCTATTGATGA
TCCTTGGGAATACTACTGGATAGGATTTAGTGGTTCAAATGCTAATGAG
TATTTAAATAGAACTTCTATTACTAACTCCTGTGTTGCTAATTGTGAAG
AAAACTCAAAAATTCCACAGATAATATTAAATATGTGCGAAATATCAAA
AACTTATAATCCTTCAAGATCTGATGACATACTATTACTAAAAGAACTT
TACTCATTATTGTACGCACTTATAGAAGAATTCCCAAAACCTTTTGAAT
ACAAAGATAAGGAATTACACACATATATTCAAGATGCTCTTAATTTCAT
TAATTCTAATTACATGCATAGCATAACTGTTCAAGAAATTGCTGATTAT
GTGAACTTAAGTAGAAGTTATTTATATAAAATGTTCATAAAAAACCTTG
GAATTTCTCCTCAAAGATATTTAATAAACCTTAGAATGTACAAAGCCAC
CCTTTTATTAAAAAGCACTAAACTTCCTATAGGAGAAGTCGCAAGTAGT
GTAGGTTATAGTGACTCCCTGTTATTTTCAAAAACTTTTTCAAAACATT
TTTCAATGTCTCCACTAAATTACAGAAATAATCAAGTAAATAAACCAAG
TATATAAA

Therefore, in one embodiment, the transcriptional activator is encoded by a nucleotide sequence substantially as set out in SEQ ID NO. 10, or a variant or fragment thereof.

The DNA-binding site of the transcriptional activator (e.g. BgaR) may be within the promoter of the target gene. The DNA-binding site of BgaR is preferably between positions −74 and −54 (relative to the transcriptional start site of the target gene). In one embodiment, the DNA-binding site of BgaR is referred to herein as SEQ ID NO. 11, as follows:

[SEQ ID NO. 11]
(−74) TAAGAAACATTATAACATATTTTAGAACTT (−54)

Thus, in one embodiment, the second nucleotide sequence encodes a DNA-binding site of BgaR that is substantially as set out in SEQ ID NO. 11, or a variant or fragment thereof.

The spore-forming cell referred to herein may be a transgenic cell or a genetically modified organism (GMO), such as a genetically modified bacterium. The transgenic bacterium may express a target gene that controls sporulation (see Example 4). It will be appreciated that genetically modified organisms (particularly bacteria) present safety concerns because of the risks associated with their release into the environment. In order to counteract these risks, physical containment and biocontainment may be used.

The target gene may therefore be any gene that can be used to create a transgenic organism or a GMO. Expression of the target gene may be a ‘leaky’. Preferably, the target gene is a gene that controls sporulation, preferably in bacteria.

Thus, the spore-forming cell is only able to sporulate in the presence of the first and/or the second inducer. This allows the maintenance of the cell(s), and facilitates the preparation of spore seed stocks. Subsequently, the spores can be allowed to germinate in an appropriate media and cultivated under conditions in which the inducer is absent. During growth they are able to produce a desired biochemical commodity (protein or metabolite) but they are unable to sporulate. This prevents contamination of the aerobic environment external to the actively growing anaerobic culture.

In one embodiment, the target gene is spo0A. This gene encodes the protein, Spo0A, which is a master regulator of sporulation in Bacilli and Clostridia. The nucleotide sequence encoding Spo0A C. sporogenes is referred to herein as SEQ ID NO. 12, as follows:

ATGGAAGAAACAAAGATCAATGTTATCATTGCAGATGATAATAAGGAAT
TTTGTAACATATTAAGTGATTATTTGTTAAATCAAAGAGACATAATGGT
TACAGGTATAGCTAATGATGGAGTAGAAGCTCTTAAATTGGTGGAAGAA
AAAAAACCAGATTTAATAATACTAGATATAATAATGCCTCATTTAGATG
GATTAGGAGTTTTAGAAAAATTAAATAGTATAGATATTACTCCTATGCC
AAGGGTAATTGTTTTGTCTGCTGTAGGACAGGATAAGATTACTCAAAGG
GCTATAAACTTAGGTGCAGATTATTATGTAGTTAAACCTTTTGATATGG
ACGTTTTTACCAAGAGAATAAGACAAATGTTTAACAATACCATAAGTAG
CGAAGAAGTTAGAAGACCAGTAGTAATGCCAGAAACATGTTCACAAGAA
AAAATATCATTATCCAAAAATGAACCTGCAGATTTAGAACAAGAAATAA
CAGATATTATACATGAAATAGGTGTACCAGCTCATATAAAAGGATATAT
GTATTTGAGAGAAGCTATAACTATGGTAGTTAATGACATGGAACTTTTA
TCCGCAGTGACAAAGGAGTTGTACCCTTCAATAGCTAAAAAATATAATA
CTACAGCAAGTAGAGTAGAAAGAGCTATAAGACATGCTATAGAAGTAGC
TTGGTCAAGAGGTCAAGTTGAAACTATAAACAATATATTTGGGTATACT
ATAAATAATGGAAAGGGAAAACCAACAAATTCTGAGTTTATTGCAATGG
TAGCTGATAAATTAAGACTAAAAAATAGAGTTTGCTAA

[SEQ ID NO. 12]

Therefore, in one embodiment, the target gene is encoded by a nucleotide sequence substantially as set out in SEQ ID NO. 12, or a variant or fragment thereof.

The skilled person will appreciate that other organism may express their own Spo0A protein which may be the same, similar or different to that of SEQ ID NO. 12.

The target gene alternatively may be any of the following genes, which control sporulation in bacteria (particularly C. sporogenes): spoIID, divIVA, spoIIM, ftsk, ftsA2, clpx, spoIIE, spoIIR, sigG, sigE, sigK, sigF, spoIIAB, spoIIAE, spoIIIAA, cbij, cipP1 or mmmE. The target gene may be any gene which controls sporulation in bacteria.

The first nucleotide sequence and the second nucleotide sequence may be encoded by at least one genetic construct. Thus, the spore-forming cell may comprise at least one genetic construct having a first nucleotide sequence encoding a riboswitch and a transcriptional activator.

In an alternative embodiment, the spore-forming cell comprises two genetic constructs, A first genetic construct may comprise a first nucleotide sequence encoding a riboswitch and a transcriptional activator, wherein the riboswitch modulates translation or transcription of the transcriptional activator in response to a first inducer, and a second genetic construct may comprise a second nucleotide sequence encoding a target gene, wherein a second inducer is capable of inducing expression of the target gene and the transcriptional activator promotes transcription of the target gene.

The target gene may be operably linked to a cis regulatory element. Thus, the spore-forming cell or a genetic construct according to the invention may encode one or more cis regulatory elements. A cis regulatory element may be a promoter, a DNA-binding region within the promoter (e.g. O2), an operator or an RBS.

The invention may further comprise cis regulatory element, O2. O2 may be a consensus motif of the DNA-binding region of the transcriptional activator (e.g. BgaR), such as SEQ ID NO. 11. The cis regulatory element may be present within the promoter of the target gene. For example, O2 may be at position −10 upstream of the target gene. In one embodiment, O2 is referred to herein as SEQ ID NO. 13, as follows:

[SEQ ID NO. 13]
AGAAACATTATAACATATTT

Thus, the invention may further comprise a nucleotide sequence substantially as set out in SEQ ID NO. 13, or a variant or fragment thereof.

Alternatively the cis regulatory element may be O1.

According to another aspect, there is provided a method of culturing a spore-forming cell, the method comprising culturing the spore-forming cell according to the invention in the absence of a first and/or second inducer.

The spore-forming cell may be contacted with a first inducer and a second inducer in order to induce the formation of a spore. Contact with the first inducer and the second inducer may or may not occur simultaneously.

According to another aspect, there is provided a method of creating a spore, the method comprising contacting the spore-forming cell of the invention with a first inducer and a second inducer.

Contact with the first inducer and the second inducer may or may not occur simultaneously.

According to another aspect, there is provided a spore obtained or obtainable from the method according to the invention.

According to another aspect, there is provided the spore-forming cell or spore according to the invention for use in therapy.

According to another aspect, there is provided the spore-forming cell or spore according to the invention for use in treating or preventing treating diseases and disorders associated with the gut microbiome, such as a C. difficile infection (CDI), Irritable Bowel Disease (IBD), or cancer (bowel cancer).

The gut microbiome plays a pivotal role in gut health. Its constituent bacteria are involved in food digestion, produce essential vitamins and enzymes, provide immunity against pathogens, and help regulate the inflammatory response. Disruption of the microbiome can result in Irritable Bowel Disease (IBD) as well as opportunistic infection from antibiotic resistant pathogens such as C. difficile. Both IBD and C. difficile infection (CDI) have a huge social impact since they are recurring and debilitating diseases that affect millions of people worldwide. The economic burden on healthcare for IBD alone is estimated to be $60B. CDI is a significant clinical problem, particularly in the elderly resulting in 10-fold more deaths than MRSA. The annual cost for managing CDI is approx. is $1B in the USA and $5B in the EU. There is a major unmet need for alternative therapies for IBD and CDI.

The modified spore-forming cell of the invention may be used to treat IBD and/or CDI through the delivery of anti-microbial and anti-inflammatory peptides. Pharmaceutical spore doses of clostridial strains engineered to produce the requisite peptides may be prepared in the ‘laboratory flask’ by growth of the cell line in the presence of inducer. These may be delivered to the microbiome through the oral route. Following germination in the gut and conversion of spores to vegetative cells, they will be unable to sporulate again in the absence of the requisite inducers, thereby preventing shedding of spores and environmental release.

Several Clostridium species are indigenous to the healthy GI microbiota of mammals, principally those of Clostridium clusters IV and XIVa (the Clostridium coccoides and Clostridium leptum groups, respectively). As reviewed by Cartman S T, Future Microbiology 2011, 6, pp. 969-971, these groupings have been shown to facilitate anti-inflammatory immune responses in mice by promoting the accumulation and activity of regulatory T cells and can provide resistance to experimentally induced IBD. Indeed, Clostridium clusters IV and XIVa are less abundant in faecal samples from IBD patients, compared with those from healthy individuals indicating that probiotic administration of GI associated Clostridium species may alleviate the symptoms of IBD and possibly other autoimmune diseases Interestingly, Clostridium butyricum MIYAIRI 588, a member of Clostridium cluster I, is a recognized probiotic, which has approval in the European Union for use as an animal feed supplement (Aquilina G et al., European Food Safety Authority Journal 2011 9(1), pp. 1951) and has been reported to protect against antibiotic associated diarrhoea and to be antagonist against Escherichia coli O157:H7 and Clostridium difficile (Cartman S T, Future Microbiology 2011 6, pp. 969-971). The inhibitory effects against the later are likely in part due to production of a bacteriocin active against C. difficile (Nakanishi S and Tanaka M. Anaerobe (2010) 16, pp. 253-257).

Accordingly, those Clostridium species native to the gut may be engineered to deliver anti-microbial, or anti-inflammatory, peptides to counter CDI and IBS, respectively. For instance, the antagonistic activity of clostridia against C. difficile could be enhanced by engineering the cell to produce small peptides, such as Coprisin or similar. This peptide is active against C. difficile but not other members of the microbiota, such as Lactobacillus and Bifidobacterium (Antimicrobial Agents Chemother 2011; 55:4850-4857). Candidate strains for engineering could include those Clostridium shown to be present in the microbiome (Junjie Qin et al., Nature 2010 464, pp 59-65) by metagenome sequencing, such as C. phytofermentans, C. hylemonae, C. leptum, C. symbiosum, C. nexile, C. ramosum, C. bolteae, C. asparagiforme, C. methylpentosum, C. butyricum, C. sporogenes and C. scindens.

According to another aspect, there is provided the spore-forming cell or spore according to the invention for use in treating or preventing a solid tumour. Current systems for delivering therapeutic genes to tumours suffer from a number of serious deficiencies, most notably a lack of specificity for cancer cells. An innovative solution to the problem uses the spores of a harmless, nonpathogenic Clostridium species. Intravenously injected clostridial spores localise to, and exclusively germinate in, the hypoxic regions of solid tumours. The spores are incapable of germinating in healthy tissue. Clostridia may be engineered to produce a variety of therapeutic proteins with anti-tumour activity. Considerable research efforts have been invested into clostridial-directed enzyme prodrug therapies (CDEPT), whereby Clostridia are equipped with prodrug converting enzymes (PCEs) that can metabolize nontoxic prodrugs into toxic derivatives (Minton N P Nat Rev Microbiol. 2003; 1(3):237-42). Beyond CDEPT, clostridia have also been engineered to deliver desired antibodies and immunotherapeutic messengers. Moreover, to improve the utility of clostridial-based therapies, the incorporation of an imaging functionality has been explored.

PCEs include: cytosine deaminase (CodA, encoded by the E. coli codA gene) catalyses the conversion of 5-fluorocytosine (5FC) into 5-fluorouracil (5FU); Nitroreductases, a large family of nicotinamide adenine dinucleotide (phosphate) [NAD(P)H]-dependent flavoenzymes that catalyse the reduction of nitro groups, and; carboxypeptidase G2 (CPG2) which cleaves the C-terminal glutamate moiety of folate-based compounds which are essential in a number of intracellular functions and primarily in DNA synthesis. To that effect, CPG2 can be paired with glutamated benzoyl nitrogen mustard prodrugs whose CPG2-mediated metabolism would yield cytotoxic nitrogen mustard derivatives. Antibodies include anti-Hypoxia Inducible Factor 1 alpha (HIF1a) antibody and anti-Vascular Endothelial Growth Factor (VEGF) antibody. Immunotherapeutic messengers include: interleukin-2 (IL2) and interleukin-12 (IL12); interferon alpha (IFNα); granulocyte-macrophage colony-stimulating factor (GM-CSF) and granulocyte colony-stimulating factor (G-CSF), and; Tumour Necrosis Factor alpha (TNFα) (Maria Zygouropoulou, Aleksandra Kubiak, Adam V. Patterson and Nigel P Minton. “Genetic engineering of clostridial strains for cancer therapy Microbial Infections and Cancer Therapy”. In: Microbial Infections and Cancer Therapy. Eds, A M Chakrabarty & A M Fialho. pp. 73-121. Pan Stanford Publishing, USA, 2018).

Another aspect of the invention provides a modified spore forming cell for use in preventing the contamination of production facilities with spores during the manufacture from Clostridium of a biochemical, protein or other commercial product. Various clostridia are exploited in the production of numerous chemicals and fuels, as well as in the manufacture of proteins and enzymes, the latter of which me be used in the treatment of human disorders. Prominent examples are those saccharolytic clostridia that are able to convert various carbon feedstocks into the chemical solvents acetone, ethanol and butanol, typified by C. acetobutylicum and C. beijerinckii, and pathogenic species, such as C. botulinum, C. histolyticum, C. perfringens and C. difficile, which produce proteinaceous toxins with therapeutic applications. The latter include botulinum neurotoxin (BoTox), which are used to treat aberrant muscular dysfunctions and other forms of cellular disorders resulting from hypersecretion (Münchau A and Bhatia K P, British Mecial Journal 2000. 320, pp. 161-165), as well as collagenase from C. histolyticum that can be used for the treatment of Dupuytren's contracture and as an injectable medicine for treatment of Peyronie's disease (Alipour et al., Asian Pacific Journal of Tropical Biomedicine, 2016 6, pp. 975-981).

Production process that use unmodified clostridial process strains are disadvantaged by the fact that the production facility becomes contaminated with spores which are difficult to eliminate as a consequence of their resistance to all manner of physical and chemical agents. This can compromise the use of a fermentation reactor, for another purpose. A strain modified according to the invention would be unable to sporulate because the inducers (theophyline and lactose) would be omitted from the fermentation media. In contrast, during the handling of the strain for maintenance purposes prior to inoculation of the reactor, the inducers would be included in the media ensuing that spores are made. This is especially important to anaerobic organisms like Clostridium, as vegetative cells die on exposure to oxygen. Spores are resistance. It is therefore, easier to maintain stock cultures that sporulate. Indeed, strains may be maintained as a spore stock.

According to one aspect of the invention, there is provided (a nucleotide sequence encoding) a riboswitch having positive regulatory activity.

A riboswitch having positive regulatory activity may comprise a nucleotide sequence substantially as set of in SEQ ID NO. 3, 4, 5, 6, 7, 8 or 9, or a variant or fragment thereof.

According to another aspect, there is provided a genetic construct comprising or consisting of a riboswitch according to the invention.

Advantageously, the riboswitch and genetic construct according to the invention may be used to tightly regulate the expression of a target gene, particularly a target gene that exhibits leaky expression.

According to another aspect of the invention, there is provided a genetic construct comprising or consisting of a nucleotide sequence encoding a riboswitch and a transcriptional activator,
wherein the riboswitch modulates translation of the transcriptional activator in response to an inducer and the transcriptional activator promotes transcription of a target gene.

The construct according to the invention is advantageous because it may be used to reduce basal translation of an mRNA molecule encoding a transcriptional activator. Furthermore, the construct may be introduced into an inducible system comprising a target gene, whose transcription is promoted by the transcriptional activator, in order to add a layer of control over basal transcription and/or translation of the target gene (i.e. transcription and/or translation of the target gene in the absence of an inducer or an endogenous activating signal).

According to another aspect of the invention, there is provided at least one genetic construct comprising or consisting of:
a first nucleotide sequence encoding a riboswitch and a transcriptional activator,
wherein the riboswitch modulates translation of the transcriptional activator in response to a first inducer, and
a second nucleotide sequence encoding a target gene,
wherein the target gene is expressed in response to a second inducer and translation of the transcriptional activator.

The genetic construct according to the invention is advantageous because it provides tight control over an inducible system comprising a target gene whose transcription is regulated/promoted by a transcription activator, such as a transcription factor. The control may occur at both the transcriptional level (via the second inducer) and the translational level (via the first inducer). Consequently, the target gene is highly repressed in the absence of any inducers. Thus, little or no expression of the target gene occurs in the absence of an inducer.

It will be appreciated that the at least one genetic construct may be two genetic constructs, wherein a first genetic construct may comprise a first nucleotide sequence encoding a riboswitch and a transcriptional activator, wherein the riboswitch modulates translation of the transcriptional activator in response to a first inducer, and a second genetic construct comprises a second nucleotide sequence encoding a target gene, wherein the target gene is expressed in response to a second inducer and translation of the transcriptional activator.

The genetic construct(s) disclosed herein may be in the form of an expression cassette, which may be suitable for expression of the nucleotide sequence(s) in a cell or a genetic element, which may be used to create a synthetic biological cell, tissue or organism. The genetic construct of the invention may be introduced into a host cell with or without a vector.

The genetic construct(s) according to the invention may be used to regulate an inducible system in which in an undesirable level of transcription and/or translation of the target gene occurs in the absence of an inducer (i.e. high levels of basal expression, which may be referred to as ‘leakiness’). This is achieved, as shown in Examples 3 and 4, without compromising the dynamic range (the ratio between level of expression in the presence and absence of an inducer) or maximal levels of expression of the target gene.

The genetic construct(s) according to the invention may be capable of reducing ‘leakiness’ (i.e. basal transcription and/or translation of a target gene) by at least 10%, 20%, 30%, 40%, 50%, 60%, 70%, 80% or 90%. The genetic construct according to the invention may be capable of reducing leakiness by 100%.

The genetic construct(s) may be introduced into the host cell by using any suitable means, such as endocytic uptake, microinjection, ballistic bombardment, a particle gun, electroporation, transduction, transfection, infection or cell fusion. Preferably, the genetic construct(s) is/are introduced into the cell by using a vector.

According to another aspect of the invention, there is provided a vector comprising the genetic construct(s) of the invention.

The vector may be a recombinant vector. The vector may be a virus, a virus-like particle, a plasmid, a cosmid, a phage, a transposon or a liposome.

According to another aspect of the invention, there is provided a host cell comprising a genetic construct or a vector according to the invention.

The host cell may be a spore-forming cell, a bacterium, a plant, an algae, a fungi or a protozoa. Preferably the host cell is a spore-forming bacterial cell.

According to another aspect of the invention, there is provided a method of inducing translation, in a cell, of an RNA molecule encoding a (first) nucleotide sequence, the method comprising: transforming a host cell with a genetic construct or a vector according to the invention to create a transformed host cell; and contacting the transformed host cell with a (first) inducer to induce translation of the RNA of the (first) nucleotide sequence.

According to another aspect of the invention, there is provided a method of inducing expression, in a cell, of a target gene, the method comprising:
transforming a host cell with a genetic construct or a vector according to the invention to create a transformed host cell; and
contacting the transformed host cell with a first inducer and a second inducer to induce expression of the target gene in the transformed host cell.

According to another aspect, there is provided use of a genetic construct, a riboswitch or a vector according to the invention to positively regulate translation of a gene.

The term ‘in response to’ can refer to in response to contact of the cell, spore, genetic construct, vector or riboswitch according to the invention.

The term ‘expression’ can refer to transcription of a gene and/or translation of an mRNA molecule encoded by a gene.

An ‘inducible system’ can refer to a system that only expresses a target gene in the presence of an inducer (i.e. in an ‘ON’ state). In the absence of the inducer, the system is in an ‘OFF’ state.

The term ‘modulate(s)’ can refer to ‘inhibition’ and/or ‘enhancement’. The term ‘modulate(s)’ can refer to ‘prevention/cessation’ and/or ‘initiation/induction’.

A ‘variant or fragment thereof’ can be a derivative of (i) the riboswitch, which is capable of undergoing a conformational change that modulates translation of the transcriptional activator, (ii) the transcriptional activator, or (iii) the target gene, as referred to herein.

A ‘synthetic biological system’ can refer to a biological system comprising components (e.g. tissues, cells, organelles, regulatory elements, transcription machinery, translation machinery) that does not already exist in nature.

A ‘synthetic biological cell’ can refer to a cell that does not exist in nature, such as a transgenic cell or a genetically modified cell.

It will be appreciated that the invention extends to any nucleic acid or peptide or variant, derivative or analogue thereof, which comprises substantially the amino acid or nucleic acid sequences of any of the sequences referred to herein, including variants or fragments thereof.

The terms “substantially the amino acid/polynucleotide/polypeptide sequence”, “variant” and “fragment”, can be a sequence that has at least 40% sequence identity with the amino acid/polynucleotide/polypeptide sequences of any one of the sequences referred to herein, for example 40% identity with the gene identified as SEQ ID No. 1, or 40% identity with any polypeptides disclosed herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to is also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein. The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (Hi) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance. Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences is then calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (it) inserting the values of N and T into the following formula:—Sequence Identity (N/T)*100.

Alternative methods for identifying similar sequences will be known to those skilled in the art. For example, a substantially similar nucleotide sequence will be encoded by a sequence which hybridizes to the sequences shown in SEQ ID Nos. 1 to 7, or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from any of the sequences disclosed herein.

Due to the degeneracy of the genetic code, it is clear that any nucleic acid sequence could be varied or changed without substantially affecting the sequence of the protein encoded thereby, to provide a variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids. All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

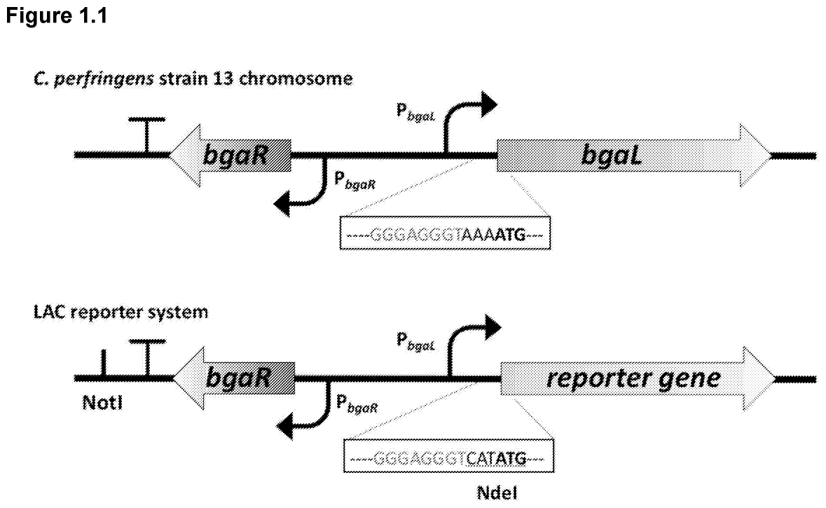

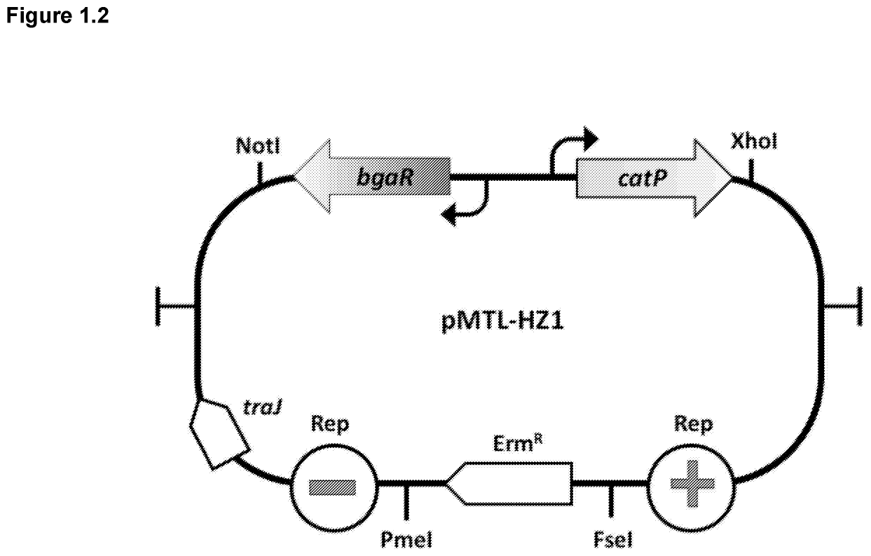

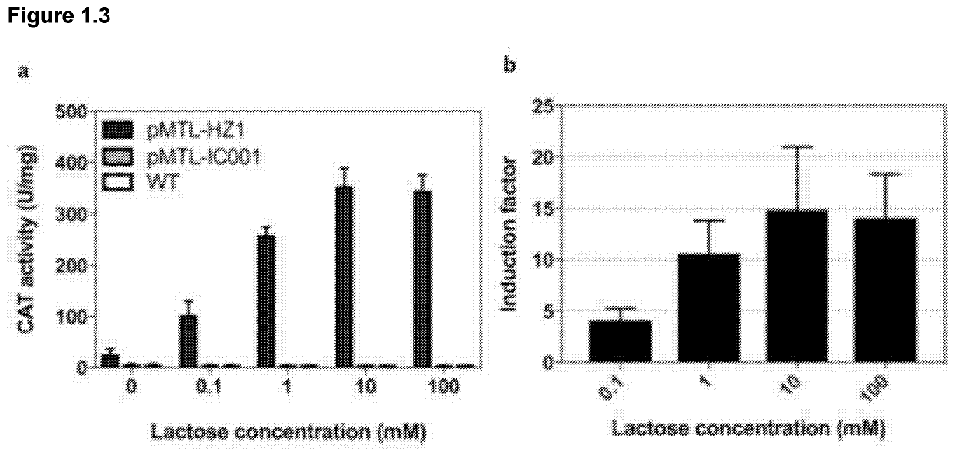

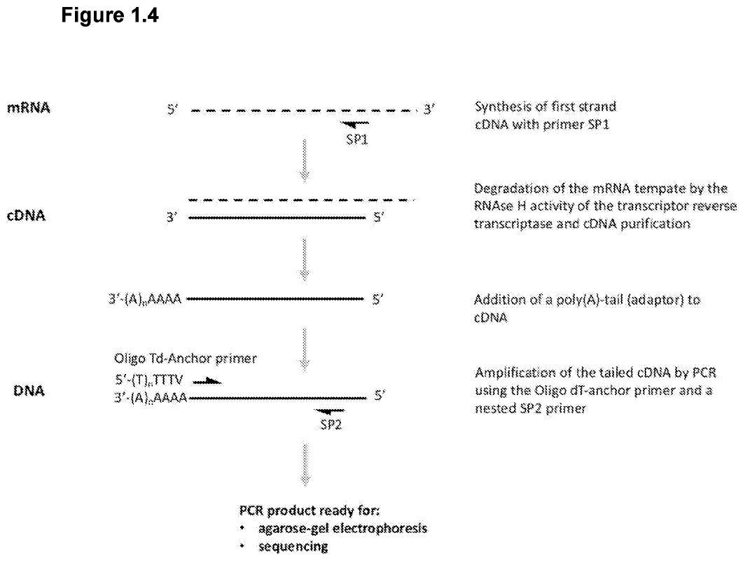

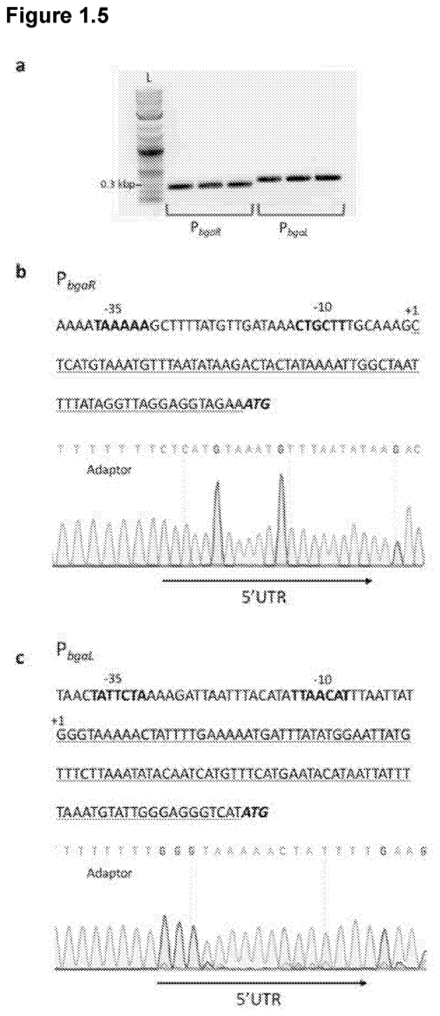

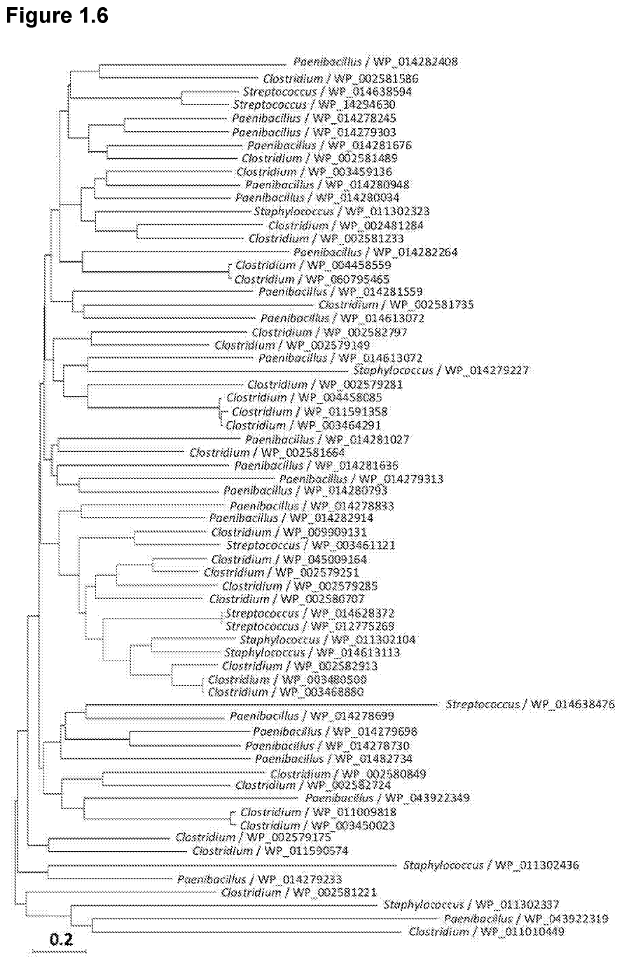

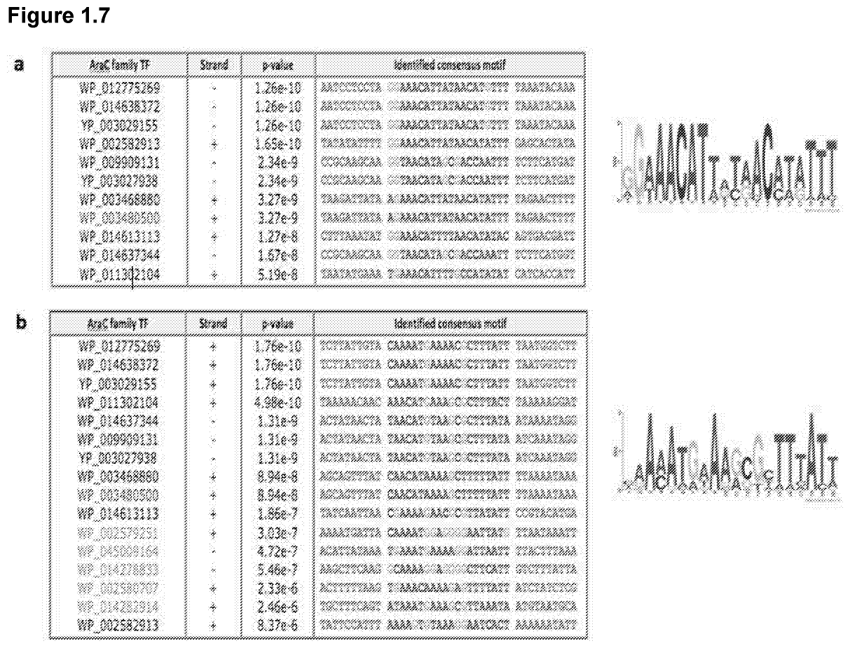

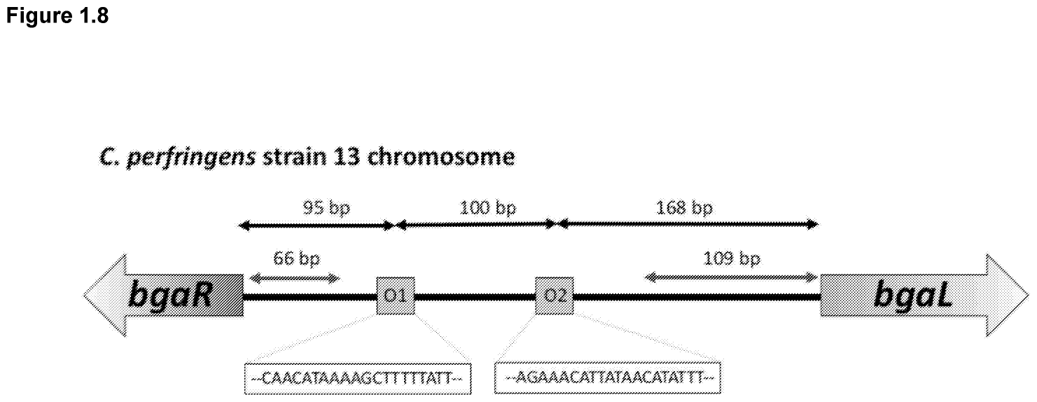

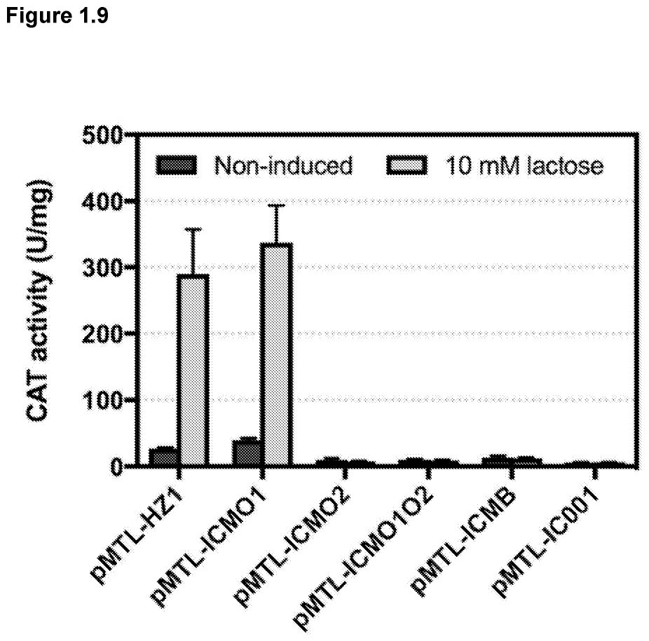

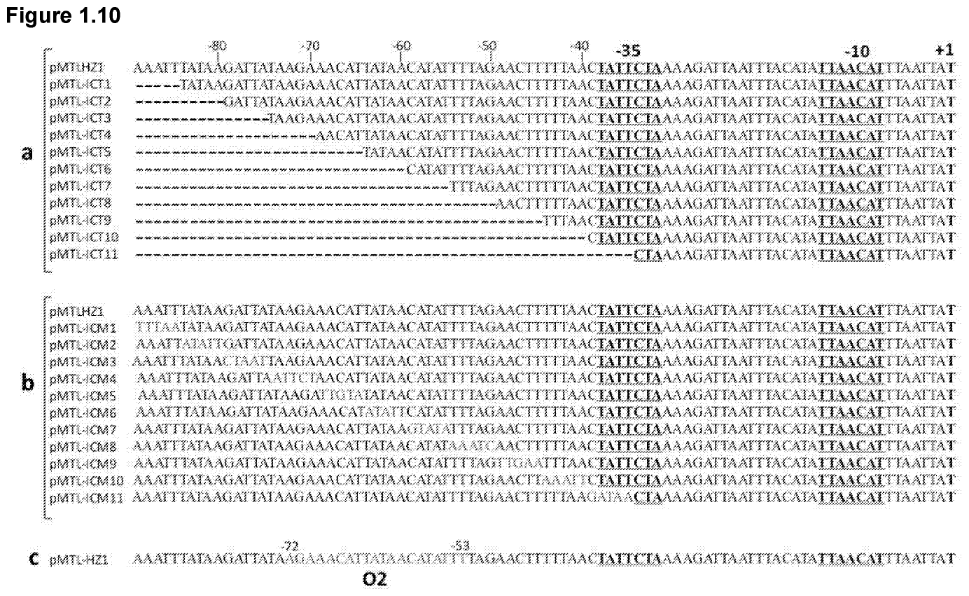

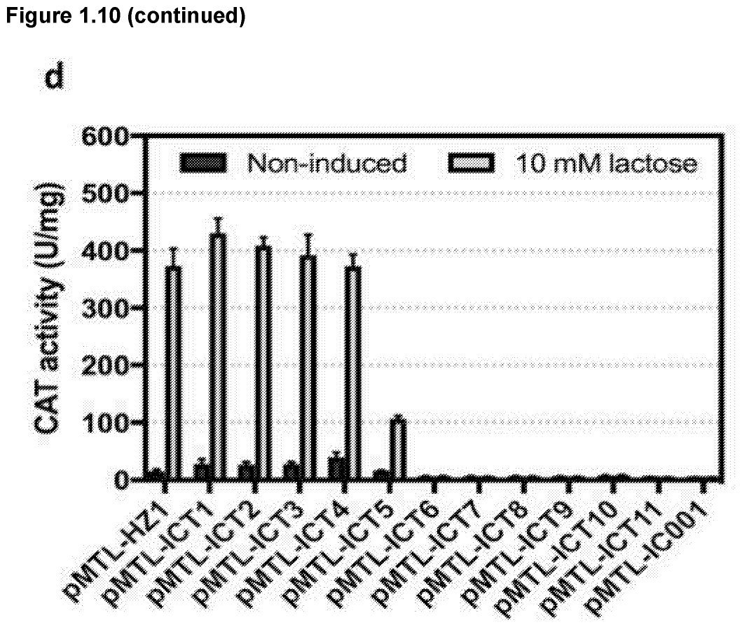

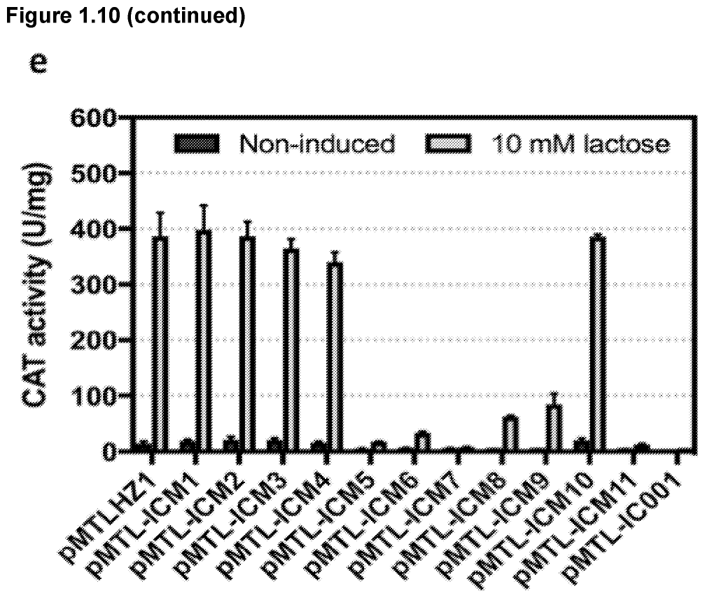

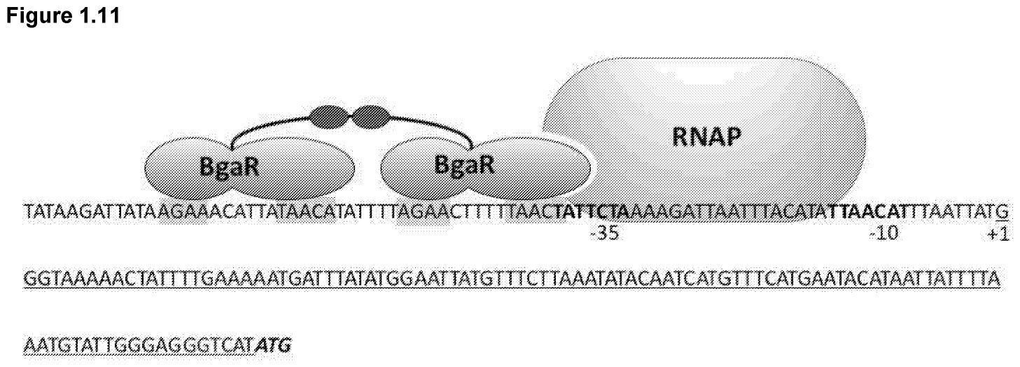

FIG. 1.1 shows a schematic representation of the lactose-inducible (LAC) system from C. perfringens strain 13. The system includes the transcriptional activator gene—bgaR—and the divergently oriented bgaL gene, which encodes a β-galactosidase involved in lactose metabolism. As a reporter system, the transcriptional activator, bgaR, and the corresponding inducible promoter, PbgaL, are flanked by the restriction sites NotI/NdeI for easy cloning into the modular pMTL8000 series vector;

FIG. 1.2 shows a schematic representation of pMTL-HZ1. The reporter gene catP is placed under the control of the LAC system. (−) is the Gram-negative origin of replication. traJ encodes the TraJ protein, needed for conjugal transfer. (+) is the Gram-positive origin of replication;

FIG. 1.3 shows a LAC system dose-dependent response. (a) Response of the LAC system in C. sporogenes to different concentrations of lactose. Pre-cultures of cells containing the plasmid pMTL-HZ1 were inoculated into fresh TYG medium to a starting OD600≈0.05; each culture was split into five equal volumes, and each volume was then supplemented with different concentrations of lactose (0, 0.1, 1, 10 and 100 mM). The promoter-less vector, pMTL-1C001, and WT were used as negative controls. (b) The activation ratio at each concentration was calculated by dividing the value of CAT activity measured in the presence of the inducer by the value of that in the absence of the inducer. Error bars represent the standard deviation of three independent biological replicates;

FIG. 1.4 shows a schematic illustration of the 5′ RACE procedure. In this study, SP1 and SP2 refer to the catP specific primers 1 (IC263-r) and 2 (IC264-r), respectively. SP1 and SP2 aligned 580 and 245 bp downstream of the translational start site, respectively. PCR products were sequenced with a third nested catP specific primer, SP3 (IC265-r), which aligns 190 nucleotides downstream of the translational start site;

FIG. 1.5 shows a 5′ RACE experiments on PbgaR and PbgaL. (a) The agarose gel of PCR amplifications of three independent DNA samples (from each promoter) that were reverse-transcribed from mRNA template according to the 5′ RACE protocol. cDNA transcription was performed with a primer aligning 250 bp downstream of the translational start site. Extension PCR was performed with a primer aligning to the adaptor at the 3′ end of the cDNA (complementary to the 5′ UTR of the mRNA) and a primer aligning 190 bp downstream of the translational start site, producing a product of at least 200 bp. L is 2-log ladder. (b) Sequence of PbgaR and 5′ RACE sequencing results. (c) Sequence of PbgaL and 5′ RACE sequencing results. In both cases, the sequence is the reverse complementary DNA of the adaptor. The putative −10 and −35 boxes were annotated manually in agreement with the determined TSS (+1). The translational start site is in bold and italicised. The 5′ UTR is underlined;

FIG. 1.6 shows a phylogenetic tree of AraC/XylS-family TF. Neighbour-Joining tree with 79 homologous AraC/XylS-family TFs from species belonging to the division Firmicutes. The tree was aligned based on the respective protein sequences. Branch lengths correspond to the relative divergence in amino acid sequences. BgaR, with accession number WP_003480500 is highlighted in red. The TFs most related to BgaR in phylogeny (black branch) were used for the identification of consensus motifs;

FIG. 1.7 shows motifs identified within 300 bp upstream of the genes encoding the AraC/XylS-family TFs. (a) Motif O1, found in 11 out of the 18 sequences selected. (b) Motif O2, found in 16 out of the 18 sequences selected. BgaR, with accession number WP_003480500, is highlighted in red. All sequences containing O1, also contained O2. In only 5 cases, highlighted in blue, was O2 present in the absence of O1;

FIG. 1.8 shows a schematic representation of the LAC system and the hypothetical operator sequences O1 and O2. Distances calculated from the position of the 10^{th} base of the 20 bp hypothetical operator sequences. Red arrows indicated the length of the 5′ UTRs;

FIG. 1.9 shows LAC mutations O1, O2 and O1/O2. Plasmid-based CAT assay of C. sporogenes strains expressing catP under the control of the LAC system (pMTL-HZ1) and the former system lacking either the hypothetical operator sequences O1 or O2 (pMTL-ICMO1 and pMTL-ICMO2, respectively) or both operator sequences (pMTL-ICMO1O2). The vector pMTL-ICMB contains the LAC system with a deletion of the entire bgaR gene. The promoter-less reporter plasmid, pMTL-IC001, was used as negative control. Cells containing the designated plasmids were cultivated in TYG medium lacking lactose as well as a culture to which lactose (10 mM) was added when they had reached the early exponential growth phase (OD600≈0.5). CAT activity was determined on cell lysates derived from stationary phase cultures. Error bars represent the standard deviation of three biological replicates;

FIG. 1.10 shows a LAC stepwise mutations and truncations. Analysis of the intergenic region of the LAC system by CAT measurements of C. sporogenes strains carrying pMTL-HZ1-derived mutant plasmids. The nucleotide sequences of the WT (pMTL-HZ1) and the mutants are shown. (a) DNA sequence of the LAC system variants with truncated intergenic region. (b) DNA sequence of the LAC system variants with blocks of five nucleotide substitutions (highlighted in red). (c) Sequence of pMTL-HZ1 intergenic region with the consensus motif O2 highlighted in red. (d) CAT results of variants with truncated 5′ UTR (sequences from (a)). (e) CAT results of variants with five nucleotide substitutions (sequences from (b)). In both cases, the promoter-less reporter plasmid, pMTLIC001, was used as negative control. Cells containing the designated plasmids were cultivated in TYG medium in the presence and absence of 10 mM lactose; induction took place when cultures had reached early exponential growth phase (OD600≈0.5). CAT activity was determined on cell lysates derived from stationary phase cultures. Error bars represent the standard deviation of three biological replicates;

FIG. 1.11 shows hypothetical BgaR binding sites and hypothetical regulation of the LAC system. In the presence of lactose (red circles), BgaR dimerizes and each monomer binds to a 15-bp half-site containing similar (but not identical) motifs. Perfect repeats of the hypothetical BgaR binding site AGAA-N6-TAACA-N6-AGAA-N5-TAACT are highlighted in blue. Imperfect repeats are highlighted in green. The 5′UTR sequence is underlined. The translational start site is in bold and italicised. According to the position of the putative −10 and −35 elements (in quite proximity to RNAP), activation of PbgaL could occur via direct interaction with the RNAP or by creating a conformational change in the DNA that increases the affinity between the RNAP and the DNA;

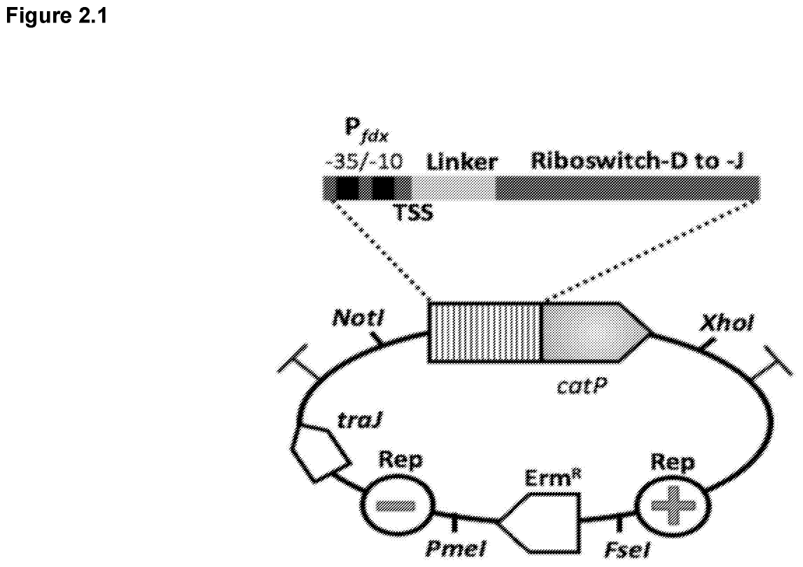

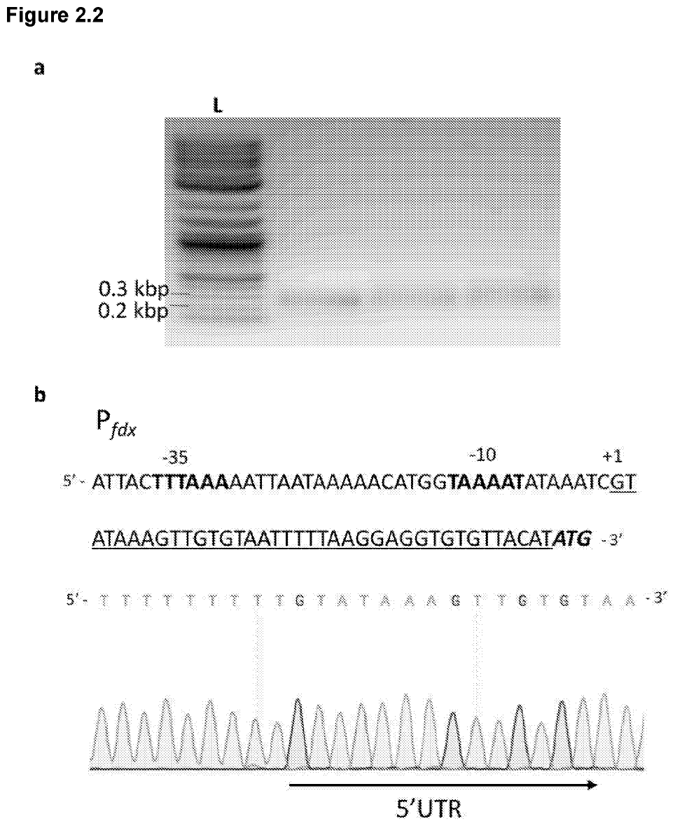

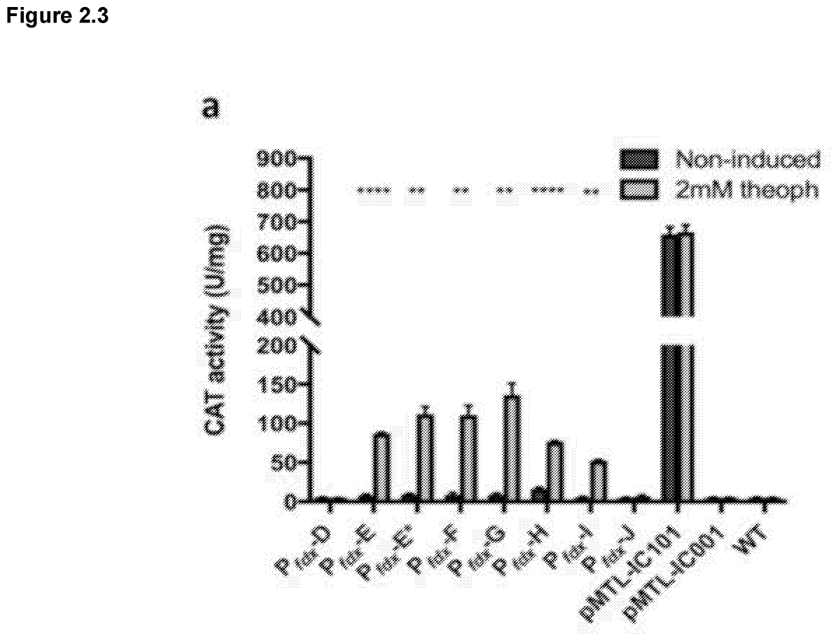

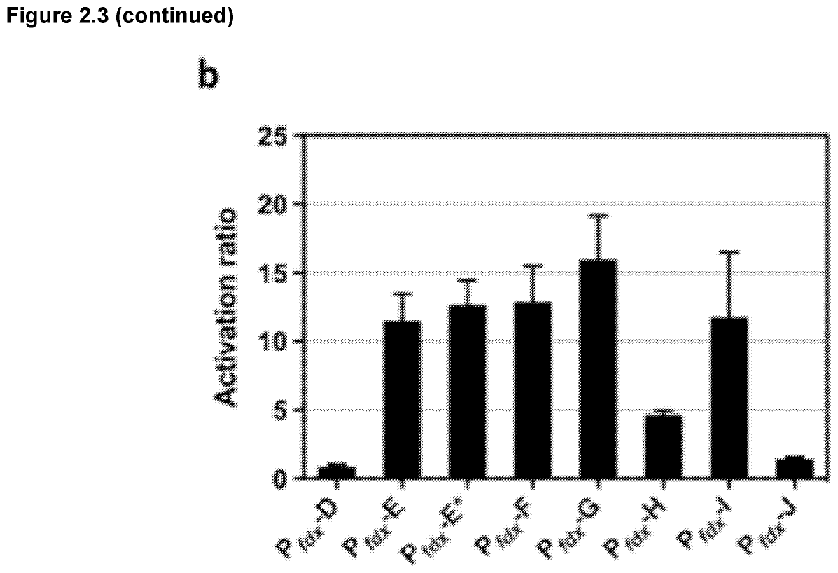

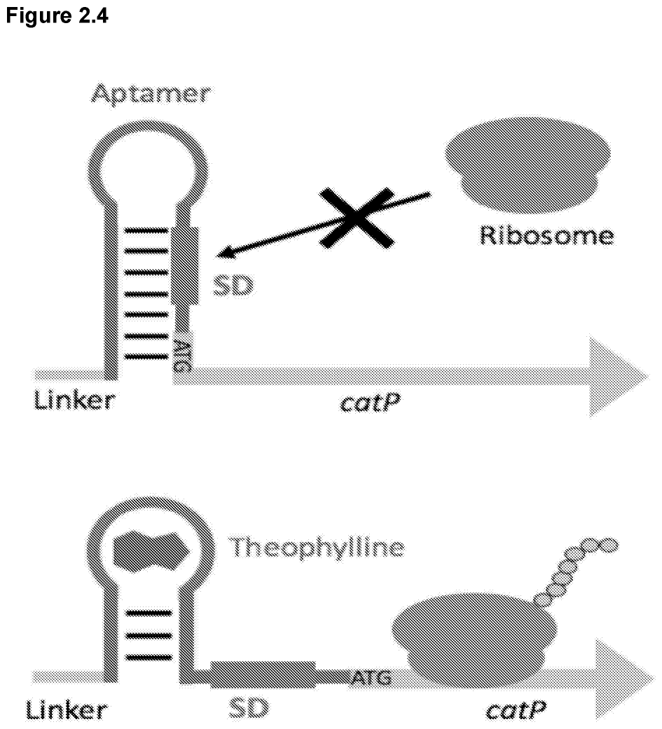

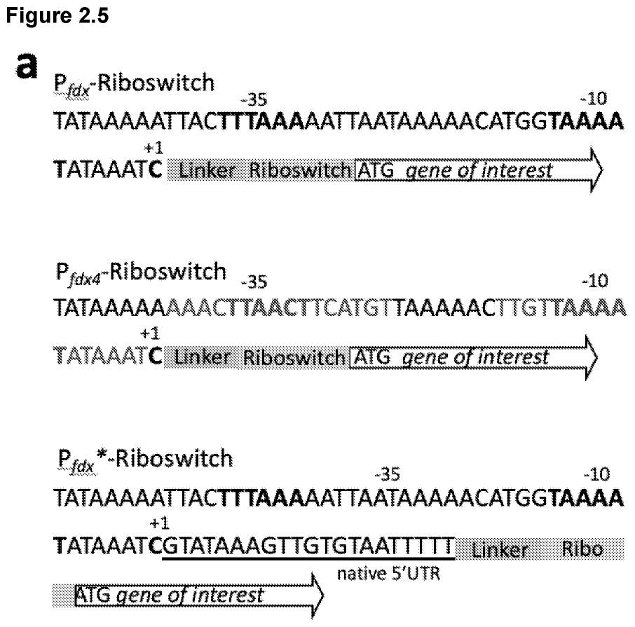

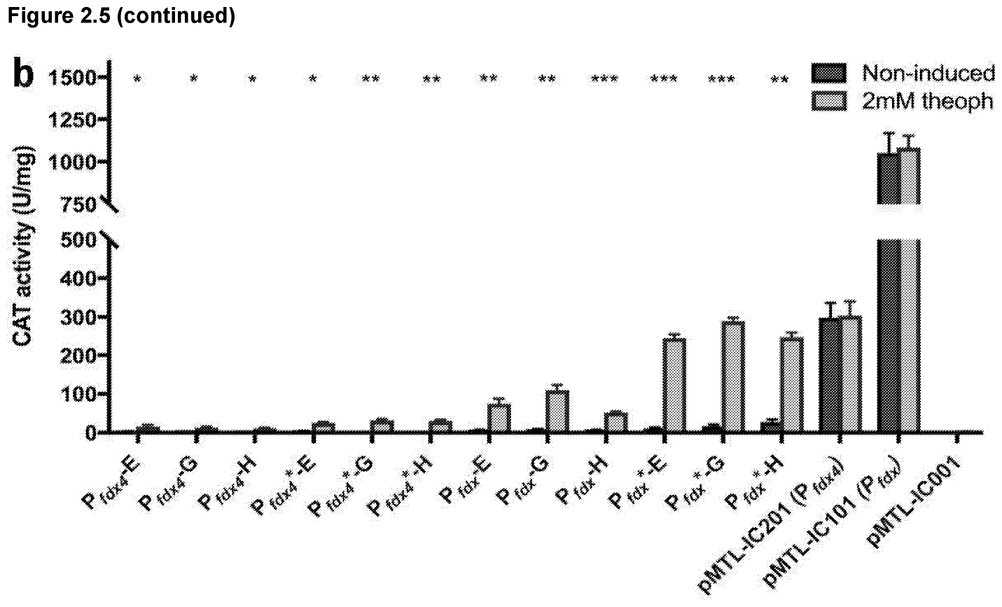

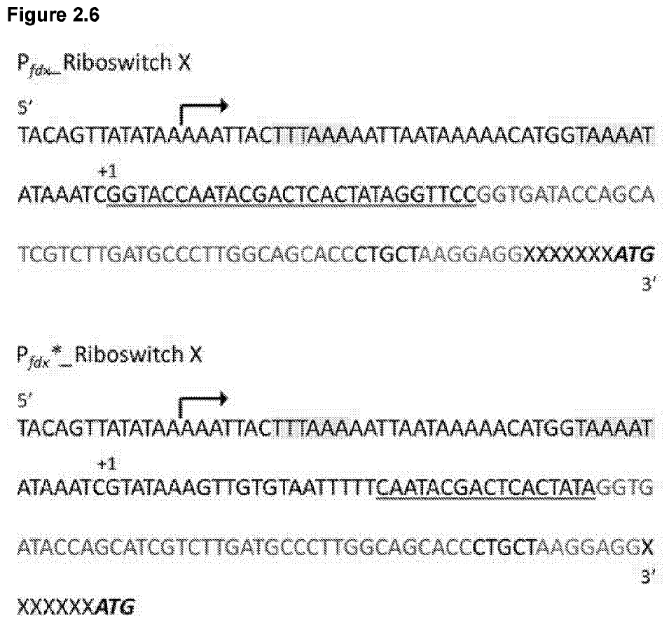

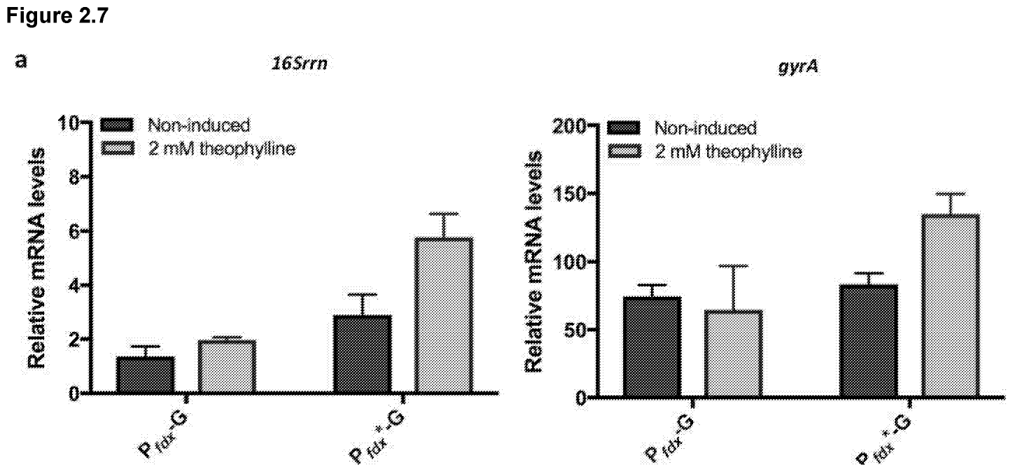

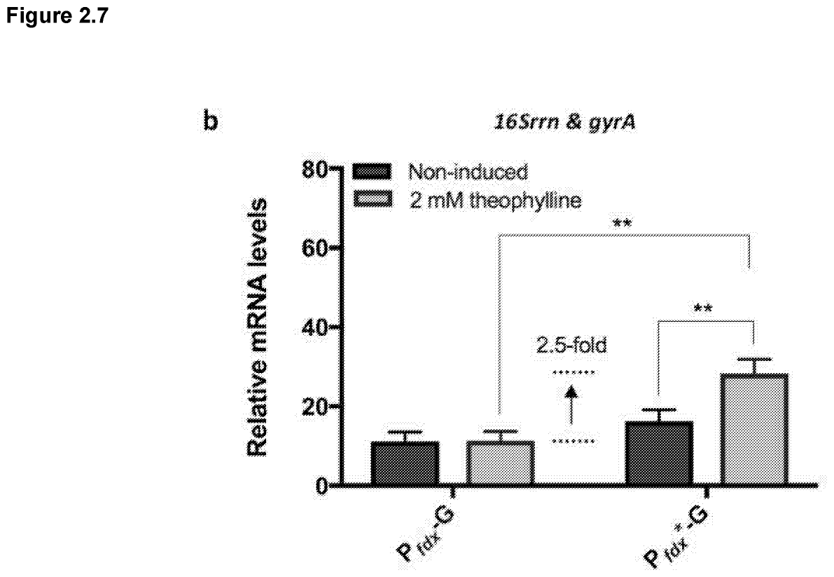

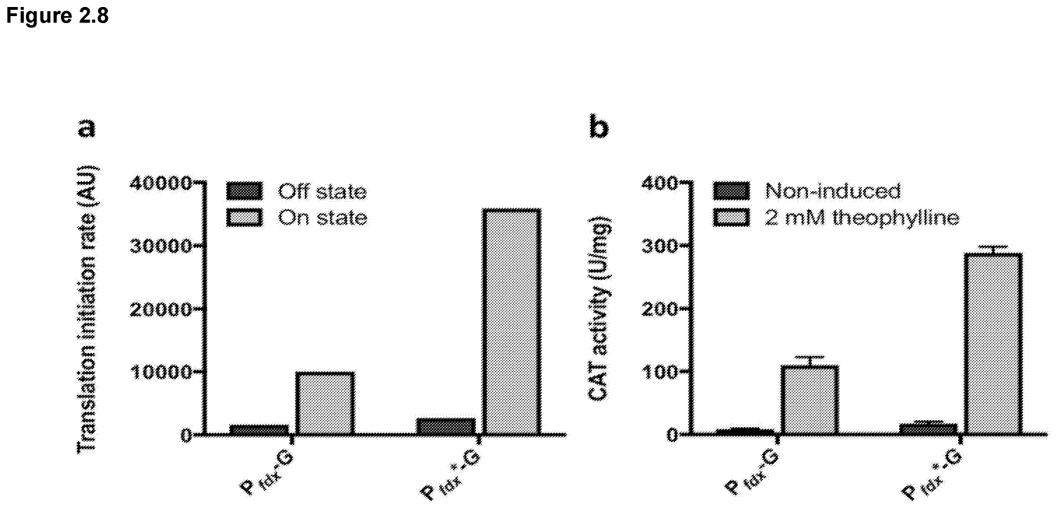

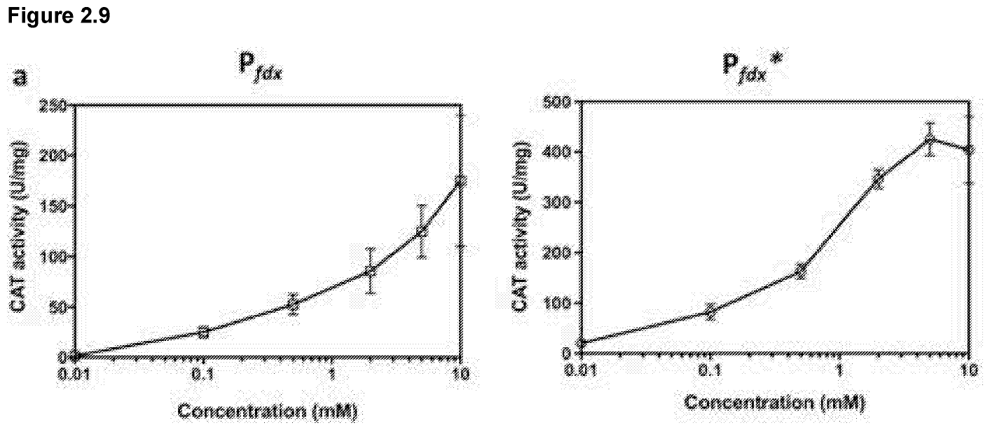

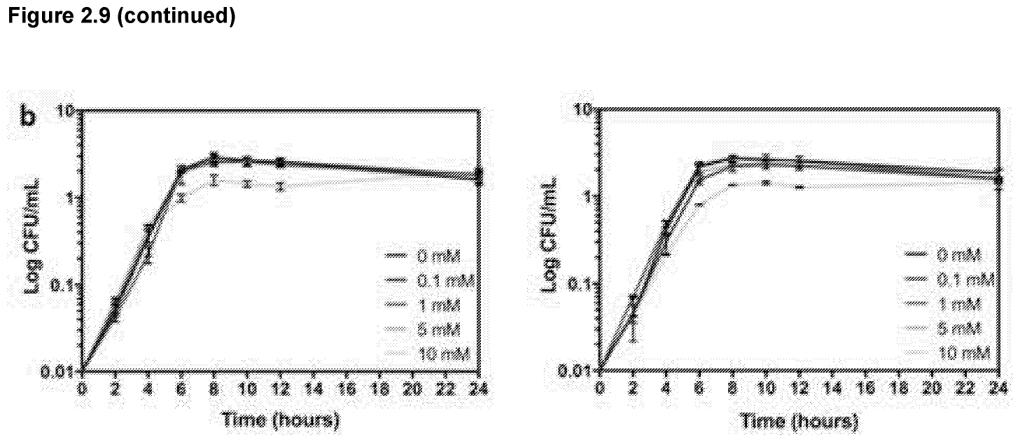

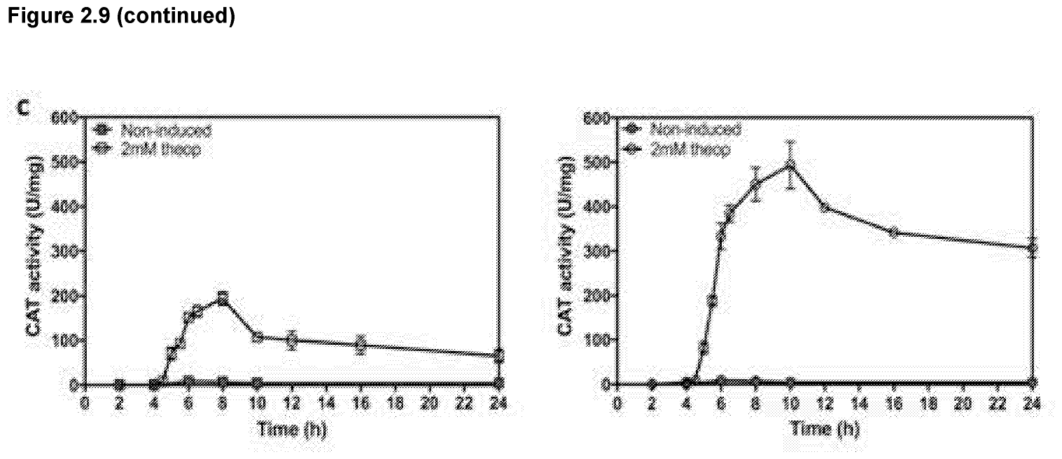

FIG. 2.1 is a schematic illustration of the riboswitch-reporter plasmid. E. coli/Clostridium shuttle plasmid containing the catP reporter under the control of Pfdx. Riboswitches -D to -J were placed downstream of the TSS. (−) is the Gram negative ColE1 origin of replication that allows replication of the shuttle plasmid in E. coli. traJ encodes the TraJ protein, needed for conjugative plasmid transfer. (4) is the Gram-positive replicon derived from the C. botulinum plasmid pBP1;

FIG. 2.2 shows a 5′ RACE experiments on Pfdx. (a) The agarose gel of the PCR amplification products of three independent DNA samples that were reverse transcribed from mRNA template according to the 5′ RACE protocol. cDNA transcription was performed with a primer aligning 250 bp downstream of the translational start site. Extension PCR was performed with a primer aligning to the adaptor at the 3′ Pfdx end of the cDNA (corresponding with the 5′ UTR of the mRNA) and a primer aligning 190 bp downstream of the translational start site, producing a product of at least 0.2 kbp. L is 2-log ladder. (b) Sequence of Pfdx and 5′ RACE sequencing results. The putative −10 and −35 boxes were annotated manually in agreement with the determined TSS (+1). The translational start site is in bold and italicised. The 5′ UTR is underlined;

FIG. 2.3 shows riboswitch library CAT results (a) CAT activity and its ligand dependant induction in each pMTL-111-reporter plasmid. Cells harbouring the different plasmids were grown in TYG medium and induction with 2 mM theophylline took place when they had reached the early exponential growth phase (OD600≈0.5). The reporter plasmids pMTL-IC101 (Pfdx-catP), pMTLIC001 (promoterless catP) as well as the WT strain were used as controls. Error bars represent standard deviations of three biological replicates. Asterisks indicate statistically significant induction values for *p≤0332, **p≤4.0021, ***p≤0.0002, ****p≤0.0001 (paired two-tailed Student's t-test). (b) Activation ratio of riboswitches -D to -J. The activation ratio in each riboswitch-based reporter plasmid was calculated by dividing the value of CAT activity measured in the presence of the inducer by the value of that in the absence of inducer;

FIG. 2.4 shows a schematic representation of a functional model of the theophylline responsive riboswitch. In the absence of theophylline, the RBS is sequestered via a stem-loop formed in the mRNA. After theophylline binding, the riboswitch conformation changes, releasing the RBS and allowing the translation of the gene of interest (catP);

FIG. 2.5 shows CAT results of theophylline responsive riboswitches with different 5′ UTRs (a) The sequences of the constructed theophylline-responsive switches. The putative −35 and −10 sequences are in bold. The TSS is indicated as +1. The 5′ UTR sequences downstream of the TSS are underlined. Core elements replaced to create the synthetic hybrid promoter Pfdx4 are in red. (b) Library of theophylline-dependent riboswitches tested in C. sporogenes in the presence and absence of 2 mM theophylline. Induction took placed at 4 hours, when cells had reached the early exponential growth phase (OD600≈0.5) and CAT activity was determined on cell lysates derived from stationary cultures. The reporter plasmids pMTL-IC201 (Pfdx4-catP), pMTL-IC101 (Pfdx-catP) and pMTL-IC001 (promoterless catP) were used as controls. Error bars represent standard deviations of three biological replicates. Asterisks indicate statistically significant induction values (paired two-tailed Student's t-test);

FIG. 2.6 shows nucleotide sequences of the theophylline responsive riboswitch located downstream of Pfdx and Pfdx*. The putative promoter −35 and −10 sequences are shaded in grey. The TSS is indicated with +1. The linker sequence is underlined. The aptamer sequence and the RBS are highlighted in red and blue respectively. Xs represent riboswitch-specific nucleotides. The translational start site is in bold and italicized. The linker sequences from Pfdx and Pfdx* were obtained from 101 and 185, respectively;

FIG. 2.7 shows RT-qPCR results. catP transcript abundance under the control of either Pfdx-riboswitch-G or Pfdx*-riboswitch-G relative to (a) single reference genes 16Srrn or gyrA; and (b) both reference genes 16Srrn and gyrA. Total RNA was isolated from late exponential cultures grown in the presence and absence of 2 mM theophylline. Error bars represent standard deviations of three biological replicates. Statistically significant differences are indicated with asterisks (paired/unpaired two-tailed Student's t-test);

FIG. 2.8 shows the predicted and observed behaviours for riboswitch-G downstream Pfdx or Pfdx* in C. sporogenes. (a) Predicted riboswitch responses using the Riboswitch Calculator from the Salis Laboratory 180. (b) Observed riboswitch responses (same data as in FIG. 2.6);

FIG. 2.9 shows the dynamic and kinetic profiles of the theophylline responsive riboswitch located downstream of Pfdx or Pfdx*. (a) Response to different concentrations of the inducer theophylline. Cells containing the reporter plasmid with riboswitch G downstream of either Pfdx or Pfdx* were cultivated in TYG medium supplemented with various concentrations of theophylline (0, 0.1, 0.5, 2, 5 and 10 mM) at early exponential growth phase (4 hours of growth, OD600≈0.5); CAT activity was measured in cell lysates derived from stationary phase cultures. (b) Optical density (OD600) of C. sporogenes harbouring the same reporter plasmids in response to 0.1-10 mM of theophylline supplemented at time zero. (c) CAT expression profiles over time. Cells harbouring the reporter plasmids were cultivated in TYG medium in the presence or absence of 2 mM theophylline; CAT was measured on cell lysates from stationary phase cultures. (d) Stability profile of theophylline in C. sporogenes cultures over time. In all cases, error bars represent the standard deviations of three biological replicates;

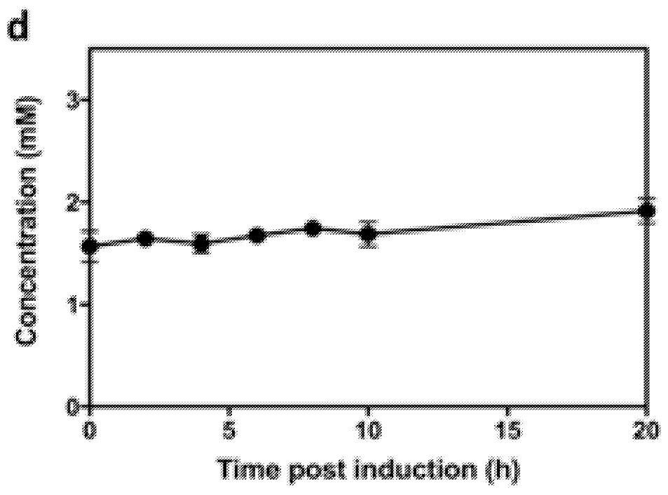

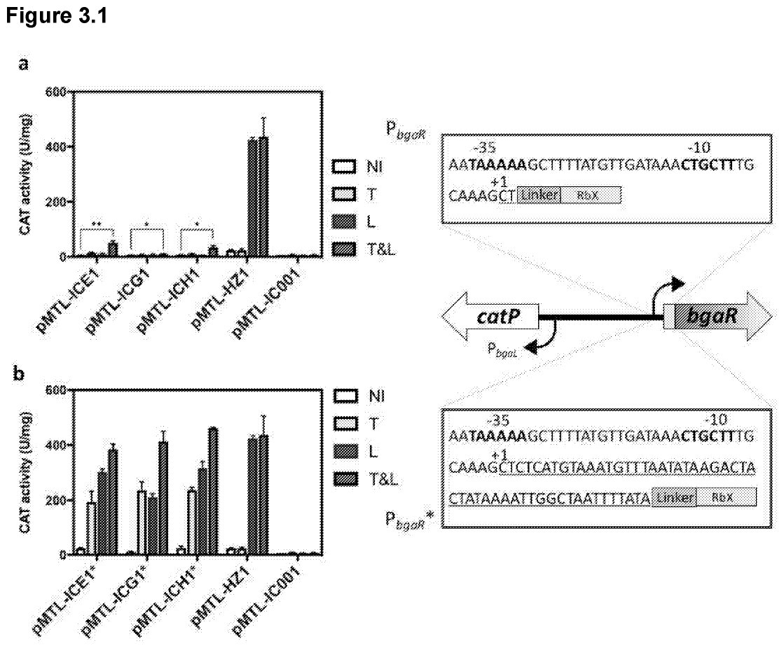

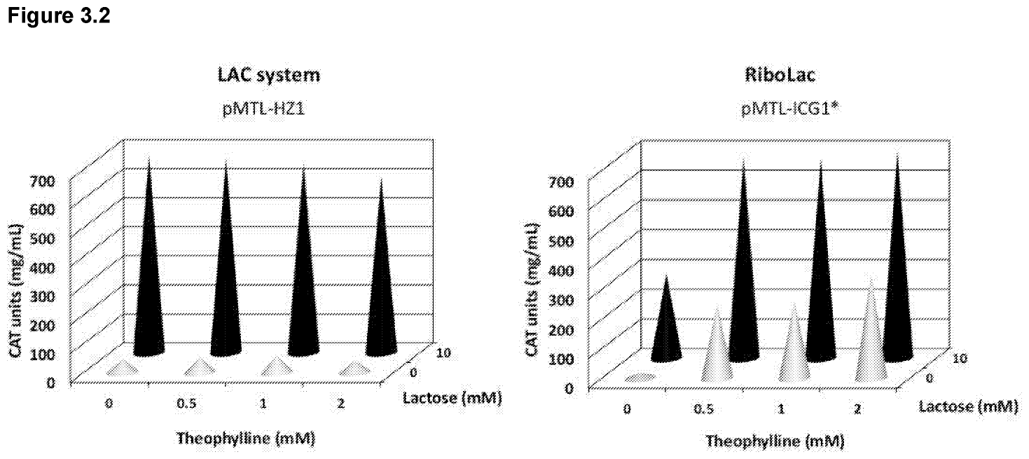

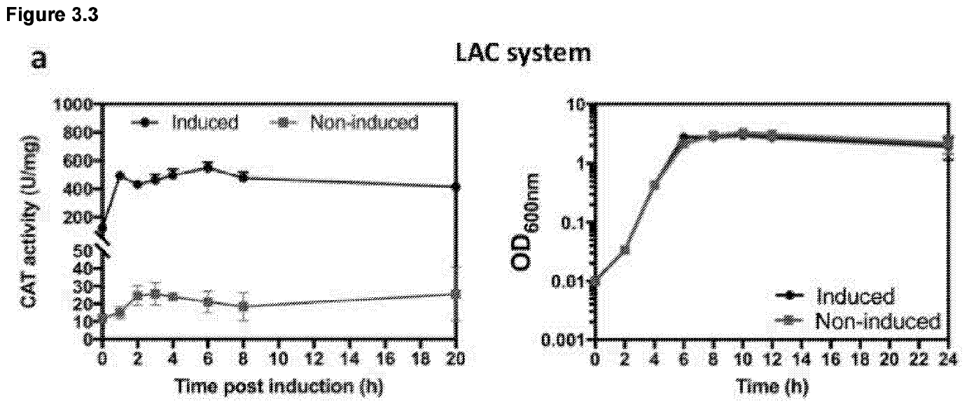

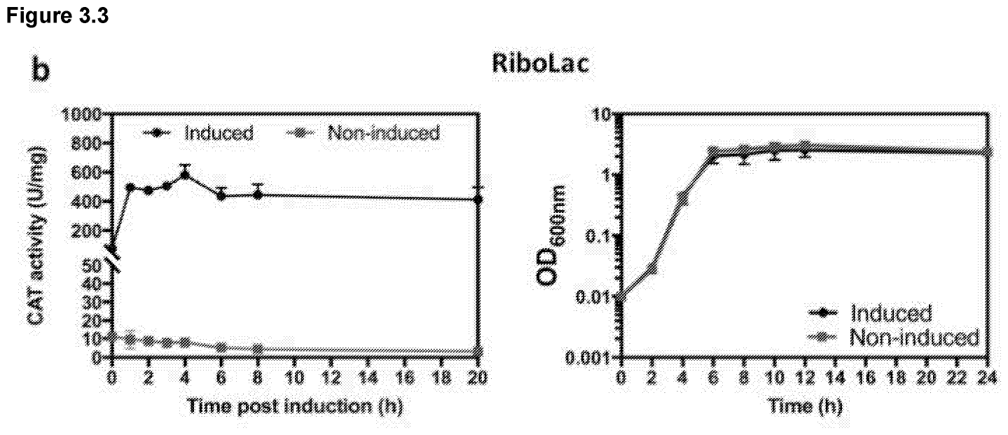

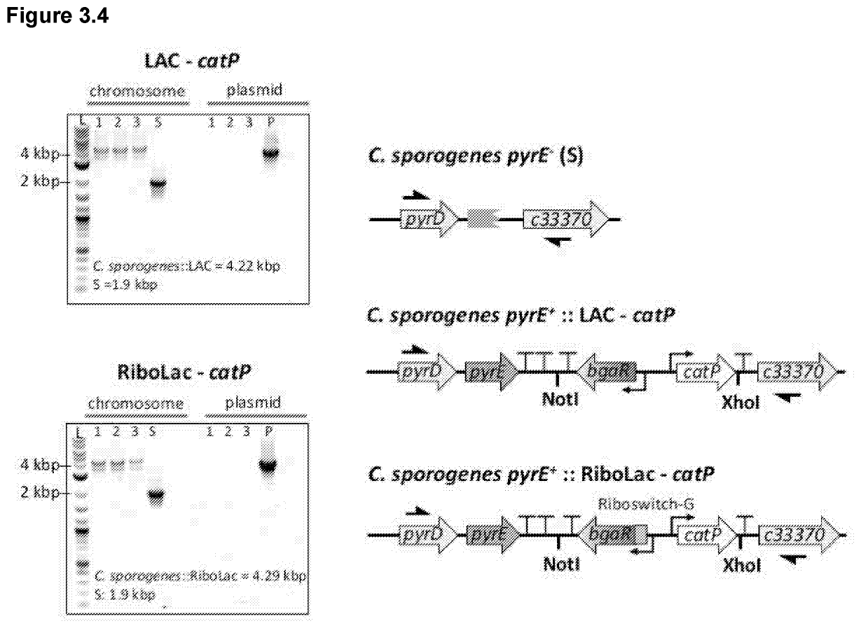

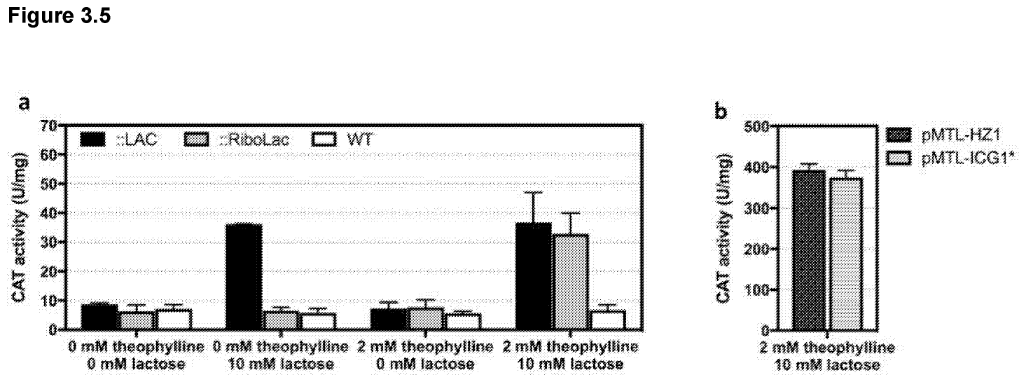

FIG. 3.1 shows the response of the dual theophylline-LAC systems (RiboLacs) in C. sporogenes to different concentrations of theophylline and lactose. (a) CAT results of the dual systems when the riboswitches are placed downstream of PbgaR and nucleotide sequence of PbgaR. (b) CAT results of the dual systems when the riboswitches are placed downstream of PbgaR* and nucleotide sequence of PbgaR*. In both cases, cells harbouring the different plasmids were cultivated in TYG medium supplemented with either 2 mM theophylline (T) or 10 mM lactose (L), with both 2 mM theophylline and 10 mM lactose (T&L) or in the absence of inducers (NI). The vector bearing the LAC system, pMTL-HZ1, and the promoter-less vector, pMTL-IC001, were used as positive and negative controls, respectively. Induction took place at 4 hours, when cells had reached the early exponential growth phase (OD600≈0.5); CAT activity was determined on cell lysates derived from stationary cultures. Error bars represent the standard deviation of three biological replicates. Asterisks indicate statistically significant induction values for *p≤0.0332, **p≤0.0021, ***p≤0.0002, ****p≤0.0001 (paired two-tailed Student's t-test). RbX refers to any of the three riboswitches -E, -G and -H;

FIG. 3.2 shows a dose-response comparison. Response of the RiboLac and LAC system to various concentrations of theophylline in the presence and absence of 10 mM lactose. Cells containing plasmids pMTL-HZ1 and pMTL-ICG1* were cultivated in TYG medium supplemented with various concentrations of the inducers theophylline and lactose when they had reached early exponential growth phase (OD600≈0.5). CAT activity was determined on cell lysates from stationary cultures. Data represent the mean of three biological replicates;

FIG. 3.3 shows time-course induction assays. CAT expression profiles and growth monitored over time of C. sporogenes harbouring (a) the LAC system (pMTLHZ1) and (b) the RiboLac (pMTL-ICG1*) in the presence and absence of inducers. In both cases, cells were cultivated in TYG medium lacking lactose as well as cultures to which 2 mM theophylline and 10 mM lactose were added when they had reached early exponential growth phase (at 4 hours of growth, OD600≈0.5). Samples at 4 hours were collected immediately after induction (time 0 postinduction). CAT activity was determined on cell lysates derived from stationary phase cultures. Error bars represent the standard deviation of three biological replicates;

FIG. 3.4 shows an agarose gel electrophoresis of PCR products of the pyrE locus following integration of the Lac-catP and RiboLac-catP sequences. PCRs were conducted using chromosomal external primers IC001-f and IC002-r. Three separate clones, derived from three independent conjugations, were examined. S lane shows PCR of insertion site in C. sporogenes pyrE- strain using both external primers. Plasmid loss was verified in the same samples by PCR, using screening primers IC007-f and IC008-r, which align to the integration vector. P lane shows PCR of the pMTL-JH29T-L and pMTL-JH29TR plasmids. L is 2-log ladder;

FIG. 3.5 shows a CAT assay of systems integrated into the chromosome. (a) CATassay of C. sporogenes::LAC and C. sporogenes::RiboLac integrants in the presence or absence of the inducers theophylline and lactose. The WT strain was used as negative control. (b) CAT assay of cells harbouring the vectors pMTLHZ1 and pMTL-ICG1*. In both cases, cells were cultivated in TYG medium lacking theophylline and lactose, as well as in the presence of either one of the inducers or in the presence of both inducers. Induction took place when cultures had reached the early exponential growth phase (OD600≈0.5). CAT activity was determined on cell lysates derived from stationary phase cultures. Error bars represent the standard deviation of three biological replicates;

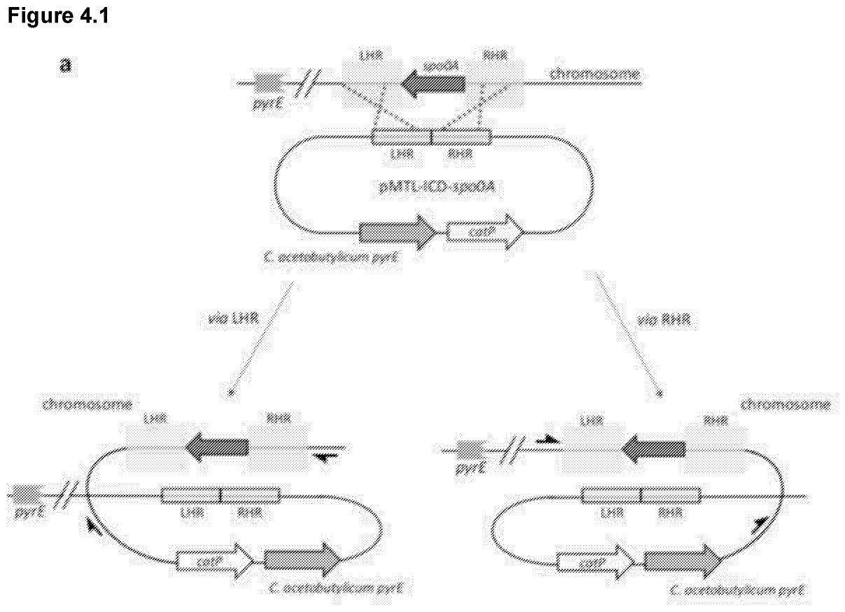

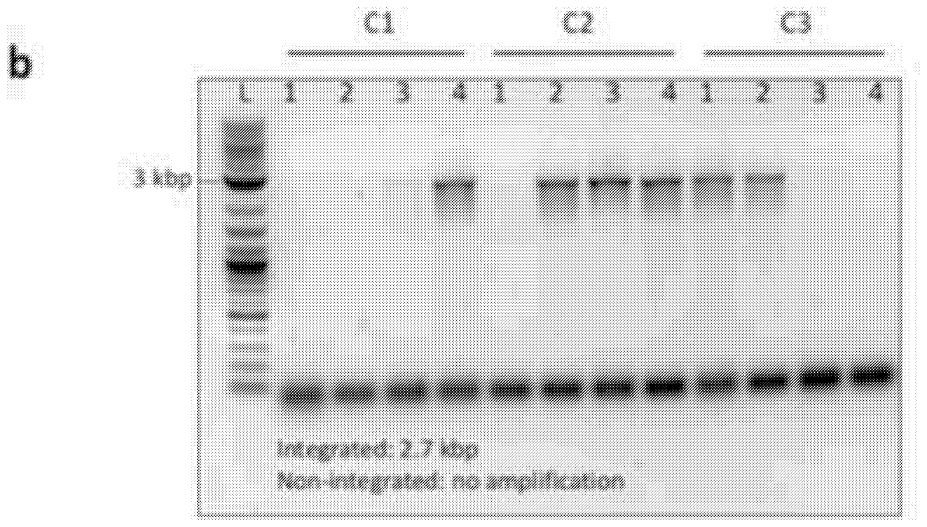

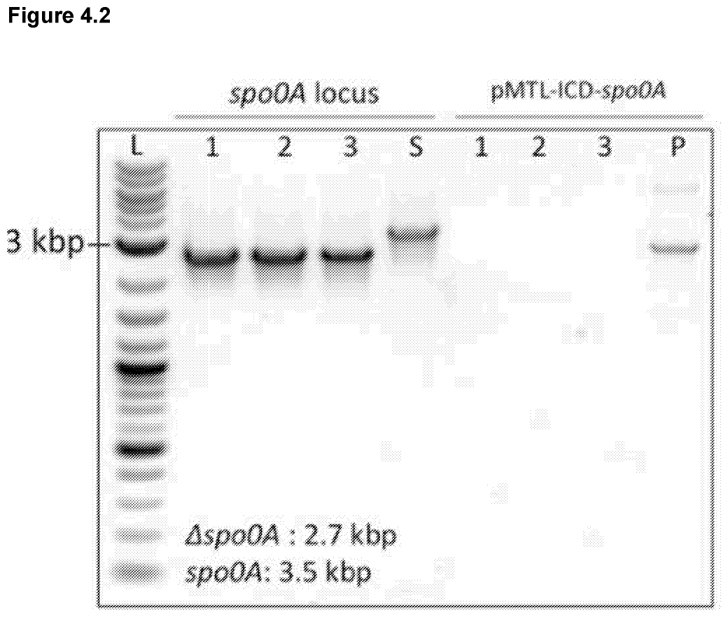

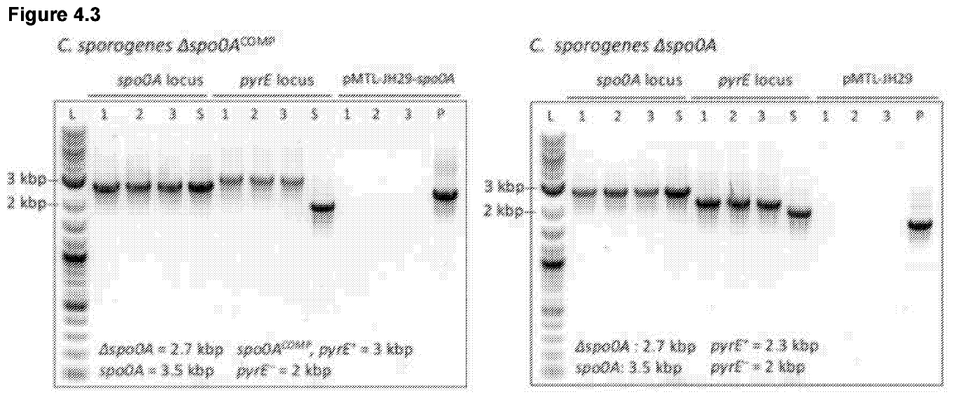

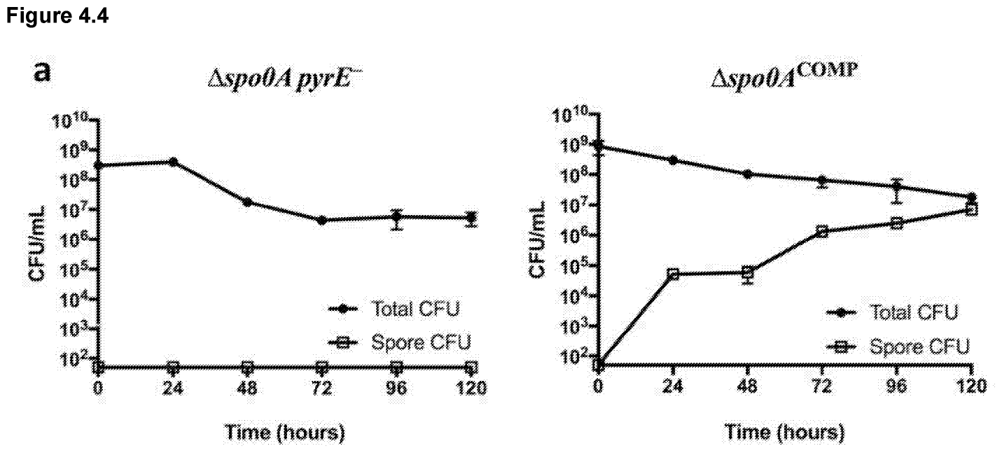

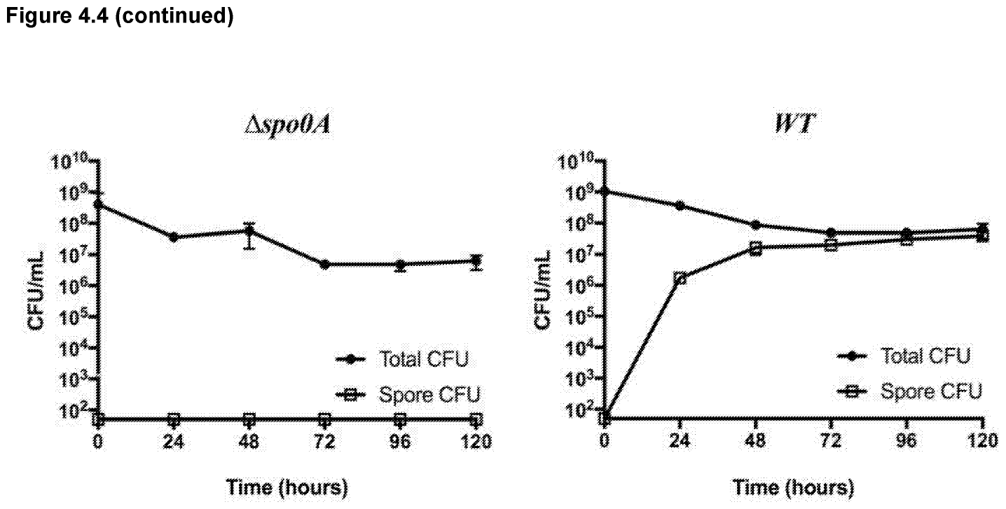

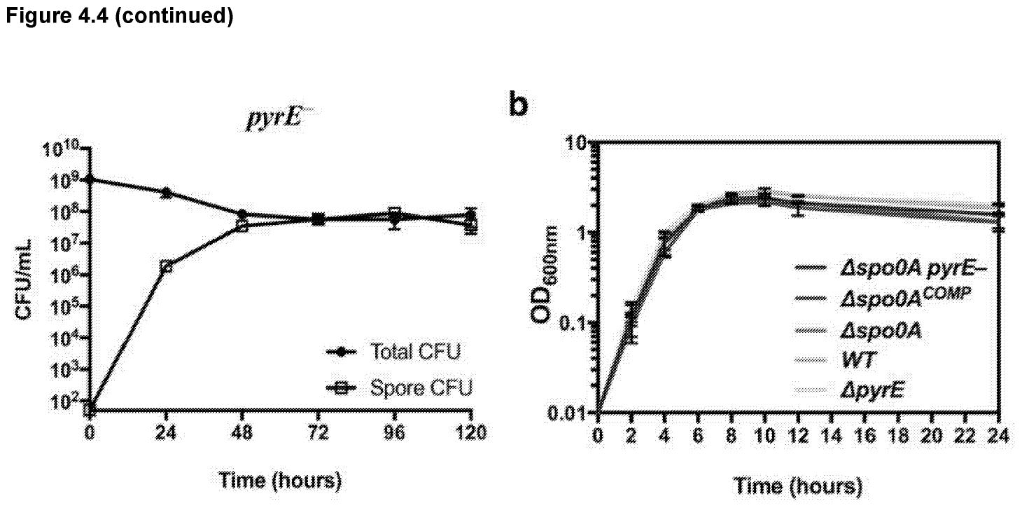

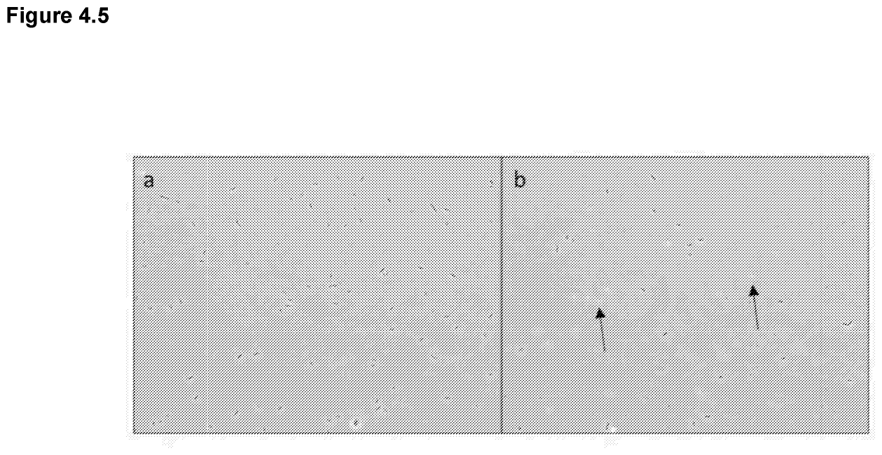

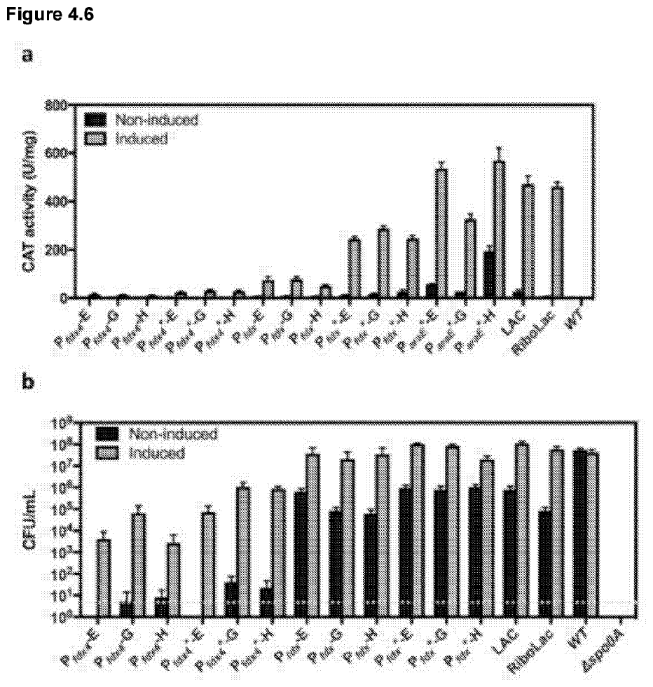

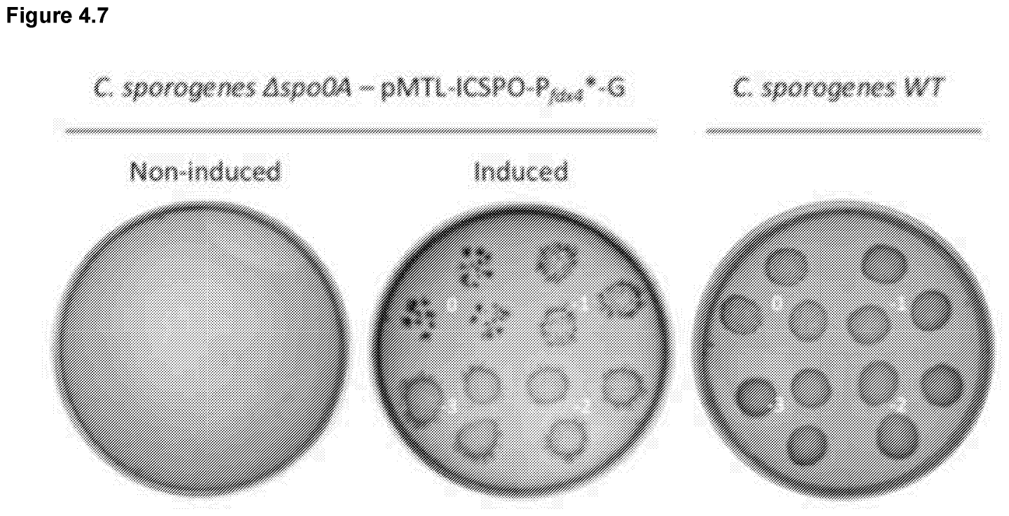

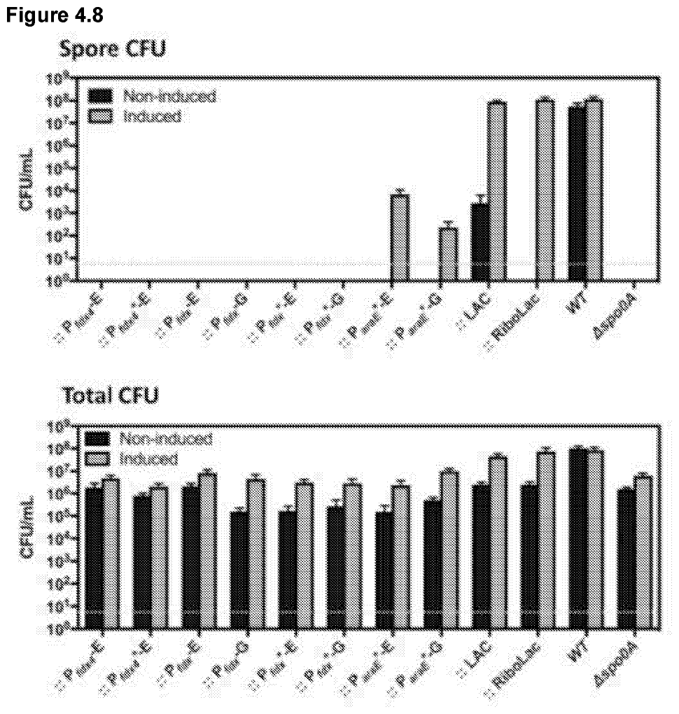

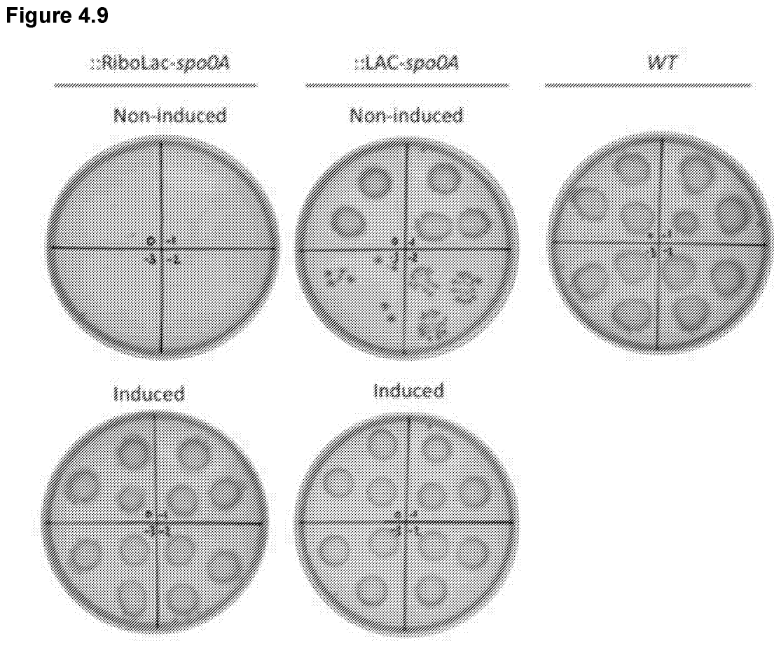

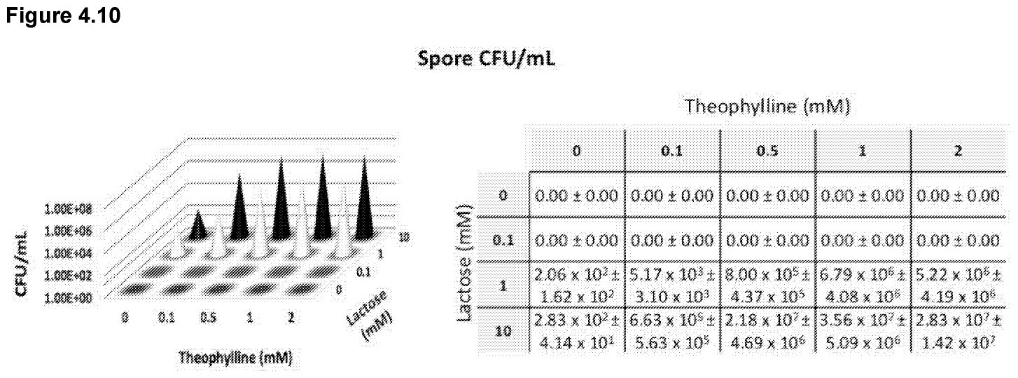

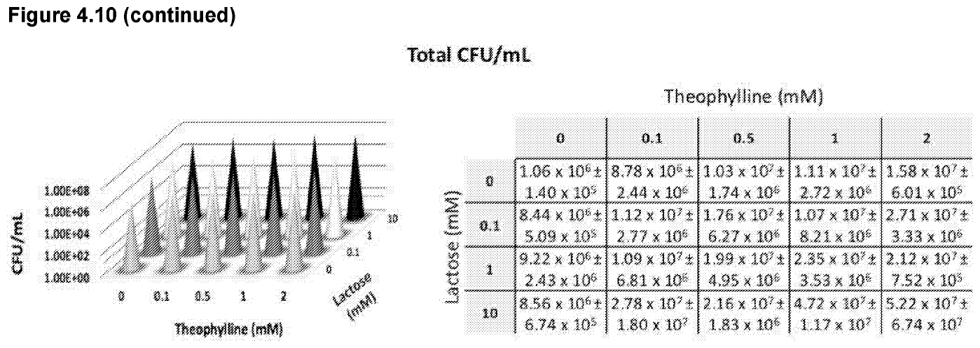

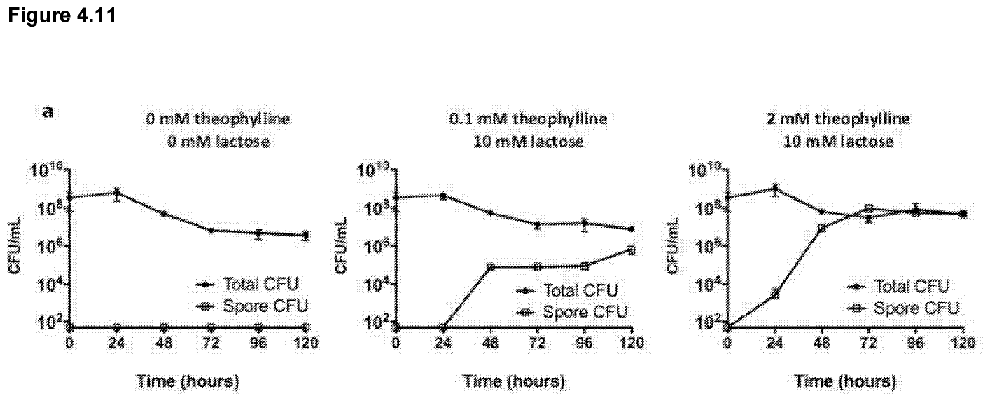

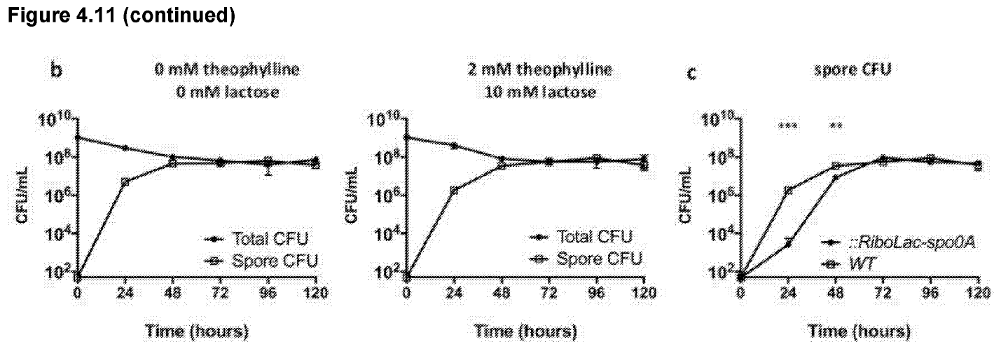

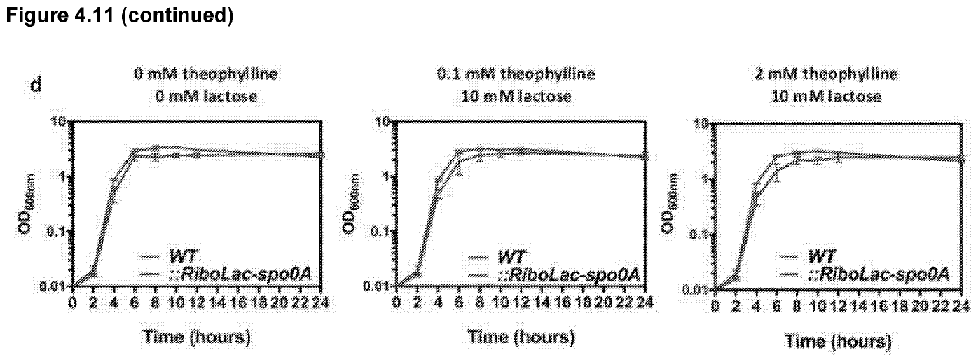

FIG. 4.1 shows an allelic exchange single crossovers (a) Schematic illustration of single-cross events via left (LHR) and right homologous regions (RHR). (b) Agarose gel electrophoresis of PCR products of single-crossover spo0A mutants. Colonies from three independent conjugations (C1, C2 and C3) were analysed by colony PCR using the pairs of primers IC003-f/IC006-r and IC005-f/IC004-r, to detect plasmid integration within the chromosome of C. sporogenes through the LHR and RHR, respectively. The pairs of primers are specific combinations of one primer aligning to the chromosome and a second primer aligning to the deletion vector (see Appendix, Fig S4). L is 2-log ladder. The gel only shows PCR products from amplifications via the LHR, since no plasmid integrations were detected through the RHR;

FIG. 4.2 shows an allelic exchange double crossovers. Agarose gel electrophoresis of PCR products of the spo0A locus following deletion of the spo0A gene from the C. sporogenes pyrE-strain. PCRs were conducted using chromosomal external primers IC003-f and IC004-r. Three separate clones, derived from three independent single-cross mutants were examined. Loss of the plasmid was verified in the same samples by colony PCR, using screening primers IC005-f/IC006-r. L is 2-log ladder. S is C. sporogenes pyrE- strain. P lane shows PCR of the pMTL-ICD-spo0A plasmid;

FIG. 4.3 shows ACE double crossovers. Agarose gel electrophoresis of PCR products of the spo0A locus, the insertion site in the pyrE locus and the integration plasmids pMTL-JH29-spo0A and pMTL-JH29 for spo0A complementation and repair of pyrE, respectively. Three independent clones, derived from three independent conjugations, were examined. PCRs were conducted using chromosomal external primers IC003-f/IC004-r, IC001-f/IC002-r for and IC007-f/IC008-r for the spo0A locus, the pyrE locus and the integration plasmids, respectively. PCR products were further verified by DNA sequencing. L is 2-log ladder; S is C. sporogenes pyrE- parent strain; P is pMTL-JH29-spo0A or pMTLJH29 vectors for spo0A complementation and pyrE repair, respectively;

FIG. 4.4 shows (a) Sporulation and (b) growth profiles of C. sporogenes Δspo0A pyrE-, C. sporogenes Δspo0ACOMP, C. sporogenes Δspo0A, C. sporogenes WT and C. sporogenes pyrE. After culture synchronization, samples were removed at the indicated times points, serially diluted in and spotted onto TYG agar. For spore counting, samples were heat-treated for 20 minutes at 80° C. prior to serial dilution. The optical density of the cultures (OD600) was monitored over the first 24 hours of growth. Error bars represent the standard deviation of three biological replicates from two independent assays. Y axis set at the limit of detection (50 spores/mL);

FIG. 4.5 shows a microscopic view of (a) C. sporogenes Δspo0A and (b) C. sporogenes WT cultures after 120 hours. Spores appear phase bright (arrows) and vegetative cells and debris as purple phase dark elements;

FIG. 4.6 shows an assay of plasmid-based conditionally sporulating strains (a) Plasmid based CAT assays of C. sporogenes strains expressing catP under the control of various inducible systems generated in this study. Cell cultivations and assay were performed. Error bars represent the standard deviation of three biological replicates. (b) Sporulation profile of C. sporogenes Δspo0A strains harbouring the different pMTL-ICSPO plasmids. Cultures were grown synchronized and used to inoculate the starting culture of the assay. Samples were removed at time 0 to verify the lack of spores in the starting cultures (data not shown). At early exponential phase (OD600≈0.5) cultures were split into two equal volumes and the following inducers added to one of them: 2 mM theophylline (if regulated by riboswitches), 10 mM lactose (if regulated by the LAC system) and 2 mM theophylline and 10 mM lactose (if regulated by the RiboLac). After 120 hours, cell suspensions were heat-treated, serially diluted and spotted onto TYG agar. Colonies were enumerated after 48 hours of incubation under anaerobic conditions. Error bars represent the standard deviation of three biological replicates from two independent assays. Dotted line represents the limit of detection (50 spores/mL);

FIG. 4.7 shows plates of heat-treated plasmid-based conditionally sporulating strain. Agar plates of serially diluted heat-treated induced and non-induced cultures of the plasmid-based conditionally sporulating strain C. sporogenes harbouring the spo0A expressing plasmid with spo0A driven by Pfdx4*-G and C. sporogenes WT. Numbers indicate 10× dilution. Plates were examined after 48 hours of incubation under anaerobic conditions;

FIG. 4.8 shows the sporulation capacity of stable conditionally sporulating strains. Error bars represent the standard deviation of three biological replicates from two independent assays. Dotted line represents the limit of detection (50 spores/mL);

FIG. 4.9 shows plates of heat-treated stable conditionally sporulating strains. Agar plates of serially diluted heat-treated cultures of C. sporogenes::RiboLac-spo0A, C. sporogenes::LAC-spo0A and C. sporogenes WT in the presence and absence of induction. Numbers indicate 10× dilutions. Plates were examined after 48 hours of incubation under anaerobic conditions;

FIG. 4.10 shows a dose-dependent increase in sporulation efficiency in C. sporogenes::RiboLac spo0A, represented as expression matrices and numerically. Starting cultures were split into 20 equal volumes and each of them cultured under different inducer concentrations; total and heat-resistant CFU were enumerated after 120 hours of growth. The mean values and standard deviations represent the CFUs/mL of biological triplicates in two independent assays. Dotted line in the matrices represents the limit of detection (50 spores/mL); and

FIG. 4.11 shows the sporulation profiles of (a) C. sporogenes::RiboLac-spo0A and (b) C. sporogenes WT with various concentrations of inducers. (c) Comparison of the sporulation profiles of both strains. Asterisks indicate statistically significant induction values for *p≤0.0332, **p≤0.0021, ***p≤4.0002, ****p≤4.0001 (unpaired two-tailed Student's t-test). Y axis set at the limit of detection (50 spores/mL). (d) Optical density (OD600) of C. sporogenes::RiboLac-spo0A and C. sporogenes WT in response to various concentrations of theophylline and lactose. Error bars represent the standard deviation of three biological replicates from two independent assays.

### EXAMPLES

### Materials and Methods

### Strains, Media and Growth Conditions.

All the E. coli and Clostridium strains used in this application are listed in Table 2. E. coli TOP C (Invitrogen) was used as a general host for plasmid construction and propagation. E. coli CA434 was used as the donor strain for conjugation. All E. coli strains were transformed through electroporation using a MicroPulser™ system (BioRad). E. coli strains were grown at 30 or 37° C., in Luria-Bertani (LB) medium supplemented with chloramphenicol (25 μg/mL in solid and 12.5 μg/mL in liquid media), erythromycin (500 μg/mL) or kanamycin (50 μg/mL) when necessary. Media for clostridial strains were supplemented with the following antibiotic/inducer/supplement when appropriate: thiamphenicol (15 μg/mL), erythromycin (10 μg/mL), D-cycloserine (500 μg/mL), theophylline (0.1-10 mM), glucose 0.05% w/v. Clostridium strains were grown at 37° C. in an anaerobic cabinet (MG1000 anaerobic workstation; Don Whitley Scientific Ltd).

TABLE 2

| Strain | Source^{b} |
|---|---|
| C. sporogenes NCIMB 10696 | CRG |
| C. sporogenes pyrE⁻ | [*] |
| C. sporogenes::LAC | This application |
| C. sporogenes::RiboLac | This application |
| C. sporogenes pyrE⁻ Δspo0A | This application |
| C. sporogenes Δspo0A | This application |
| C. sporogenes Δspo0A^{COMP} | This application |
| C. sporogenes Δspo0A::P_{fdx4}-E-spo0A | This application |
| C. sporogenes Δspo0A::P_{fdx4}*-E-spo0A | This application |
| C. sporogenes Δspo0A::P_{fdx}-E-spo0A | This application |
| C. sporogenes Δspo0A::P_{fdx}-G-spo0A | This application |
| C. sporogenes Δspo0A::P_{fdx}*-E-spo0A | This application |
| C. sporogenes Δspo0A::P_{fdx}*-G-spo0A | This application |
| C. sporogenes Δspo0A::P_{araE}*-E-spo0A | This application |
| C. sporogenes Δspo0A::P_{araE}*-G-spo0A | This application |
| C. sporogenes Δspo0A::LAC-spo0A | This application |
| C. sporogenes Δspo0A::RiboLac-spo0A | This application |

*Heap, J. T. et al. Integration of DNA into bacterial chromosomes from plasmids without a counter-selection marker. Nucleic Acids Res. 40, e59 (2012)

### Reagents.

All PCR reactions were performed using KOD-Hot Start Polymerase 2× Master Mix (Merck Millipore) or DreamTaq Green PCR Master Mix (Thermo Fisher Scientific). T4 ligase (Promega) was used for DNA ligation reactions. Restriction enzymes were purchased from New England Biolabs. Theophylline was purchased from Sigma-Aldrich.

### Plasmid Design, Construction and Transformation.

Oligonucleotide primers were synthesized by Sigma-Aldrich and are listed in Table 4. Plasmids were constructed by restriction enzyme-based cloning procedures. Constructs were verified by DNA sequencing (Eurofins). All the plasmids used in this study are listed in Table 3. Details of plasmid construction are given in the Supporting Information. Protospacer sequences were designed according to the protocol described at http://benchling.com/pub/ellis-crspr-tools.

TABLE 3

| Plasmid | Reference source |
|---|---|
| pMTL82251 | [*] |
| pMTL83151 | [*] |
| pMTL-HZ1 | Wang, H. CRG |
| pMTL-IC001 | This application |
| pMTL-IC1201 | This application |
| pMTL-ICMO1 | This application |
| pMTL-ICMO2 | This application |
| pMTL-ICMO1O2 | This application |
| pMTL-ICMB | This application |
| pMTL-ICT1 | This application |
| pMTL-ICT2 | This application |
| pMTL-ICT3 | This application |
| pMTL-ICT4 | This application |
| pMTL-ICT5 | This application |
| pMTL-ICT6 | This application |
| pMTL-ICT7 | This application |
| pMTL-ICT8 | This application |
| pMTL-ICT9 | This application |
| pMTL-ICT10 | This application |
| pMTL-ICT11 | This application |
| pMTL-ICM1 | This application |
| pMTL-ICM2 | This application |
| pMTL-ICM3 | This application |
| pMTL-ICM4 | This application |
| pMTL-ICM5 | This application |
| pMTL-ICM6 | This application |
| pMTL-ICM7 | This application |
| pMTL-ICM8 | This application |
| pMTL-ICM9 | This application |
| pMTL-ICM10 | This application |
| pMTL-ICM11 | This application |
| pMTL-IC101 | This application |
| pMTL-IC111-D | This application |
| pMTL-IC111-E | This application |
| pMTL-IC111-E* | This application |
| pMTL-IC111-F | This application |
| pMTL-IC111-G | This application |
| pMTL-IC111-H | This application |
| pMTL-IC111-I | This application |
| pMTL-IC111-J | This application |
| pMTL-IC121-E | This application |
| pMTL-IC121-G | This application |
| pMTL-IC121-H | This application |
| pMTL-IC201 | This application |
| pMTL-IC201-E | This application |
| pMTL-IC201-G | This application |
| pMTL-IC201-H | This application |
| pMTL-IC221-E | This application |
| pMTL-IC221-G | This application |
| pMTL-IC221-H | This application |
| pMTL-ICE1 | This application |
| pMTL-ICG1 | This application |
| pMTL-ICH1 | This application |
| pMTL-ICE1* | This application |
| pMTL-ICG1* | This application |
| pMTL-ICH1* | This application |
| pMTL-JH29 | [**] |
| pMTL-JH29T | This application |
| pMTL-JH29T-L | This application |
| pMTL-JH29T-R | This application |
| pMTL-ICLG1* | This application |
| pMTL-ICLH1* | This application |

*Heap, J. T., Pennington, O. J., Cartman, S. T. & Minton, N. P. A modular system for Clostridium shuttle plasmids. J. Microbiol. Methods 78, 79-85 (2009).
**Heap, J. T. et al. Integration of DNA into bacterial chromosomes from plasmids without a counter-selection marker. Nucleic Acids Res. 40, e59 (2012)

TABLE 4

| SEQ ID No. | Primer | Sequence |
|---|---|---|
| 14 | IC103-f | TATAGGTCTCAATGGTATTTGAAAAAAT |
| 15 | IC104-r | TATACTCGAGTTAACTATTTATCAATTCCTG |
| 16 | IC105-f | CAGCGGCCGCATGGTATTTGAAAAAATTGATAAAAA |
| 18 | IC301-f | TATAGCGGCCGCAAATATTCTTATATTTAGA |
| 19 | IC302-r | TATAGGTCTCACCATTTCTACCTCCTA |
| 20 | IC303-f | AAAGCAGTTTATTTAAAATAAATTCTTCTAAATATAAGAATATTTTAAAGAAATATCTTT |
| 21 | IC304-r | TATTTTAAATAAACTGCTTTGCAAAGCTCTCATGT |
| 22 | IC305-f | TTATAAGATTATATAGAACTTTTTAACTATTCTAAAAGATTAATTTACATA |
| 23 | IC306-r | AAAAGTTCTATATAATCTTATAAATTTTAATAACTAATATATAAAGATA |
| 24 | IC307-f | CACAGCGGCCGCTTrTACCTCCTAACCTATAAAATTAGCC |
| 25 | IC308-r | CACACATATGACCCTCCCAATACATTTAAAATAATTATGTATTCATGAAA |
| 26 | IC309-f | GGCCGGTCTCTTAATAACTAATATATAAAGATATTTCTTTAAAATATTCTTATATTTAGA |
| 27 | IC310-r | CCGCGGCCGCTAATTTAGATATTAATTCTAAATTAAGTGAAATTAATATAGTAATTAT |
| 28 | IC311-f | GGCCGGCCGGTCTCTATTATATAAGATTATAAGAAACATTATAACATATTTTAG |
| 29 | IC312-f | GGCCGGCCGGTCTCTATTAGATTATAAGAAACATTATAACATATTTTAGAACTT |
| 30 | IC313-f | GGCCGGCCGGTCTCTATTATAAGAAACATTATAACATATTTTAGAACTTTTTAA |
| 31 | IC314-f | GGCCGGCCGGTCTCTATTAAACATTATAACATATTTTAGAACTTTTTAACTATT |
| 32 | IC315-f | GGCCGGCCGGTCTCTATTATATAACATATTTTAGAACTTTTTAACTATTCTAAA |
| 33 | IC316-f | GGCCGGCCGGTCTCTATTAACATATTTTAGAACTTTTTAACTATTCTAAAAGATT |
| 34 | IC317-f | GGCCGGCCGGTCTCTATTATTTTAGAACTTTTTAACTATTCTAAAAGATTAATTT |
| 35 | IC318-f | GGCCGGCCGGTCTCTATTAGAACTTTTTAACTATTCTAAAAGATTAATTTACATA |
| 36 | IC319-f | GGCCGGCCGGTCTCTATTATTTTAACTATTCTAAAAGATTAATTTACATATTAAC |
| 37 | IC320-f | GGCCGGCCGGTCTCTATTAACTATTCTAAAAGATTAATTTACATATTAACATTTA |
| 38 | IC321-f | GGCCGGCCGGTCTCTATTATCTAAAAGATTAATTTACATATTAACATTTAATTAT |
| 39 | IC322-f | GGCCGGCCGGTCTCTATTATTTAATATAAGATTATAAGAAACATTATAACATATTTTAGAAC |
| 40 | IC323-f | GGCCGGCCGGTCTCTATTAAAATTATATTGATTATAAGAAACATTATAACATATTTTAGAACTTTTT |
| 41 | IC324-f | GGCCGGCCGGTCTCTATTAAAATTTATAACTAATTAAGAAACATTATAACATATTTTAGAACTTTTTACTATT |
| 42 | IC325-f | GGCCGGCCGGTCTCTATTAAAATTTATAAGATTAATTCTAACATTATAACATATTTTAGAACTTTTTAACTATT |
| 43 | IC326-f | GGCCGGCCGGTCTCTATTAAAATTTATAAGATTATAAGATTGTATATAACATATTTTAGAACTTTTTAACTATT |
| 44 | IC327-f | GGCCGGCCGGTCTCTATTAAAATTTATAAGATTATAAGAAACATATATTCATATTTTAGAACTTTTTAACTATT |
| 45 | IC328-f | GGCCGGCCGGTCTCTATTAAAATTTATAAGATTATAAGAAACATTATAAGTATATTTAGAACTTTTTAACTATT |
| 46 | IC329-f | GGCCGGCCGGTCTCTATTAAAATTTATAAGATTATAAGAAACATTATAACATATAAATCAACTTTTTAACTATT |
| | | CTAAAAG |
| 47 | IC330-f | GGCCGGCCGGTCTCTATTAAAATTTATAAGATTATAAGAAACATTATAACATATTTTAGTTGAATTTAACTATT |
| | | CTAAAAGATTAATT |
| 48 | IC331-f | GGCCGGCCGGTCTCTATTAAAATTTATAAGATTATAAGAAACATTATAACATATTTTAGAACTTAAATTCTATT |
| | | CTAAAAGATTAATTTACA |
| 49 | IC332-f | GGCCGGCCGGTCTCTATTAAAATTTATAAGATTATAAGAAACATTATAACATATTTTAGAACTTTTTAAGATAA |
| | | CTAAAAGATTAATTTACA |
| 50 | IC011-r | TTATATACTTGGTTTATTTACTTGATTATTTCTGTAA |
| 51 | IC012-f | TATTGTATAGCTTGGTATCATCT |
| 52 | IC101-f | ATATGCGGCCGCGTGTAGTAGCCTGTGAAATAAGTAA |
| 53 | IC102-r | ATATGGTGTCACCATTAACACACCTCCTTAAAAATTACAC |
| 54 | IC103-f | TATAGGTCTCAATGGTATTTGAAAAAAT |
| 55 | IC104-r | TATACTCGAGTTAACTATTTATCAATTCCTG |
| 56 | IC105-f | CAGCGGCCGCATGGTATTTGAAAAAATTGATAAAAA |
| 57 | IC106-r | GGCGGTACCGATTTATATTTTACCATGTTTTTATTAATTT |
| 58 | IC107-f | CCCGGTACCAATACGACTCACTATAGGTTCCGGTGATACCAGCATCGTCTTGATGCCCTTG |
| 59 | IC108-r | CCGGTCTCACCATGTTGTTGCCTCCTTAGCAGGGTGCTGCCAAGGGCATCAAGACGATGCT |
| 60 | IC109-r | CCGGTCTCACCATGTTGTTGCCTCCTTAGCAGGGTGCTGCCAAGGGCATCAAGACGATGCT |
| 61 | IC110-r | CCGGTCTCACCATCTTGTTGTTACCTCCTTAGCAGGGTGCTGCCAAGGGCATCAAGACGATGC |
| 62 | IC111-r | CCGGTCTCACCATGTTGTTACCTCCTTAGCAGGGTGCTGCCAAGGGCATCAAGACGATG |
| 63 | IC112-r | CCGGTCTCACCATTAAGTTACCTCCTTAGCAGGGTGCTGCCAAGGGCATCAAGACGATGC |
| 64 | IC113-r | CCGGTCTCACCATTAACACACCTCCTTAGCAGGGTGCTGCCAAGGGCATCAAGACGATGC |
| 65 | IC114-r | CCGGTCTCACCATCTTGTTGACCTCCTTAGCAGGGTGCTGCCAAGGGCATCAAGACGATGC |
| 66 | IC115-r | CCGGTCTCACCATTGGTCGTACACTCCCTTAGCAGGGTGCTGCCAAGGGCATCAAGACGATGC |
| 67 | IC116-r | CCCGGTCTCTATTGAAAAATTACACAACTTTATACGATTTAT |
| 68 | IC117-f | GGTCTCACAATACGACTCACTATAGGTGATACCAGCATCGTCTTGATGCCCTTG |
| 69 | IC118-f | AAACTTAACTTCATGTTAAAAACTTGTTAAAATATAAATCGTATAAAGTTGTGTAATTT |
| 70 | IC119-r | ATTTATATTTTAACAAGTTTTTAACATGAAGTTAAGTTTTTTTTATATAACTGTATAACC |
| 71 | IC263-r | AAATGTGAAATCCGTCACATACT |
| 72 | IC264-r | GGTATCATCTCATCATATATCCCCAATTCACCATCTT |
| 73 | IC265-r | TATAGGCGCGCCTTTACAATCATTGCAATATAATAAAG |
| 74 | M13-f | TGTAAAACGACGGCCAGT |
| 75 | M13-r | CAGGAAACAGCTATGACC |
| 76 | IC333-r | CCGGTCTCAATGCAAATATTGTGGAAAAAGTATGTTAAAGA |
| 77 | IC334-r | CCGGTCTCTGCATCTTGTTGTTACCTCCTTAGCAGGGTGCTGCCAAGGGCATCAAGACGATGC |
| 78 | IC335-r | CCGGTCTCTGCATTAAGTTACCTCCTTAGCAGGGTGCTGCCAAGGGCATCAAGACGATGC |
| 79 | IC336-r | CCGGTCTCTGCATTAACACACCTCCTTAGCAGGGTGCTGCCAAGGGCATCAAGACGATGC |
| 80 | IC337-f | GGCGGTACCGAGCTTTGCAAAGCAGTTTATCAACATAAA |
| 81 | IC338-f | GGCGGTACCTATAAAATTAGCCAATTTTATAGTAGTCTT |
| 82 | IC339-f | GCCCCCTGCAGGAGTAATATAAATGTTATAGATATATTAA |
| 83 | IC340-r | CCGGTCTCAAACCTTATTTTTGTTCTCTACTACCTGGTTTTACAAA |
| 84 | IC341-f | CCCGGGTCTCAGGTTTCCCGACTGGAAAGCGGGCAGTGACCAGGCATCAAATAAAACGAAAGGCTCAGTCGAAA |
| | | GACTGGGCCTTTCGTTTTATCTGTTGTTTGTCGGTGAACGCTCTCT |
| 85 | IC342-r | CCCGCGGCCGCAGTTAATTATTGATTATAAACGCAGAAAGGCCCACCCGAAGGTGAGCCAGTGTGACTCTAGTA |
| | | GAGAGCGTTCACCGACAAACAACAGATAAAACGAAAGGCCCA |
| 86 | IC343-r | GGCCGGTCTCAATTGATACATTTAAAATAATTATGTATTCATGAAACATGATTGT |
| 87 | IC011-r | TTATATACTTGGTTTATTTACTTGATTATTTCTGTAA |
| 88 | IC012-f | TATTGTATAGCTTGGTATCATCT |
| 89 | IC001-f | GAAGACTTAGAAAATTATATGAAAGAAGAAGGT |
| 90 | IC002-r | CAAAAGCACTTGAAAATGCATG |

Having established the functionality of the LAC system, further experiments were undertaken to determine the kinetics of induction, specifically, the time that is required for the system to respond to a change in lactose levels as well as the time-course of induction. Accordingly, C. sporogenes cells harbouring the plasmid pMTL-HZ1, were cultivated in TYG medium in the presence and absence of 10 mM lactose and their optical density (OD600) monitored over time (FIG. 1.4b). Lactose addition was made at 4 hours, when cells had reached the early exponential growth phase (OD600≈0.5); samples were collected at 0, 2, 4, 6, 8, 10, 12 and 24 hours and the level of activity in cell lysates determined (FIG. 1.4a).

Expression of catP above the level of the uninduced culture was detectable immediately upon addition of lactose (at 4 hours), supporting the notion that induction occurs within a few minutes of lactose being added. In agreement with the results obtained by Hartman and colleagues, these data indicate that lactose, and not a derivate, is the metabolite that activates gene expression in C. sporogenes. Activation of gene expression would have not been possible immediately after induction in the case of a lactose derivative, since, generally, some time is required to express the enzymes required for the metabolite conversion and for the conversion itself.

Maximum CAT activity was observed at 8 hours. The levels of reporter began to decrease after 10 hours and had been reduced by approximately 15% after 24 hours. Since no differences in growth rates were observable between the cells grown in the presence and absence of lactose, the observed reduction in expression was not a consequence of a decrease in growth rate caused by the addition of inducer. It is known that the availability of the cell's transcriptional and translational machineries is growth-rate dependent, a factor that can affect gene expression. In particular, this dependency is more pronounced in the case of positive regulation mediated by constitutively expressed activators, since the expression of the activator itself—required to activate the expression of the target gene—is already growth-rate dependant. Basal CAT expression in the absence of the inducer slightly increased with time, which is indicative of the leaky transcription from PbgaR and/or PbgaL.

The LAC system was shown to be functional in C. sporogenes. However, for applications where very tight control of gene expression is required (i.e., to control sporulation), the system remains far from ideal. Of the inducible systems available for Clostridium, only the TET system exhibits very low basal expression while maintaining a high induction efficiency. Nevertheless, the optimal working conditions of the TET system require high doses of the inducer anhydrotetracycline, a tetracycline-analogue. The levels required, however, have significant inhibitory effects on cell growth. Thus, it seemed necessary to obtain (or develop) a more tightly regulated inducible system for Clostridium that does not rely on any antibiotic and is suitable for any potential application.

The strategy adopted in this invention was to synthetically optimize an inducible system, and studies were undertaken to further characterise the LAC system.

### Further Characterization of the LAC System

During transcription initiation, RNAP and one or more transcriptional initiation factors bind to the promoter sequence via sequence-specific interactions to form an RNAP-promoter open complex, initiating transcription at the TSS. Although the distance between the TSS and the promoter core elements can vary, there is a general preference for TSS selection at a position 7 bp downstream of the promoter −10 element. Although there are several tools for predicting promoters available, the vast majority rely on sequence databases from model organisms, such as E. coli, and are not yet very efficient for non-model chassis like Clostridium. Thus, accurate TSS location makes determination of promoters more accurate.

In order to determine the TSS of the promoters involved in the functioning of the LAC system, the approach 5′ RACE, was used. This technique allows the synthesis of cDNA from a specific mRNA target. A polyAtail, termed adaptor, is then added to the 5′ end of the cDNA, which allows further amplification and determination of the sequence of the 5′ UTR. Analysis of the bgaR promoter required a new reporter vector containing PbgaR upstream of the catP reporter, termed pMTL-IC1201. As with previous reporter constructs, it is based on the pMTL82251 chassis. The general LAC reporter vector, pMTL-HZ1, was used to determine the TSS of PbgaL. Total RNA was extracted from stationary cultures of C. sporogenes harbouring the designated constructs and 5′ RACE was performed using catP specific primers for the cDNA reverse transcription and extension steps (FIG. 1.6). The extension step also required the utilisation of a primer that binds to the adaptor, named anchor primer. As a result, the amplification of the 5′ UTR sequence downstream of PbgaL and PbgaR, was successfully obtained (FIG. 1.7).

Sequencing of the PCR products resulted in 5′ UTR sequences with the TSS located 66 and 109 nucleotides upstream of the translation start site (ATG) of PbgaR and PbgaL, respectively. It has to be noted that one limitation of the 5′ RACE procedure is the inaccurate determination of the TSS of mRNAs ending with one or several thiamine residues, since during 5′ RACE a poly-A tail adaptor is added to the cDNA 3′ end (complementary to the 5′ UTR in the mRNA). Therefore, discrimination between a thiamine residue belonging to either the original 5′ UTR sequence, or to the anchor primer that binds to the poly-A adaptor, is impossible. Following the rule of the 7 bp upstream of the TSS, and based on the consensus 17-bp spacer between the −10 and −35 regulatory elements, the putative −10 and −35 boxes were manually annotated. It is worth noting that, despite the determination of the putative −10 and −35 elements being possibly accurate, they can only be approximations, since the analysis of the importance of every nucleotide residue nearby would be required for an exact determination.

Regulation of gene expression in response to inducing ligands (i.e., lactose) is mainly mediated by TFs. These interact with conserved cis-acting elements, or operator motifs, that are located in close proximity to or within the promoter sequence of genes involved with the metabolism of the inducer. Since bgaR encodes the TF that activates gene expression in the LAC system, the operator sequence/s linked to this regulation should be situated in close proximity to the annotated regulatory elements of PbgaL. One simple approach to identify regulatory elements within the promoters of regulated genes is to compare sequences from divergent species. By multiple alignment, conserved elements can be distinguished from sequences that have evolved more rapidly. Accordingly, an approach to predict TF-specific motifs, previously reported by Francke and colleagues, but applied to a different family of TFs, was chosen. The main premise behind this approach is that equivalent regulatory sites will be shared only if orthologous sequences share genomic context. In this sense, synteny—conserved gene order—was considered to strongly indicate functional equivalency. Thus, only TFs divergently oriented in relation to the regulated gene were considered in this study. Since BgaR (accession number WP_003480500), is classified as an AraC/XylS-famly of TFs, a total of 71 non-redundant AraC/XylS-family TFs from taxonomically related Firmicutegenomes were aligned and grouped through a Neighbor Joining algorithm using the program MEGA version 6 (FIG. 1.8).

The 300 bp upstream of the most phylogenetically related sequences (highlighted in black) were used to identify consensus motifs using the program MEME. As a result, two consensus motifs of 20 bp in length, termed O1 and O2, were identified as hypothetical regulatory elements of the LAC system (FIG. 1.9). FIG. 1.10 illustrates the LAC system from C. perfringens strain 13 and the hypothetical operator sequences O1 and O2.

Following the identification of O1 and O2 as hypothetical operator sequences, 3 new constructs were designed in which either O1 or O2 and both O1 and O2 had been deleted, generating vectors pMTIL-ICMO1, pMTL-ICMO2 and pMTL-ICMO102, respectively. Additionally, a construct lacking bgaR, termed pMTL-ICMB, was also created, which served to determine to what extent activation of gene expression depended on the presence of BgaR.

The CAT data shown in FIG. 1.11 confirmed that O2, or part of it, is a key regulatory sequence for the functionality of the LAC system. The deletion of O2, alone or in combination with O1, led to a severe reduction of gene 81 expression and no differences were observable when lactose was present or absent. The same expression profile was found when bgaR was deleted, indicating that the system requires bgaR for the activation of gene expression. In contrast, deletion of the hypothetical operator sequence O1 did not result in a significant difference to gene expression when compared to the native system in the presence of the inducer (unpaired two-tailed Student's 1-test). Interestingly, this was not the case in the absence of lactose, evidenced by a significant difference in background expression between the construct lacking O1 and the native system. According to previous results, O1 is located 29 bp upstream of the TSS (distance relative to the 10th base of the 20 bp of O1) and overlaps the determined putative −10 regulatory element. It is possible that the deletion of O1 affects the promoter region of bgaR, which by pure coincidence, results in an increased expression of bgaR, and as a consequence, of the background level of expression. In all other cases, background expression was slightly higher than that of the promoter-less reporter vector but lower if compared to the native system. Since this also occurs in the absence of bgaR, where background expression was reduced by 50%, it suggests that this basal expression is the result of the leaky transcription from PbgaL. In the same context, promoter leakage of the native system is likely the result of the combinational noise derived from both, PbgaR and PbgaL.

Having established that O2 might be involved in PbgaL activation, further investigations of the importance of the DNA sequence around this motif were pursued via mutational analysis. At this point, it was considered that O2 is not part of the main regulatory elements of PbgaL (−10 and −35 elements), since O2 is located at a distance of 59 bp from the TSS downstream of PbgaL (between positions −72 and −52). Several pMTL-HZL1-derived mutant plasmids with truncated or mutated sequences located upstream of the TSS were therefore generated employing a previously reported mutagenesis strategy; PbgaL activity was quantified by determining levels of CAT activity in lysates of plasmid carrying C. sporogenes strains. A total of eleven truncated variants were constructed, termed pMTL-ICT1 to −T11, by sequentially deleting five nucleotides between positions −89 and −34 (where the −35 element resides) (FIG. 1.12a). Additionally, a total of eleven mutated variants were constructed, named pMTL-ICM1 to −M11, whereby blocks of five nucleotides were substituted by their complementary base pairs, between positions −89 and −34 (FIG. 1.12b).

Truncations downstream of position −69 (pMTL-ICT5), within the 5′ sequence of PbgaL caused a strong decrease in promoter activity from position −69 pMTI-ICT5). In particular, deletions downstream of position −64 (pMTL-ICT6) resulted in the same level of gene expression previously observed when deleting the consensus motif O2 or the transcriptional factor bgaR, which corresponds to the background expression of PbgaL. Additionally, substitutions of 5 bp length within the same region provided extra information. Mutations between positions −89 and −74 did not have a significant effect on PbgaL activity, relative to the native system. Substitutions between positions −74 and −69 slightly decreased gene expression (pMTL-ICM4), whereas those occurring between positions −69 and −44 had a more severe effect on PbgaL expression. In some cases, such as in the substitutions between positions −69 to −64 and −59 to −54, corresponding to vectors pMTL-ICM5 and pMTL-ICM7, respectively, gene expression was almost abolished. Interestingly, substitutions between the positions −44 and −39 did not have any effect on PbgaL expression (pMTL-ICM10), whereas the five nucleotide substitution immediately after this (between positions −39 and −34, pMTL-1CM 11) resulted in a level of gene expression comparable to the background. This result supports the validity of the previously determined position of the putative −35 element, which is partially deleted in pMTL-ICM11. This finding also agrees with O1 being a conserved consensus motif—the −35 element—of the sequences previously analysed.

The data presented here provides a general outline of the mechanisms behind the LAC system from C. perfringens strain 13, and allowed the generation of a synthetic system with both reduced background expression and large dynamic range.

### Example 2—Theophylline-Responsive Translational Riboswitches in Clostridium

In this Example, it is shown that previously existing, as well as newly designed theophylline-dependent riboswitches, function in C. sporogenes with a response that is dependent on inducer concentration.

### Theophylline Responsive Riboswitches for Clostridium

As previous work had shown that many diverse theophylline-dependent riboswitches were functional in both Gram-positive and Gram-negative bacteria, it was decided to evaluate the performance of this inducible translational regulatory system in C. sporogenes NCIMB 10696. Accordingly, three riboswitch sequences, riboswitch-D, -E and -E*, which were expected to exhibit higher translation efficiencies after induction, out of the six theophylline responsive riboswitches developed by Gallivan and coworkers were selected. In previous publications, riboswitch-E* was shown to be the best theophylline-dependant riboregulator in Gram-positive bacteria, thanks to its high dynamic range and its very low basal expression. In the present study, 4 new riboswitches (named -F to -I) were constructed by rationally modifying the space between the SD sequence and the translational start site of the original riboswitch-E* (Table 4).

TABLE 4
Nucleotide sequences of the riboswitches
used in this study. The aptamer sequence
is underlined. The translational start is
bold and italicised. Modifications of the
parent riboswitch-E* are indicated in red,
Rational modifications were based on known
strong RBS sequences in the genus Clostridium.

| SEQ | | |
|---|---|---|
| ID NO. | Riboswitch | Sequence (5′ [symbol] 3′) |
| 4 | E | GGUGAUACCAGCAUCGUCUUGAUGCCCUUGGCA |
| | | GCACCCUGCUAAGGAGGUAACAACAAG [symbol] |
| 7 | G | GGUGAUACCAGCAUCGUCUUGAUGCCCUUGGCA |
| | | GCACCCUGCUAAGGAGGUAACUUA [symbol] |
| 8 | H | GGUGAUACCAGCAUCGUCUUGAUGCCCUUGGCA |
| | | GCACCCUGCUAAGGAGGUGUGUUA [symbol] |

A reporter plasmid, pMTL-IC101, derived from the vector pMTL82251, was used as a chassis for all of the subsequently created plasmids (FIG. 2.2). This backbone contains the native promoter of the C. sporogenes ferredoxin gene (Pfdx, associated with the protein coding gene Clspo_c0087) upstream of the reporter catP and serves as a reference to compare CAT expression measurements. pMTL-IC101 also contains the same elements as the reporter vectors used in Example 1, pMTL-HZ1 and pMTL-IC001, including the pBP1 Gram-positive origin of replication, the Gram-negative ColE1 origin of replication, the erythromycin resistance gene (ermB) and the TCD0164 and Tfdx terminators, respectively derived from the C. difficile CD0164 and C. sporogenes ferredoxin genes, here flanking the promoter-gene sequence. Due to this compatibility, the promoter-less backbone previously generated, pMTL-IC001, was also used to detect background expression in the experiments carried out in this Example.

As a default, the riboswitches, preceded by a linker sequence, were genetically fused to the core region (−35 and −10) of the strong Pfdx. just downstream of the TSS and excluding the native 5′ UTR sequence, as previously described 187. The sequence comprising the promoter-riboswitch-reporter was then cloned into the aforementioned reporter chassis.

In order to place the riboswitch sequences downstream of the TSS of Pfdx, the TSS of Pfdx had to be determined. To this end, the 5′ RACE procedure described in Example 1 was used. Total RNA was extracted from stationary cultures of C. sporogenes harbouring the reporter plasmid pMTL-IC101 and 5′ RACE was performed using the same catP specific primers described in Example 1. As a result, the amplification of the 5′ UTR sequence downstream of Pfdx was successfully obtained (FIG. 2.3).

Plasmid constructs, designated pMTL-IC111-D to -J, were conjugated into C. sporogenes and cultures of the resultant transconjugant cells exposed to 2 mM theophylline inducer when they had reached the early exponential phase of growth (OD600≈0.5). CAT activity was determined in cell lysates derived from stationary phase cultures cultivated in the presence or absence of the inducer (FIG. 2.4). The synthetic theophylline responsive riboswitch is composed of an aptamer and a synthetic SD (FIG. 2.5). Theoretically, transcription of the riboswitch under the control of Pfdx occurs in a constitutive manner during cell growth. However, the synthetic SD sequence located downstream of the aptamer is sequestered via pairing with the stem of the riboswitch, resulting in translational block. The binding of theophylline releases the SD by altering the downstream base pairing. As a consequence, gene translation occurs when the ribosome binds to the SD86. As shown in FIG. 4.4a, strains harbouring riboswitches -E, -E*, -F, -G, -H and -I exhibited statistically significant induction in C. sporogenes after addition of 2 mM theophylline to the culture. In particular, riboswitch G outperformed previous theophylline-dependant riboswitches, demonstrating higher levels of CAT activity, low basal expression and the strongest activation ratio (FIG. 4.4b). In all cases, the incorporation of any riboswitch in the 5′ UTR led to a strong reduction in CAT activity; this agrees with previously published studies, indicating that secondary structures near the RBS play a major role in the translation of the downstream mRNA86,186,187. In agreement with the assays performed in Example 1, CAT activity of cells harbouring the control pMTL-IC001 was similar to that from cultures lacking the reporter backbone (WT), indicating no detectable transcriptional read-through and demonstrating the suitability of the selected backbone as an insulated chassis for the construction of switchable systems.

### Adjustability of the Theophylline Responsive Riboswitches

Due to the importance of having inducible systems where gene expression can be controlled within a large regulatory window, it was decided to compare the expression pattern of riboswitches -E, -G and -H when placed downstream of the core region of a synthetically weakened Pfdx, named Pfdx4, creating vectors pMTL-IC201-E, -G and -H. The promoter Pfdx4 was generated by replacing the core elements −10 and −35 of Pfdx with the same regulatory elements of the constitutive promoter from the C. acetobutylicum ATCC 824 ptb gene (Pptb, associated with the protein coding gene Ca_3076) (FIG. 2.6a). To further examine the regulatory response of the theophylline responsive riboregulators in clostridia, riboswitches -E, -G and -H were additionally fused to the promoters Pfdx and Pfdx4, retaining the bases downstream of the TSS but excluding their native SD sequences. These sequences, named Pfdx* and Pfdx4*, maintain the full upstream region, including the core region (−35 and −10), the TSS and the space between the TSS and the native SD. Constructs, named pMTL-IC121-E, -G and -H1 (for Pfdx*) and pMTL-221-E, -G and -H (for Pfdx4*), were designed to express the reporter gene catP, conjugated into C. sporogenes and the growing cultures of the transconjugants obtained exposed to 2 mM theophylline inducer or grown without induction. Since the linker sequence located between Pfdx and the riboswitch slightly differs from that placed between Pfdx* and the riboswitch, FIG. 2.7 shows the exact nucleotide residues of both linker sequences.

As shown in FIG. 2.6b, the combination of different riboswitches with various promoters and two different 5′ UTRs allowed the expansion of the regulatory range, providing a library of theophylline-inducible switches suitable for applications where protein yield is crucial as well as for the expression of detrimental or toxic proteins. The combination of any riboswitch with the weak promoter Pfdx4 led to the lowest expression levels. This was also the case when the native 5′ UTR sequence was retained, establishing a direct correlation between detected CAT activity and promoter strength (Pfdx>Pfdx4). However, basal expression appears to be compromised when high expression levels are achieved, resulting in increased leakiness in those constructs that exhibited higher expression levels (i.e., with Pfdr*-G). Expression of catP was always higher when the native sequence downstream of the TSS was maintained. Since riboswitch-G showed the highest induction level of the analysed riboregulators, it was subjected to further characterization.

Functional Characterization of the Theophylline Responsive Riboswitch in C. sporogenes NCIMB 10696

To assess the dose dependency of riboswitch regulation, strains harbouring plasmids that carried the riboswitch-G downstream of Pfdx or Pfdx* were cultivated with increasing concentrations of theophylline, ranging from 0.1 to 10 mM (FIG. 2.10a), and the optical density (OD600) of the cultures monitored over time (FIG. 2.10b). Increasing doses of theophylline would be expected to result in higher levels of gene expression. Concentrations higher than 5 mM, however, resulted in a reduction of growth, in agreement with previously reported results in other Gram-positive and Gram-negative bacteria. The translational switches were also analysed for CAT expression over time in the presence and absence of 2 mM theophylline. As shown in FIG. 4.10c, CAT expression above the level of the uninduced culture was already detectable 30 minutes after induction (4.5 hours of growth). The maximum level of reporter gene expression was reached at 8 and 10 hours when using Pfdx and Pfdx*, respectively. Although both have a similar profile of induction, after 24 hours, CAT expression was reduced by 75% in Pfdx whereas only by 40% in Pfdx* Given that in both cases the translated product is the same (CAT), these results suggest that the reversal of induction is strongly dependent on the abundance or stability of the mRNA.

As with lactose in Example 1, it was decided to establish whether theophylline is metabolized by C. sporogenes. C. sporogenes cells were, therefore, cultivated in TYG medium supplemented with 2 mM theophylline and the concentration of theophylline was monitored by HPLC/MS analysis of cell free supernatant samples over time. Results showed that the concentration of theophylline remained constant over the course of the experiment, with a slight increase in the culture supernatant 20 hours after induction, possibly due to the release of the inducer from the cells after lysis (FIG. 2.10d).

Altogether, these data demonstrate that the theophylline responsive riboswitches function in C. sporogenes with a response that is dependent on inducer concentration. In particular, the rationally designed riboswitch-G outperformed previously published riboswitches, exhibiting very low basal expression and the largest dynamic range. The regulatory window of the theophylline responsive riboswitches could be adjusted by modifying the 5′ UTR sequence located upstream of the riboswitch or by using promoters with different strengths. However, the incorporation of any riboswitch in the 5′ UTR led to a strong reduction in reporter activity in all cases, with higher levels of gene expression linked to increased leakiness in the absence of the inducer. This, in fact, compromised the dynamic range of the riboregulators. In all cases, the riboswitch was able to respond to the inducer theophylline, ensuring its performance independent of the genetic context. Additionally, because C. sporogenes does not degrade theophylline, this is confirmed, thus far, as an appropriate inducer for further applications.

### Example 3—A Dual Mechanism to Tightly Control Gene Expression (Ribolac)

Having established the limitations of the existing transcriptional inducible systems to tightly control gene expression in Clostridium, it was decided to engineer a system with improved characteristics so that high levels of gene expression do not compromise the dynamic range. By combining the theophylline responsive riboswitch with the transcriptionally regulated LAC system from C. perfringens strain 13, a dual translational-transcriptional ON device that controls the expression of both, bgaR and the gene of interest, was generated. In this system, termed RiboLac, optimal expression of the target gene requires induction with both theophylline and lactose.

RiboLac, a Dual Mechanism with Improved Dynamic Range

In Example 1, it was demonstrated that the LAC system requires BgaR to activate gene expression. It was also shown that background expression was reduced by 50% if the system lacks BgaR, providing a clue as to how leaky expression could potentially be reduced in this inducible system. Additionally, some of the riboswitches characterized above, in particular riboswitches -E, -G and -H, demonstrated tight control of gene expression in the OFF state, exhibiting diverse expression profiles upon activation. By combining these two types of regulation, it was hypothesized that a system with translational control over bgaR and transcriptional control over the target gene (catP) could be generated, in which expression was subject to the addition of two inducers, lactose and theophylline. Since the amount of BgaR required to activate gene expression in native inducible system was unknown, it was decided to generate several systems that would lead to various levels of BgaR in both the ON and OFF states. It was expected that one of the systems would allow an adequate expression of BgaR in the presence and absence of the inducer, successfully facilitating controlled activation of PbgaL. Accordingly, three riboswitch sequences, riboswitch-E, -G and -H, were genetically fused to the core region (−35 and −10) of the bgaR promoter, PbgaR, just downstream of the TSS (determined in Example 1) and excluding the native 5′UTR, generating vectors pMTL-ICE1, pMTL-ICG1 and pMTL-ICH1. The rest of the system remained as it was in the parent LAC reporter vector pMTL-HZ1. Additionally, the same riboswitches were placed downstream of PbgaR*, which retains the bases downstream of the TSS but excludes the native SD sequence, as described in Example 2, generating the vectors pMTL-ICE1*, pMTL-ICG1* and pMTLICH1*. Constructs, which contained the reporter gene catP, were conjugated into C. sporogenes and growing cultures of the transconjugants obtained were exposed to either one of the inducers—theophylline or lactose—to both inducers, or grown without induction.

The CAT data shown in FIG. 3.1 demonstrates that the dual theophylline-lactose system, termed RiboLac, responds to both inducers and generates a response that is dependent on BgaR expression. In RiboLacs containing the riboswitch downstream of PbgaR, statistically significant expression of catP above the level of the uninduced culture was achieved only in the presence of both inducers, indicating that these dual systems act as Boolean AND logic gates (FIG. 3.1a). Specifically, the RiboLac harbouring riboswitch-E (pMTL-ICE1) showed the highest dynamic range (approximately 13-fold), out of the three systems containing PbgaR. In all three cases, expression after induction was severely reduced if compared with the native system, which despite exhibiting considerable background expression, still afforded a higher dynamic range (approximately 15-fold). Conversely, significantly increased expression of catP was observed in RiboLacs with riboswitches located downstream of PbgaR* when cultures were supplemented with only one of the inducers (FIG. 3.1b, asterisks not shown). However, expression after induction with one inducer was lower than that of the LAC system in the presence of lactose, indicating that the system is capable of dual regulation of PbgaR and PbgaL at the translational and transcriptional levels, respectively. Interestingly, the system containing riboswitch-G downstream of PbgaR* exhibited very low basal expression in relation to the native system, and maximum expression was not compromised after induction with both inducers. As a consequence, the dynamic range increased from approximately 15-fold in the native system to approximately 41-fold in this specific RiboLac. As expected, theophylline did not have any effect on the expression of the LAC system. Data from this study indicate that expression of BgaR under the control of a riboswitch located downstream of PbgaR is lower than the expression of the same protein when the riboswitch is placed downstream of PbgaR*, both in the presence and absence of the inducer. Repression of the transcriptional activator in the OFF state is very tight in systems containing PbgaR; induction, however, did not result in the high level of expression observed in the native system. By contrast, systems containing PbgaR* achieve the same levels of expression as the native system in the presence of inducers, subject to higher basal levels of gene expression. These results also agree with the data obtained when characterizing the theophylline responsive riboswitches in Example 2, where the same pattern of expression between Pfdx and Pfdx* (or Pfdx4 and Pfdx4*) was observed. Since the dual system containing Pfdx*-G showed the highest induction level out of the analysed systems, it was decided to characterize it further. Henceforth, the term RiboLac will be used to refer to this particular system in this and subsequent Examples.

### Functional Characterization of the RiboLac

Having established the functionality of the RiboLac, further experiments were undertaken to determine the dynamics of induction; specifically, the activation of PbgaL when bgaR expression is induced with various concentrations of theophylline. Accordingly, strains harbouring the plasmids pMTL-HZ1 and pMTL-ICG1* were cultivated with increasing concentrations of theophylline ranging from 0 to 2 mM, in the presence and absence of 10 mM lactose (FIG. 3.2). Increasing doses of theophylline would be expected to result in higher levels of BgaR, and therefore, increased levels of CAT upon PbgaL activation. This was the case when the system was induced with theophylline alone. Although little change in induction was observed between 0.5 and 1 mM theophylline, addition of 2 mM resulted in a higher PbgaL activation. In no case did induction with one inducer achieve maximum expression levels. Interestingly, the response of the RiboLac seemed to be peak when induced with 0.5 mM theophylline in the presence of lactose, with levels of gene expression similar to those obtained at maximum induction (2 mM theophylline and 10 mM lactose). The expression of RiboLac-dependent catP in the presence of lactose alone, which is lower than that of the LAC system under the same conditions, seems to be the result of PbgaL activation when the levels of BgaR are below the saturation capacity of the system.

Additionally, the RiboLac was also analysed for CAT expression over time in the absence of inducers and at the highest concentrations of inducers tested. In the previous theophylline-dependency assay, maximum induction was obtained with 0.5 mM theophylline and 10 mM lactose. However, in Example 2, maximum induction of the theophylline responsive riboswitches, without affecting cell growth, was obtained with 2 mM theophylline. As a matter of consistency, 2 mM theophylline was used to obtain maximum levels of expression in the RiboLac as well. Accordingly, CAT expression of cultures grown in the presence and absence of 2 mM theophylline and 10 mM lactose, as well as their optical density (OD600), were monitored over time (FIG. 3.3a and FIG. 3.3b).

In both systems, expression of catP above the level of the uninduced culture was detectable immediately upon addition of the inducers (at 4 hours), indicating that expression of BgaR under the control of the riboswitch occurs within a few minutes of theophylline being added. No significant differences in CAT expression were observed between the LAC system and the RiboLac in the presence of inducers. However, whilst CAT expression in the absence of inducer slightly increased with time in cultures harbouring the LAC system, from six hours onwards, it decreased in cultures harbouring the RiboLac. Maximum expression under the control of the theophylline responsive riboswitch studied in Example 2 was reached between 4 and 6 hours post induction (8-10 hours of growth), with expression after 20 hours reduced by 75% and 40% in Pfdx and Pfdx*, respectively. Accordingly, reversal of background expression in the RiboLac over time might be associated with the abundance or stability of the mRNA encoding BgaR, which is strongly reduced between 4 and 6 hours after induction. No differences in growth rates were observed between the cells grown in the presence and absence of the inducers.

### The RiboLac in the Chromosome

The engineering of plasmid-free stable strains requires that the biological parts conferring the desired functionality are integrated into the host genome. To determine whether the degree of repression and dynamic range would provide suitable levels of gene expression under these conditions, the RiboLac was integrated into the clostridial host chromosome. When this study initiated, chromosomal integration into clostridial genomes was achieved via recombination-based allelic exchange. Specifically, integration of a particular cargo into the same strain of C. sporogenes used in this study had been previously been accomplished using ACE, relying on the interconversion of the pyrE locus between a functional and mutant allele. In ACE, the coupling of a plasmid specified allele with a chromosomal allele during allelic exchange results in the formation of a new allele that has a selectable phenotype. The pyrE gene encodes the enzyme orotate phosphoribosyltransferase which is involved in de novo synthesis of the pyrimidine base, uracil. Cells carrying a mutant, chromosomally located pyre allele, lacking the 3′ end of the gene, can no longer make uracil, and are unable to grow in defined media without the addition of exogenous uracil. ACE plasmids carrying a pyrE allele comprising all but the first 8 codons of the gene can be used to restore the mutant, chromosomal allele to WT through selection for uracil prototrophy. Moreover, cargo DNA inserted into the ACE plasmid immediately downstream of the pyrE allele becomes integrated into the genome concomitant with restoration of prototrophy.

To integrate RiboLac in the pyrE locus, the vector pMTL-JH29, which contains two unequally sized regions of homology to the C. sporogenes chromosome, was modified to include a double terminator immediately downstream of the pyrE gene, intended to insulate the integrated expression unit. The double terminator, composed of the E. coli rrnB T1 terminator and the phage T7 early transcription terminator, was synthesised based on the existing sequence in the registry of standard biological parts (BBa_BOO15). The terminator rrnM T1 from E. coli had been demonstrated to be functional in C. acetobutylicum, which, combined with the phage T7 early transcription terminator, is the most frequently used double terminator from the registry of standard biological parts. The integration vector containing the double terminator was renamed pMTL-JH29T. In this vector, one of the homologous regions, corresponding to the longer 1200 bp region immediately downstream of pyrE, directs the first recombination event resulting in single crossover integration of the plasmid. Subsequent plasmid excision via the second, smaller region of homology, corresponding to a pyrE allele lacking its 5′ end, results in the integration of the double terminator concomitant with restoration of uracil prototrophy. The vector includes the restriction sites NotI and XhoI between the double terminator and the longer homologous region so that a specific cargo can be easily inserted in the vector for its consequent integration. Accordingly, two vectors, pMTL-JH29T-L and pMTL-JH29T-R, were created by inserting the LAC system-catP and the RiboLac-catP coding sequences, respectively. These sequences were directly taken from the reporter vectors pMTL-HZ1 and pMTLICG1*, respectively, through restriction digestion using the restriction sites NotI and XhoI. Inserts were incorporated into the integration vector via the same restriction sites. As such, the LAC and RiboLac coding sequences also include the native terminator downstream of bgaR. The designated plasmid constructs were transferred into C. sporogenes (in triplicates) from their respective E. coli donor strains. Integrants were isolated by plating cultures of the resultant transconjugants onto defined medium lacking uracil. Integration of both the LAC system and the RiboLac reporter systems into the pyrE locus were confirmed via PCR, using external primers targeting the C. sporogenes chromosome (FIG. 3.4), and subsequent DNA sequencing of the amplified DNA fragments. A second PCR product was obtained in all cases, including the WT strain, indicative of non-specific amplification using the selected pair of primers.

In order to assess expression, cultures of the integrants obtained, named C. sporogenes::LAC and C. sporogenes::RiboLac, were cultivated in TYG medium and induced with only one of the inducers, theophylline (2 mM) or lactose (10 mM), or with both inducers (2 mM theophylline and 10 mM lactose) as well as in the absence of inducers. A control culture was included to which no inducers were added. Strains harbouring the reporter plasmids pMTL-HZ1 and pMTL-ICG1* were used as positive controls as well as to determine the differences in expression encountered between the plasmid-based systems and the same systems integrated into the clostridial chromosome.

Expression of catP driven by the integrated RiboLac was only detectable in the presence of both inducers, indicating that the dual mechanism is functional also in the chromosome of C. sporogenes (FIG. 3.5). Expression in the absence of inducers as well as in the presence of only one inducer were comparable to that of the WT strain. The level of CAT observed when C. sporogenes::RiboLac was induced with both, theophylline and lactose, was similar to that of C. sporogenes::LAC in the presence of lactose. As expected, maximum expression was markedly reduced if compared with cells harbouring the plasmids pMTL-HZ1 and pMTLICG1*, being reduced by approximately 10-fold when the systems were integrated into the single copy clostridial chromosome. No differences in background catP expression were shown between C. sporogenes::LAC and C. sporogenes::RiboLac. Considering that differences were observable when the systems were tested in a plasmid (FIG. 3.2 and FIG. 3.3), it might be possible that the CAT assay used in this study is not sensitive enough to detect the low levels of expression obtained from the single copy chromosomal integration. It is expected, however, that the integrated RiboLac is likely to be providing tighter control of gene expression than the integrated native LAC system.

### Example 4—Development of a Conditionally Sporulating C. sporogenes Strain

In this Example, the inducible systems described above are utilized to generate conditionally sporulating strains by controlling the expression of the master regulator Spo0A. After demonstrating that plasmid based conditionally sporulating strains could be created, stable strains with inducible control of spore formation integrated into the bacterial chromosome were generated. Specifically, by addition of the inducers lactose and theophylline, the strain C. sporogenes::RiboLac-spo0A can be artificially induced to sporulate, with a sporulation capacity similar to that of the WT strain. This strain is completely asporogeneous in the absence of inducers.

### Generation of an Asporogeneous Host to Test Conditional Sporulation Systems

spo0A represents one suitable target for the creation of a conditionally sporulating strain. As such, a strategy wherein the inducible systems from Examples 1 and 2 are used to regulate the expression of spo0A was considered. Since the systems exhibited various dynamic ranges, it was expected that one of them would result in a strain incapable of sporulating in the absence of induction but able to restore spore formation—ideally to WT levels—in the presence of inducer/s.

In order to generate a conditionally sporulating strain via tightly regulating the expression of the spo0A gene, a strain deficient in this gene had to be generated. Deletion of the spo0A gene (Clspo_c18650) was achieved via creating an in-frame deletion by pyrE-based allelic exchange. pyrE-based allelic exchange also requires a pyrE-deficient background. With the PyrE enzyme truncated, the organism is uracil auxotrophic and resistant to the compound 5-FOA. The selection of mutants is achieved by using a heterologous pyrE gene as a negative counter-selection marker in the presence of 5-FOA. Additionally, since the in-frame deletion is achieved in a pyrE-deficient background, complemented strains can be easily obtained using ACE whilst the pyrE allele is corrected.

The isolation of the single cross-over plasmid integrants is facilitated by “pseudo-suicide” vectors. These are plasmids that carry a selection marker (i.e., catP, encoding resistance to thiamphenicol) which are sufficiently defective in its replication so that cells carrying the non-integrated plasmid exhibit a growth disadvantage relative to cells in which the vector, containing the gene that confers resistance to the antibiotic (i.e., catP), has been integrated. As such, clones with the plasmid integrated grow faster on agar medium supplemented with thiamphenicol than cells carrying catP on a non-integrated, replication defective plasmid because if integrated growth is not limited by the rate at which the plasmid is segregated among the progeny. A vector derived from the construct pMTL-CH14, which had been previously generated by a member of this group, was used to delete the spo0A gene from the C. sporogenes pyrE- strain. The plasmid pMTL-CH14 incorporates a heterologous, functional pyrE gene from C. acetobutylicum and the C. difficile pCD6 replicon. Since the origin of replication from the Bacillus plasmid pIM13 was shown to be more appropriate for the isolation of faster-growing single-colonies in the same strain of C. sporogenes, the pCD6 replicon was replaced by the pIM13 replicon in pMTL-CH14, generating the deletion vector pMTL-ICD. The spo0A knockout cluster, comprising two equal 1000 bp homologous regions upstream and downstream of spo0A, respectively, was generated by SOEing PCR and inserted into the deletion vector. The deletion vector, termed pMTL-ICD-spo0A, was transferred into C. sporogenes (in triplicates) from its respective E. coli donor by conjugative plasmid transfer; transconjugant cells were screened for plasmid integration by colony PCR (FIG. 4.1).

Successful single-cross mutants derived from three independent conjugations—all obtained by homologous recombination through the left homologous regions (LHR)—were streaked onto TYG medium supplemented with 5-FOA; deletion of the spo0A gene was confirmed via colony PCR from S-FOA-resistant colonies, using primers aligning outside of the crossover region (FIG. 4.2), and subsequent Sanger sequencing. The mutant strain, named C. sporogenes Δspo0A pyrE-, was subject to complementation and pyre restoration for its appropriate characterization.

spo0A Complementation and pyrE Repair

In order to confirm that the phenotype observed in the mutant strain is directly attributable to the deletion of spo0A, a spo0A complemented strain was generated via ACE, incorporating spo0A under the control of its own promoter downstream of the repaired pyrE locus. The integration plasmid pMTL-JH29-spo0A, carrying spo0A flanked by its native promoter and terminator, was conjugated into C. sporogenes Δspo0A pyrE- and integrants were isolated by plating cultures of the resultant transconjugants onto defined medium lacking. The complemented strain was named C. sporogenes Δspo0ACOMP. Additionally, in order to fairly compare the spo0A mutant with the complemented strain, which contains a functional pyrE gene after being complemented with spo0A in the pyrE locus, a strain lacking spo0A but with a functional pyrE gene was also created. Accordingly, the “empty” pMTL-JH29 vector (with no integration cargo) was used in a similar fashion to restore the full-length of the pyrE gene, generating C. sporogenes Δspo0A.

Characterization of C. sporogenes Δspo0A pyrE-

C. sporogenes Δspo0A pyrE-, the strain which would serve as a host to create a conditionally sporulating strain, was characterized for both its sporulation capacity and growth profile. Accordingly, the spo0A complemented strain, C. sporogenes Δspo0ACOMP was used to evaluate if the deletion of spo0A is the cause of the expected asporogeneous phenotype. The spo0A mutant with regained uracil autotrophy, C. sporogenes Δspo0A, was included in the assay to account for any effect pyrE could have on spore formation and growth. C. sporogenes WT and C. sporogenes pyrE- were used as positive controls. Detection of heat resistant spores follows a simple procedure of heat-shocking cell suspensions at 80° C. for 20 minutes; CFUs are obtained from plating serial dilutions of such suspensions on appropriate nutrient agar plates. Accordingly, synchronized cultures of the designated strains were cultivated in TYG medium and total and heat-resistant CFU were enumerated at 24 hour intervals for five days (FIG. 4.4a). The optical density (OD600) of the same cultures was monitored during the first 24 hours of cultivation (FIG. 4.4b).

As anticipated, none of the spo0A mutants, pyrE- and pyrE-repaired, were able to form spores over the course of the experiment; in both cases, total CFU was significantly lower than in the strains containing spo0A (approximately 10-fold) (unpaired two-tailed Student's t-test, *p≤40.0332 in both cases), indicative of the role Spo0A plays in cell viability. Moreover, a slight decrease in cell density was observed after 24 hours of growth in strains lacking spo0A. This would be in line with the notion that a mechanism (i.e., phosphorelay in B. subtilis), at the centre of which is Spo0A, integrates nutritional, cell density and other signals to initiate the sporulation process 226. The complemented strain, C. sporogenes Δspo0ACOMP, was able to sporulate; however, its sporulation capacity was reduced by approximately 10-fold at all time points when compared to the spo0A-positive strains.

### Plasmid-Based Conditionally Sporulating Strains

Having generated the strain of C. sporogenes required to test if the inducible expression of spo0A would result in the conditional control of spore formation, it was decided, initially, to evaluate the sporulation capacity of C. sporogenes Δspo0A strains harbouring plasmids where spo0A expression was regulated by the inducible systems developed in the previous Examples; these are: theophylline responsive riboswitches, the LAC system from C. perfringens strain 13 and the RiboLac.

To facilitate comparisons of the results obtained in the sporulation assays and to estimate how the reported strengths and dynamic ranges of the inducible systems relate to spore formation when controlling spo0A expression, CAT assays to monitor inducible promoter activities using all the systems generated in the Examples were carried out (FIG. 4.6a). Considering that the incorporation of any riboswitch in the 5′ UTR downstream of a promoter led to a strong reduction of gene expression (FIG. 2.6, Example 2), and that expression would be further reduced when generating stable strains with spo0A incorporated into the single copy bacterial chromosome, the very strong constitutive promoter of the Clostridium acetobutylicum araE gene (ParaE, associated with the protein coding gene Ca_1339) was included in the repertoire of theophylline responsive riboswitches. Since gene expression levels are higher when the native 5′ UTR is retained upstream of the riboswitch, and due to the easy construction of riboswitches located downstream of a SD-deficient promoter, only ParaE*, and not ParaE, was considered as a candidate to generate a conditionally sporulating strain. The reporter vectors containing ParaE* upstream of riboswitches -E, -G and -H were created similarly to the constructs described in Example 2, generating vectors pMTL-IC321-E, -G and -H and strains harbouring these plasmids were included in the CAT assay.

For assessing sporulation, the backbone pMTL82251 used in the previous Examples was also considered as a backbone for all the spo0A expressing plasmids. All vectors were assembled in an identical fashion to the CAT reporter plasmids, but with spo0A as the target gene instead of catP. The series of spo0A expressing plasmids, termed pMTL-ICSPO followed by the name of the regulatory system (e.g., pMTL-ICSPO-Pfdx4-E for the vector expressing spo0A under the control of PJdx4 located upstream of riboswitch-E), were conjugated into C. sporogenes Δspo0A from their respective E. coli donors. Cultures of the resultant transconjugants were analysed for heat resistant spore production after 120 hours of nutrient starvation in liquid culture, in the presence and absence of the inducer/s. This is normally considered as the time after which the sporulation process in most of the cells of a C. sporogenes culture has been completed 228. C. sporogenes WT and C. sporogenes Δspo0A were used as positive and negative controls, respectively.

Results from FIG. 4.6b demonstrate that sporulation can be regulated in an inducible manner in C. sporogenes. However, only strains harbouring plasmids with spo0A driven by Pfdx4 and Pfdx4*, both located upstream of riboswitch-E, produced no spores in the absence of theophylline. Nevertheless, the spore count in the presence of the inducer was reduced by approximately 10³-fold and 10⁵-fold in Pfdx4-E and Pfdx4*-E, respectively, when compared to the WT strain. As Pfdx4-E and Pfdx4*-E are among the systems that exhibited lower levels of catP expression in the uninduced state (see FIG. 4.6a), these results indicate that a tight control of spo0A expression is required to abolish spore formation. Sporulation under the control of any other riboswitch downstream of Pfdx4 and Pfdx4* resulted in spore counts close to the detection limit. This agreed with the low expression levels of CAT observed when these promoters, combined with any riboswitch, regulate catP expression. FIG. 4.7 shows the plates onto which heat-treated cultures of the conditionally sporulating strain carrying Pfdx4*-E were plated, and how growth compares to the WT strain both, in the presence and absence of induction. Strains harbouring any other system were capable of sporulating in the absence of inducer, with the amount of spores higher in those systems exhibiting higher CAT background expression levels. Strains containing spo0A regulated by any riboswitch located downstream of Pfdx or Pfdx*, as well as in the cases of the LAC system and the RiboLac, resulted in spore counts after induction comparable to those of the WT strain. Considering that the expression of catP under the control of riboswitches located downstream of Pfdx in the presence of the inducer is, for example, 10-fold lower than that under the control of the induced LAC system, these data indicate that at a certain level of Spo0A sporulation efficiency reaches saturation. Higher expression of spo0A (i.e., Pfdx*-, ParaE*-, LAC and RiboLac) did not further increase spore counts after 120 hours.

### Chromosomally Stable Conditionally Sporulating Strains

Progression towards a stable strain capable of conditionally forming spores required the integration of spo0A, driven by an inducible system, into the chromosome of the clostridial host. Given that the level of repression and dynamic range of the different inducible systems regulating spo0A once integrated into the chromosome was unknown, it was decided to integrate several systems so that a wide range of expression profiles was covered. These included the two systems that were able to repress spore formation in the plasmid-based sporulating strains (i.e., Pfdx4-E and Pfdx4*-E); the promoters Pfdx, Pfdx* and ParaE* located upstream of the riboswitches -E and -G; the LAC system, and; the RiboLac. Although riboswitches -E and -G resulted in similar levels of plasmid-based spore counts in the presence and absence of the inducer, it was expected that expression from the single copy chromosome would result in observable differences, which could translate into a different profile of spore formation.

All inducible system-spo0A sequences were ligated into pMTL-JH29T and integrated into the genome of C. sporogenes at the pyrE locus, as previously described, and the sporulation capacity of the resultant mutants analysed. Accordingly, synchronized cultures of the designated strains were cultivated in TGY medium, in the presence and absence of induction, and total and heat-resistant CFU were enumerated after 120 hours of growth (FIG. 3.8).

Analysis of the sporulation capacity of strains containing spo0A driven by an inducible system integrated into the chromosome revealed one conditionally sporulating strain with a tight regulation of spore formation, resulting in no spores in the absence of the inducer and spore counts comparable to those of the WT strain after induction. This system was the RiboLac. Surprisingly, only the strong ParaE* promoter, out of all the integrated theophylline responsive riboswitches, was able to express spo0A, after induction, to an extent that cells were capable of activating the sporulation program; however, sporulation capacity was reduced by 10⁴-fold and 10⁶-fold relative to the WT strain, when regulated by riboswitches -E and -G, respectively. The rest of the strains with spo0A controlled by a riboswitch did not sporulate, either in the absence or in the presence of induction. Since these systems exhibited various levels of catP expression and induction profiles in previous assays (FIG. 4.6), these data support the idea that a certain threshold of Spo0A might be needed to activate the sporulation programme in C. sporogenes, and that this level is not reached when some of these systems are integrated into the chromosome. Both, the LAC system and the RiboLac restored sporulation capacity of the respective strains to WT levels after induction; however, background spo0A expression from the LAC system was enough to trigger the formation of spores, indicating the superior ability of the dual system RiboLac to tightly regulate gene expression (FIG. 4.9 and FIG. 4.8). In all cases, total CFU was higher in the presence of induction, reaching comparable counts to WT in ::LAC-spo0A and ::RiboLac-spo0A strains.

### Characterization of the Stable Conditionally Sporulating C. Sporogenes::Ribolac-spo0A Strain

The data obtained had demonstrated that the RiboLac is sufficiently tightly regulated to generate, after 120 hours, an asporogeneous strain when grown in the absence of inducers, and capable of forming spores in a WT fashion when cultivated at maximum induction (2 mM theophylline and 10 mM lactose). It was, therefore, of interest to investigate how changes in Spo0A levels, obtained via induction with various concentrations of inducers, would affect the overall sporulation efficiency (FIG. 4.10). As expected, a minimum concentration of inducers was required to activate the sporulation program. Above that level, increasing concentrations of theophylline and lactose showed a dose-dependent increase in heat-resistant CFUs, reaching the maximum levels (>10⁷ CFU/mL) from 0.5 mM theophylline and 10 mM lactose. In agreement with previous experiments, cell viability was slightly reduced in the absence of, or at very low concentrations of inducers, and increased with higher concentrations and therefore with increased expressions of spo0A.

Once the dose-dependent sporulation profile of C. sporogenes::RiboLac-spo0A was determined, it was decided to investigate if the induction of spo0A had any effect on the dynamics of the sporulation process. Accordingly, spore production was monitored over time with three different concentrations of inducers, chosen on the basis of the range of spore counts obtained in the dose-dependency assay: 0 mM theophylline and 0 mM lactose (no sporulation); 0.1 mM theophylline and 10 mM lactose (medium induction), and; 2 mM theophylline and 10 mM lactose (maximum induction) (FIG. 4.11a). In agreement with previous assays, no spores could be detected based on heat-resistant CFU counts in the absence of inducers. Maximum spore titres were observed at 120 and 72 hours, corresponding to 6.7×10⁵±3.17×10⁵ CFU/mL and 4.8×10⁷±9.5×10⁶ CFU/mL in cultures supplemented to generate medium and maximum induction, respectively. Taking into account the total CFU observed at time 0, which is similar in all conditions, these values correspond to a sporulation efficiency of 0.19% (0.1 mM theophylline and 10 mM lactose) and 13.9% (2 mM theophylline and 10 mM lactose). This indicates a delay in the activation of the sporulation process, independent of cell viability (see total CFU at 24 and 48 hours in both cases), which correlates with the level of expression of spo0A and which had been previously observed when analysing the sporulation efficiency of the complemented strain, C. sporogenes Δspo0ACOMP (FIG. 4.4). The delay in spore formation between WT and the conditionally sporulating strain was only significant during the first 48 hours of cultivation (FIG. 6.11c). After 120 hours, total CFU was comparable to the WT strain only if the ::RiboLac-spo0A strain had been exposed to maximum induction. The inducing agents neither had an effect on the final spore counts observed in WT cultures nor changed the dynamics on the sporulation process (FIG. 4.11b). The optical density of the cultures (OD600), which had been monitored over the first 24 hours of growth (FIG. 4.11d), showed a slight decrease in cell density during the first 12 hours; from this time onwards, it reached optical density values comparable to the WT strain and remained constant over the remainder of the experiment.

1. A spore-forming cell comprising a first nucleotide sequence encoding a riboswitch and a transcriptional activator,
wherein the riboswitch modulates translation of the transcriptional activator in response to a first inducer, and
a second nucleotide sequence encoding a target gene that regulates spore formation,
wherein the target gene is expressed in response to a second inducer and translation of the transcriptional activator.

2. The spore-forming cell according to claim 1, wherein the spore-forming cell is a synthetic biological cell, a bacterium, a plant, an algae, a fungi or a protozoa.

3. The spore-forming cell according to claim 2, wherein the bacterium is a species selected from the group consisting of C. acetobutylicum, C. aerotolerans, C. autoethanogenum, C. baratii, C. beijerinckii, C. bifermentans, C. botulinum, C. butyricum, C. cadaveris, C. cellulolyticum, C. cellulovorans, C. chauvoei, C. clostridioforme, C. colicanis, C. difficile (now renamed Clostridioides difficile), C. estertheticum, C. fallax, C. feseri, C. formicaceticum, C. histolyticum, C. innocuum, C. kluyveri, C. ljungdahlii, C. lavalense, C. novyi, C. oedematiens, C. paraputrificum, C. pasteurianum, C. perfringens, C. phytofermentans, C. piliforme, C. ragsdalei, C. ramosum, C. roseum, C. saccharoperbutylacetonicum, C. scatologenes, C. septicum, C. sordellii, C. sporogenes, C. sticklandii, C. tertium, C. tetani, C. thermocellum, C. thermosaccharolyticum, C. tyrobutyricum, C. paprosolvens, C. saccharobutylicum, C. carboxidovorans, C. scindens, or C. autoethanogenum.

4. The spore-forming cell according to any one of the preceding claims, wherein the riboswitch comprises or consists of an aptamer domain, which is capable of specifically binding to an inducer, and an expression platform, which undergoes a conformational change (in response to the binding of the inducer to the aptamer domain) that promotes translation of the transcriptional activator.

5. The spore-forming cell according to any one of the preceding claims, wherein the riboswitch modulates translation of a transcriptional activator in response to contact of the aptamer domain with a (first) inducer, preferably the riboswitch modulates translation of the transcriptional activator, in response to contact with an inducer, by positively regulating translation or transcription of the transcriptional activator.

6. The spore-forming cell according to claim 4 or claim 5, wherein the aptamer domain of the riboswitch specifically binds the (first) inducer.

7. The spore-forming cell according to claim 6, wherein the nucleotide sequence encoding the riboswitch is substantially as set out in SEQ ID NO. 3, 4, 5, 6, 7, 8 or 9, or a variant or fragment thereof.

8. The spore-forming cell according to any one of the preceding claims, wherein the (first) inducer is theophylline.

9. The spore-forming cell according to any one of the preceding claims, wherein the second inducer is lactose.

10. The spore-forming cell according to any one of the preceding claims, wherein the transcriptional activator is the transcription factor, BgaR.

11. The spore-forming cell according to claim 10, wherein the nucleotide sequence encoding the transcriptional activator is substantially as set out in SEQ ID NO. 10, or a variant or fragment thereof.

12. The spore-forming cell according to claim 10 or 11, wherein the second nucleotide sequence encodes a DNA-binding site of BgaR that is substantially as set out in SEQ ID NO. 11, or a variant or fragment thereof.

13. The spore-forming cell according to any one of the preceding claims, wherein the target gene is a gene that controls sporulation, preferably in bacteria.

14. The spore-forming cell according to any one of the preceding claims, wherein the spore-forming cell is only able to sporulate in the presence of the first and/or the second inducer.

15. The spore-forming cell according to any one of the preceding claims, wherein the target gene is spo0A.

16. The spore-forming cell according to any one of the preceding claims, wherein the target gene is encoded by a nucleotide sequence substantially as set out in SEQ ID NO. 12, or a variant or fragment thereof.

17. The spore-forming cell according to any one of the preceding claims, wherein the first nucleotide sequence and the second nucleotide sequence are encoded by at least one genetic construct.

18. A method of culturing a spore-forming cell, the method comprising culturing the spore-forming cell according to any one of claims 1 to 17 in the absence of a first and/or second inducer.

19. The method according to claim 18, wherein the spore-forming cell is contacted with a first inducer and a second inducer in order to induce the formation of a spore.

20. A method of creating a spore, the method comprising contacting the spore-forming cell of any one of claims 1 to 18 with a first inducer and a second inducer.

21. A spore obtained or obtainable from a method according to claim 20.

22. A spore-forming cell according to any one of claims 1 to 18 or a spore according to claim 21 for use in therapy.

23. A spore-forming cell according to any one of claims 1 to 18 or a spore according to claim 21 for use in treating or preventing treating diseases and disorders associated with the gut microbiome, such as a C. difficile infection (CDI), Irritable Bowel Disease (IBD), or cancer (bowel cancer).

24. A spore-forming cell according to any one of claims 1 to 18 or a spore according to claim 21 for use in treating or preventing a solid tumour.

25. A spore-forming cell according to any one of claims 1 to 18 for use in preventing the contamination of production facilities with spores during the manufacture from Clostridium of a biochemical, protein or other commercial product.

26. A riboswitch having positive regulatory activity.

27. The riboswitch according to claim 26, wherein a riboswitch having positive regulatory activity comprises a nucleotide sequence substantially as set out in SEQ ID NO. 3, 4, 5, 6, 7, 8 or 9, or a variant or fragment thereof.

28. A genetic construct comprising or consisting of a riboswitch according claim 26 or 27.

29. A genetic construct comprising or consisting of a nucleotide sequence encoding a riboswitch and a transcriptional activator,
wherein the riboswitch modulates translation of the transcriptional activator in response to an inducer and the transcriptional activator promotes transcription of a target gene.

30. At least one genetic construct comprising or consisting of:
a first nucleotide sequence encoding a riboswitch and a transcriptional activator,
wherein the riboswitch modulates translation of the transcriptional activator in response to a first inducer, and
a second nucleotide sequence encoding a target gene,
wherein the target gene is expressed in response to a second inducer and translation of the transcriptional activator.

31. A genetic construct according to claim 29 or 30, wherein the genetic construct is capable of reducing ‘leakiness’ (i.e. basal transcription and/or translation of a target gene) by at least 10%, 20%, 30%, 40%, 50%, 60%, 70%, 80% or 90%.

32. A vector comprising a genetic construct according to claim 29, 30 or 31.

33. A host cell comprising a genetic construct according to claim 29, 30 or 31 or a vector according to claim 32.

34. A method of inducing translation, in a cell, of an RNA molecule encoding a (first) nucleotide sequence, the method comprising:
transforming a host cell with a genetic construct according to claim 29, 30 or 31 or a vector according to claim 32 to create a transformed host cell; and
contacting the transformed host cell with a (first) inducer to induce translation of the RNA of the (first) nucleotide sequence.

35. A method of inducing expression, in a cell, of a target gene, the method comprising:
transforming a host cell with a genetic construct according to claim 29, 30 or 31 or a vector according to claim 32 to create a transformed host cell; and
contacting the transformed host cell with a first inducer and a second inducer to induce expression of the target gene in the transformed host cell.
